(19) United States
(12) Patent Application Publication
(10) Pub. No.: US 2026/0102347 A1
(43) Pub. Date: Apr. 16, 2026

(54) Sulfur-Containing Ionizable Lipids for the Delivery of Therapeutic Agents

(71) Applicant: NanoVation Therapeutics Inc., Vancouver (CA)

(72) Inventor: Deaglan Arnold, London (GB)

(73) Assignee: NanoVation Therapeutics Inc., Vancouver, BC (CA)

(21) Appl. No.: 19/115,823

(22) PCT Filed: Sep. 27, 2023
(86) PCT No.: PCT/CA2023/051273

§ 371 Date: Mar. 27, 2025

Related U.S. Application Data
(60) Provisional application No. 63/410,261, filed on Sep. 27, 2022.

(51) Int. Cl. A61K 9/1272; A61K 9/51; A61K 31/7105; C07C 323/12; C07C 323/17; C07C 323/25; C07D 317/28; C07D 319/06; C07D 405/06

(52) U.S. Cl. CPC A61K 9/1272; A61K 9/5123; A61K 31/7105; C07C 323/12; C07C 323/17; C07C 323/25; C07D 317/28; C07D 319/06; C07D 405/06; C07C 2601/14

(57) ABSTRACT

Provided are novel sulfur-containing lipids and nanoparticles containing such lipids and a cargo molecule, such as a nucleic acid, methods to formulate said lipids with nucleic acids to produce lipid nanoparticles and chemical routes for making said lipids. The lipids may have the structure of Formula A as defined herein. Formula A

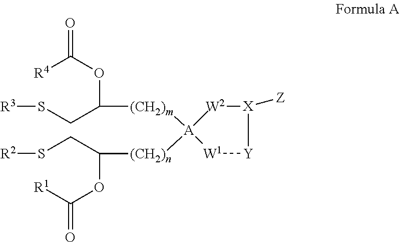

### TECHNICAL FIELD

Provided herein are sulfur-containing lipids that may be formulated in a delivery vehicle so as to facilitate the encapsulation of cargo, such as, without limitation, nucleic acids (e.g., RNA or DNA), proteins, peptides, pharmaceutical drugs and salts thereof.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of a lipid nanoparticle (LNP) is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the LNPs are taken up by a cell by endocytosis, the ionizability of these lipids at low pH enables endosomal escape. This in turn enables the nucleic acid to be released into the intracellular compartment.

An earlier example of an LNP product approved for clinical use and reliant on ionizable lipid is Onpattro®. Onpattro® is a lipid nanoparticle-based short interfering RNA (siRNA) drug for the treatment of polyneuropathies induced by hereditary transthyretin amyloidosis. Onpattro® is reliant on an ionizable lipid referred to as “DLin-MC3-DMA” or more commonly “MC3”, 1 (FIG. 1), by investigators. Furthermore, MC3 represents an evolution of a structurally related ionizable lipid, referred to by investigators as “KC2”, 2 (FIG. 1). MC3 is considered a state-of-the art ionizable lipid for the delivery of siRNA, requiring about 3 times less siRNA than KC2, although KC2 is superior in other applications, and it remains a valuable research tool.

While the foregoing ionizable lipids are especially efficacious for the delivery of siRNA-containing LNPs to hepatic cells, they are much less effective for the hepatic delivery of mRNA-containing LNPs. To illustrate, mRNA vaccines, including the COVID-19 Pfizer/BioNTech and Moderna vaccines, rely on lipid nanoparticles to deliver mRNA to the cytoplasm of liver cells. After entry into the host cell, the mRNA is transcribed to produce antigenic proteins. In the case of the COVID-19 vaccines, the mRNA encodes the highly immunogenic Sars-Cov-2 spike protein. Such vaccines, however, incorporate other types of ionizable lipids besides MC3 or KC2. In particular, the Pfizer/BioNTech vaccine comprises an ionizable lipid referred to as “ALC-0315”, 3 (Scheme 1), and the Moderna vaccine comprises an ionizable lipid referred to as “SM-102”, 4.

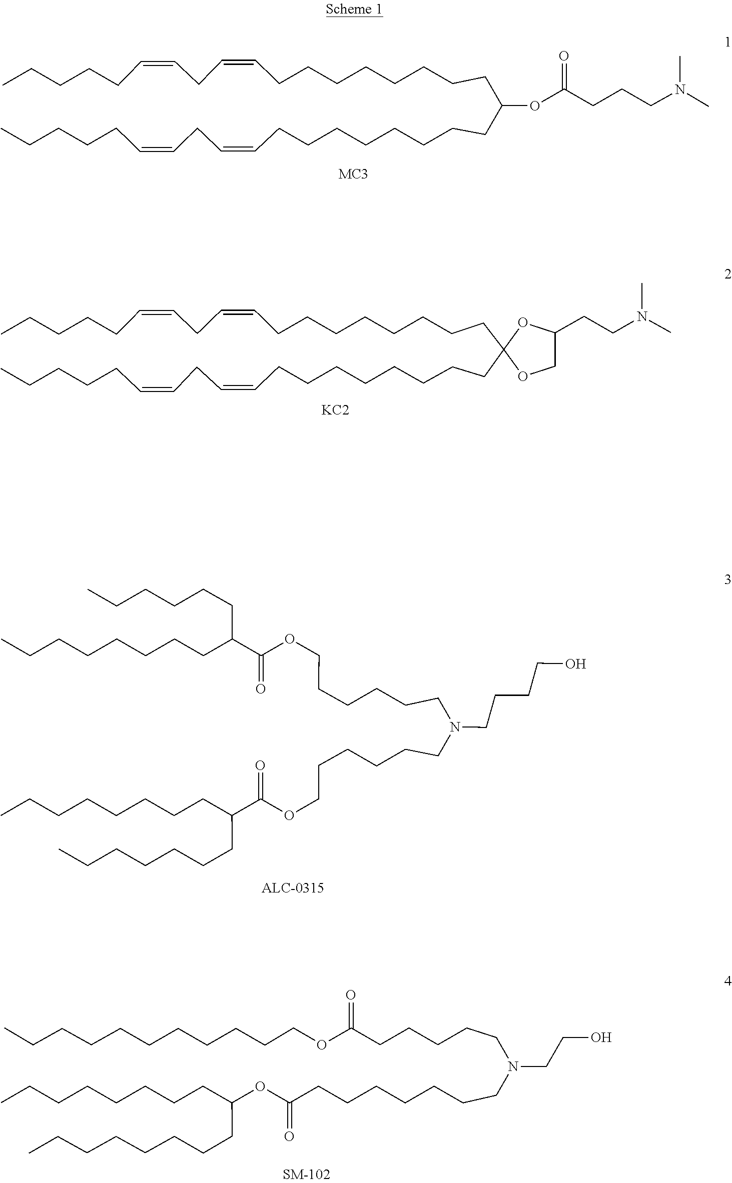

Furthermore, the above lipids were optimized for delivery of therapeutic nucleic acids to the liver. However, there remains a need to develop new lipids for the delivery of charged cargo, such as nucleic acids to other organs, such as the spleen, lungs, bone marrow, skin, etc. The delivery of therapeutics beyond the liver would expand the clinical utility of LNPs to target disease conditions that affect tissues and organs beyond the liver. There is also an ongoing need to develop LNPs with improved delivery of nucleic acid or other charged cargo to the liver.

The present disclosure seeks to address one or more of the above identified problems and/or provides useful alternatives to known products and/or compositions for the delivery of nucleic acid or other charged cargo.

### Definitions

As used herein, “type 1 ionizable head” or “MC-type ionizable head” refers to a moiety that has a head group of the lipid of Formula I below, or equivalents thereof, with n ranging from 1 to 5:

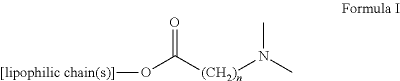

As used herein, “type 2 ionizable head” “KC-type ionizable head” refers to a moiety that has a head group of the lipid of Formula II below, or equivalents thereof, with n ranging from 1 to 5:

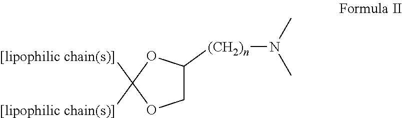

As used herein, “type 3 ionizable head” refers to a moiety that is the head group of the structure as defined by Formula III below, or equivalents thereof, with m and n independently ranging from 1 to 5:

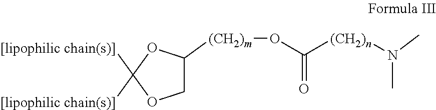

As used herein, “type 4 ionizable head” refers to a moiety that is the head group of the structure as defined by Formula IV below, or equivalents thereof, with R=C₁-C₆ alkyl or cycloalkyl, and with m and n independently ranging from 2 to 5:

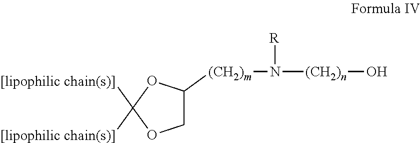

As used herein, “type 5 ionizable head” refers to a moiety that is the head group of the structure as defined by Formula V below, or equivalents thereof, with m and n independently ranging from 1 to 5:

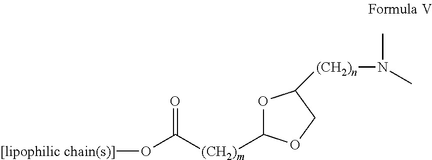

As used herein, “type 6 ionizable head” refers to a moiety that is the head group of the structure as defined by Formula VI below, or equivalents thereof, with R=C₁-C₆ alkyl or cycloalkyl and with m ranging from 1 to 5, and n, independently, ranging from 2 to 5:

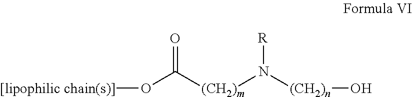

As used herein, “type 7 ionizable head” refers to a moiety that is the head group of the structure as defined by Formula VII below, or equivalents thereof, with R=C₁-C₆ alkyl or cycloalkyl, and with n ranging from 1 to 5:

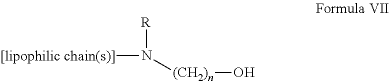

As used herein, “type 8 ionizable head” refers to a moiety that is the head group of the structure as defined by Formula VIII below, or equivalents thereof, with R=C₁-C₆ alkyl or cycloalkyl, and with n ranging from 1 to 5:

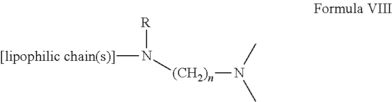

As used herein, “type 9 ionizable head” refers to a moiety that is the head group of the structure as defined by Formula IX below, or equivalents thereof, with m and n independently ranging from 1 to 5:

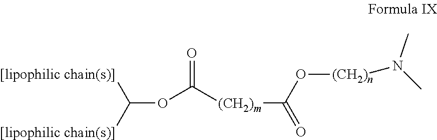

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a cLog P) that is greater than 8.

As used herein, the term “lipophilic chain” refers to an alkyl group bonded to a nitrogen or carbon atom of the lipid, said alkyl group comprising at least 6 C atoms and optionally comprising C═C double bonds, and/or ring structures, and/or carbonyl groups, and/or heteroatoms such as N, O, S, and such that the parent compound of said alkyl group has a CLog P of at least 6.

For example, lipid MC3, 1, and lipid KC2, 2, have a pair of lipophilic chains derived from (6Z,9Z)-octadeca-6,9-diene, which has a CLog P of 9.25:

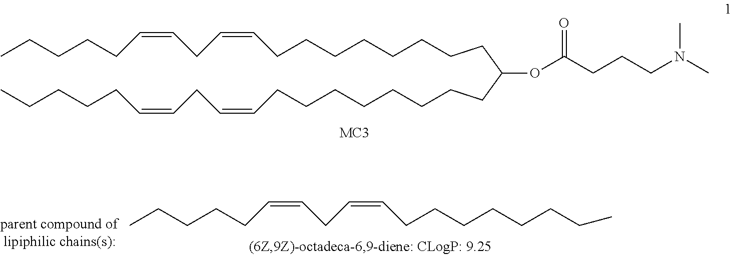

Lipid ALC-0315, 3, has a pair of lipophilic chains derived from hexyl 2-hexyldecanoate, which has a CLog P of 10.01:

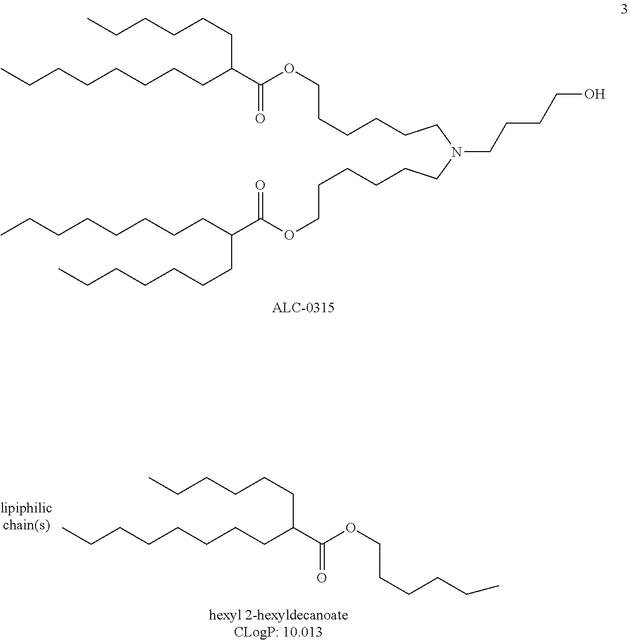 [SMILES: CCCCCCCCC(CCCCCC)C(=O)OCCCCCCN(CCCCO)CCCCCCOC(=O)C(CCCCCCC)CCCCCCCC ; CCCCCCCCC(CCCCCC)C(=O)OCCCCCC]

Lipid SM-102, 4, has one lipophilic chain derived from undecyl hexanoate, which has a CLog P of 7.59, and one lipophilic chain derived from heptadecane-9-yl octanoate, which has a CLog P of 11.6:

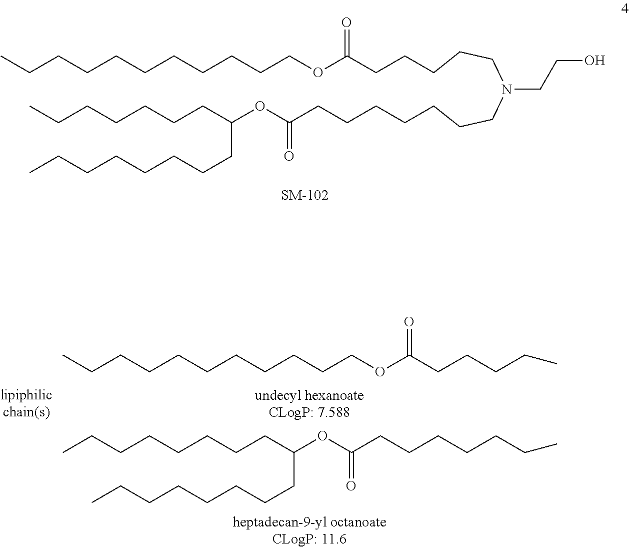

As used herein, the term “alkyl” or “alkyl group” is a carbon-containing chain that is linear or branched and that optionally comprises C═C double bonds and/or ring structures, and that is optionally substituted.

As used herein, the term “Cₘ to Cₙ alkyl” or “Cₘ to Cₙ alkyl group” refers to a linear or branched carbon chain having a total minimum of m carbon atoms and up to n carbon atoms, and that is optionally unsaturated and optionally substituted. For example, a “C₁ to C₃ alkyl” or “C₁ to C₃ alkyl group” is an alkyl having between 1 and 3 carbon atoms.

The term “optionally substituted” with reference to an alkyl means that at least one hydrogen atom of the alkyl group can be replaced by a non-hydrogen atom or group of atoms (i.e., a “substituent”), and/or the alkyl is interrupted by one or more substituents comprising heteroatoms selected from O, S and NR′, wherein R′ is as defined below. Non-limiting examples of groups that may replace a hydrogen atom include halogen; alkyl groups; cycloalkyl groups; oxo groups (═O); hydroxyl groups (—OH); —(C═O) OR′; —O(C═O)R′; —C(═O) R′; —OR′; —S(O)ₓR′; —S—SR′; —C(═O) SR′; —SC(═O)R′; —NR′R′; —NR′C(═O)R′; —C(═O)NR′R′; —NR′C(═O)NR′R′; —OC(═O)NR′R′; —NR′C(═O)OR′; —NR′S(O)ₓNR′R′; —NR′S(O)ₓR′; and —S(O)ₓNR′R′, wherein R′ at each occurrence is independently selected from H, C₁-C₁₅ alkyl or cycloalkyl, and x is 0, 1 or 2.

As used herein, the term “helper lipid” means a compound selected from: a sterol such as cholesterol or a derivative thereof; a diacylglycerol or a derivative thereof, such as a glycerophospholipid, including phosphatidic acid (phosphatidate) (PA), phosphatidylethanolamine (cephalin) (PE), phosphatidylcholine (PC), phosphatidylserine (PS), and the like; and a sphingolipid, such as a ceramide, a sphingomyelin, a cerebroside, a ganglioside, or reduced analogues thereof, that lack a double bond in the sphingosine unit. An example of a diacylglycerol derivative is a glycerophospholipid-cholesterol conjugate in which one of the acyl chains is substituted with a moiety comprising cholesterol. The term encompasses lipids that are either naturally-occurring or synthetic.

As used herein, the term “delivery vehicle” includes any preparation in which the lipid described herein is capable of being formulated and includes but is not limited to delivery vehicles comprising helper lipids.

As used herein, the term “nanoparticle” is any suitable particle in which the lipid can be formulated and that may comprise one or more helper lipid components. The one or more lipid components may include an ionizable lipid prepared by the method described herein and/or may include additional lipid components, such as the one or more helper lipid components. The term includes, but is not limited to, vesicles with one or more bilayers, including multilamellar vesicles, unilamellar vesicles and vesicles with an electron-dense core. The term also includes polymer-lipid hybrids, including particles in which the lipid is attached to a polymer.

As used herein, the term “encapsulated,” with reference to incorporating a cargo molecule (e.g., mRNA) within a delivery vehicle refers to any association of the cargo with any component or compartment of the delivery vehicle such as a nanoparticle.

The term “pharmaceutically acceptable salt” with reference to a form of the lipid of the disclosure in a protonated form (i.e., charged) and/or as part of a pharmaceutical formulation in which an LNP is formulated refers to a salt prepared from pharmaceutically acceptable, non-toxic acids, including inorganic and organic acids.

The article “a” or “an” as used herein is meant to include both singular and plural, unless otherwise indicated.

### SUMMARY

The present disclosure is based, at least in part, on the surprising discovery that LNP formulations of nucleic acids comprising ionizable lipids that incorporate at least one lipophilic chain substituted with a sulfur atom and an ester moiety are more potent than then benchmark MC3 for liver delivery of therapeutic RNA. As further described herein, such lipids may exhibit a different organ selectivity relative to known lipids. In particular, non-limiting examples described herein demonstrate that such lipids promote the delivery of mRNA selectively to the spleen more efficiently than other known lipids. In addition, the chemical synthesis of the lipids of certain embodiments herein is more straightforward and/or economical than that of known lipids.

Other objects, features, and advantages of the present disclosure will be apparent to those of skill in the art from the following detailed description and figures.

According to one aspect of the disclosure, there is provided a lipid having a structure of Formula A:

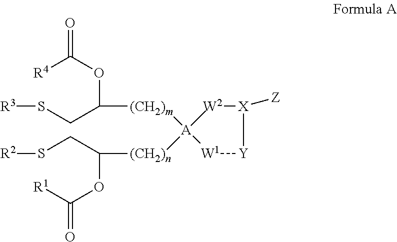

or a pharmaceutically acceptable salt thereof;
wherein
   m is 4 to 8; and
   n is 4 to 8;
   R¹, R², R³, and R⁴ are linear or branched optionally substituted C₃ to C₂₀ alkyl and optionally comprising 0-2 carbon-carbon double bonds;
   A is either C or N, and
      if A is C, then W¹ and Y are either bonded to each other or not bonded to each other, and
         if W¹ and Y are bonded to each other, then
         W¹ is O or S;
         W² is O or S;
         X is CH;
         Y is (CH₂)_{q}, wherein q is 1 or 2;
         Z is selected from one of structures a-c below, wherein the wavy line represents the bond to X
         a.

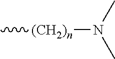

type 2 ionizable head group;
b.

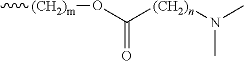

type 3 ionizable head group;
c.

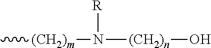

type 4 ionizable head group;
if W¹ and Y are not bonded to each other, then
W¹ is H;
W² is O, S, NH or NR^{2a}, wherein R^{2a} is a C₁ to C₄ alkyl optionally substituted with an OH group; and the moiety

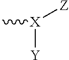

of Formula A is a group selected from structures d-h below, wherein the wavy line represents the bond to W²
d.

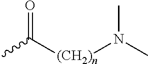

type 1 ionizable head group;
e.

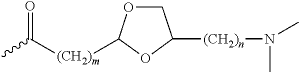

type 5 ionizable head group;
f.

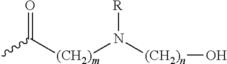

type 6 ionizable head group;
g. [symbol] (CH₂)ₙ—OH type 7 ionizable head group;
h.

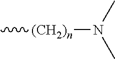

type 8 ionizable head group;
i.

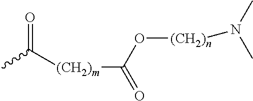

type 9 ionizable head group;
if A is N, then
W¹ and Y are absent;
W² and X together form a group of structure (CR^{a}R^{b})ₚ, wherein R^{a} and R^{b} are independently H or C₁-C₈alkyl or cycloalkyl, and wherein p is 2 to 6; and
Z is OH or NR′R″, wherein R′ and R″ are independently optionally substituted C₁-C₈alkyl or cycloalkyl, or wherein R′ and R″ together with the N atom of NR′R″, form an optionally substituted heterocyclic ring that incorporates the N atom to which the R′ and R″ are each bound.

According to an embodiment of the foregoing aspect, at least one of R¹ and R⁴ may be, independently, a moiety of Formula B, wherein:

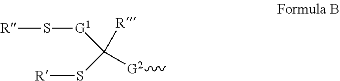

R′ and R″ are, independently, linear or branched optionally substituted C₃ to C₁₂ alkyl groups and optionally comprising 0-2 carbon-carbon double bonds;
R′″ is H or a linear, branched, or cyclic optionally substituted C₁ to C₆ alkyl group; and
G¹ and G² are, independently, (CR^{a}R^{b})ₚ, wherein R^{a} and R^{b} are each independently selected from H or optionally substituted C₁-C₈alkyl or cycloalkyl, wherein p is 0 to 6.

According to the foregoing aspect or embodiments thereof, A³ may be N, W¹ and Y are absent, W² and X together form a group of the structure (CR^{a}R^{b})ᵣ, and Z is NR′R″ and wherein the heterocyclic group that incorporates the N atom to which R′ and R″ are bound is pyrrolidine, piperidine or morpholine.

According to the foregoing aspect or embodiments thereof, A³ may be a carbon atom.

According to the foregoing aspect or embodiments thereof, W¹ and Y are not bonded to each other.

According to the foregoing aspect or embodiments thereof, W² may be O.

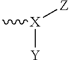

According to the above aspect or embodiments thereof, the lipid has a structure of any one of the compounds 5-21 or pharmaceutically acceptable salts thereof as set forth in Table 1 herein.

According to a further aspect of the disclosure, there is provided a lipid or a pharmaceutically acceptable salt thereof comprising:
a protonatable amino head group;
two lipophilic chains, wherein the amino head group has a central nitrogen atom or carbon atom to which each of the two lipophilic chains are directly bonded;
at least one of the lipophilic chains has the formula:

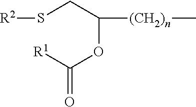

wherein R¹ and R² are, independently, linear or branched optionally substituted C₃-C₂₀ alkyl and optionally with varying degrees of unsaturation;
n is 4 to 8;
each lipophilic chain has between 15 and 40 carbon atoms in total; and
wherein the lipid has (i) a pKₐ of between 6 and 8; and (ii) a log P of at least 11.

According to a further embodiment of any of the foregoing aspects of the disclosure, the lipid, when formulated in a lipid nanoparticle comprising an mRNA, results in an increase in biodistribution of the lipid nanoparticle of at least about 10% in the liver and/or one or more extrahepatic tissues, such as the spleen, relative to a lipid nanoparticle containing DLin-MC3-DMA as measured by luminescence of the mRNA in vivo in the liver and/or the one or more extrahepatic tissues. The assay and the formulations used to determine the biodistribution are as described in Example 2.

In another aspect, there is provided a lipid nanoparticle comprising the lipid of any one of the foregoing aspects or embodiments thereof and a nucleic acid.

The lipid nanoparticle may comprise a helper lipid and a hydrophilic polymer-lipid conjugate. The helper lipid may be selected from cholesterol, a diacylglycerol and a sphingolipid.

According to another aspect, there is provided a lipid nanoparticle comprising:
an ionizable lipid with two lipophilic chains directly bonded to a central nitrogen or carbon atom in which at least one of the lipophilic chains has the formula:

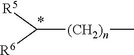

n is 4 to 8;
wherein the * represent a carbon branch point;
wherein R⁵ and R⁶ are each independently linear or branched substituted C₃-C₃₀ alkyl groups;
wherein one of R⁵ and R⁶ is substituted with an ester group and the other of R⁵ and R⁶ is substituted with a sulfur atom at an alpha, beta or gamma position relative to the carbon branch point;
one or more helper lipids;
a hydrophilic polymer-lipid conjugate; and
a nucleic acid.

In a further aspect, there is provided a method for administering a nucleic acid to a subject in need thereof, the method comprising preparing or providing the lipid nanoparticle as described in any of the foregoing aspects or embodiments comprising the nucleic acid and administering the lipid nanoparticle to the subject.

According to another aspect of the disclosure, there is provided a method for delivering a cargo molecule to a cell, the method comprising contacting the lipid nanoparticle as described in any aspect or embodiment as described above with the cell in vivo or in vitro. In one embodiment, the cargo molecule is a nucleic acid.

According to a further aspect of the disclosure, there is provided a use of the lipid or a pharmaceutically acceptable salt thereof as defined above or the lipid nanoparticle as defined in any aspect or embodiment described above, in the manufacture of a medicament to treat or prevent a disease, disorder or condition that is treatable and/or preventable by a nucleic acid.

According to a further aspect of the disclosure, there is provided a use of the lipid or the pharmaceutically acceptable salt thereof as defined above or the lipid nanoparticle of any one of the aspects or embodiments described above to deliver a nucleic acid to a patient to treat or prevent a disease, disorder or condition that is treatable or preventable by the nucleic acid. In one embodiment, the nucleic acid is an mRNA.

### BRIEF DESCRIPTION OF THE DRAWINGS

FIG. 1 is a bar graph showing entrapment (%), particle size and polydispersity index (PDI) of mRNA-containing lipid nanoparticles (LNPs) comprising the ionizable lipids 1 and 5-32 (Table 1). The LNPs are composed of 50/10/38.5/1.5 mol % of ionizable lipid/DSPC/chol/PEG-DMG and the amine-to-phosphate ratio (N/P) was 6.

FIG. 2A shows luminescence intensity/mg in the liver for the mRNA-containing LNPs comprising the ionizable lipids 29, 28, 18, 19, 15, 17, 14, 20, 13, 21, 1, 7, 9, 8, 11, 10, 6, 25, 23, 30, 12, 24, 16, 32, 26, 22, 31, 27 and 5 after 4 hours post-intravenous administration to CD-1 mice. The LNPs contain 50/10/38.5/1.5 mol % of ionizable lipid/DSPC/chol/PEG-DMG (N/P=6).

FIG. 2B shows luminescence intensity/mg in the spleen for the mRNA-containing LNPs comprising the ionizable lipids 19, 29, 28, 18, 10, 11, 1, 20, 21, 7, 17, 23, 12, 26, 27, 15, 8, 14, 13, 31, 24, 32, 25, 5, 16, 22, 6, 30 and 9 after 4 hours post-intravenous administration to CD-1 mice. The LNPs contain 50/10/38.5/1.5 mol % of ionizable lipid/DSPC/chol/PEG-DMG (N/P=6).

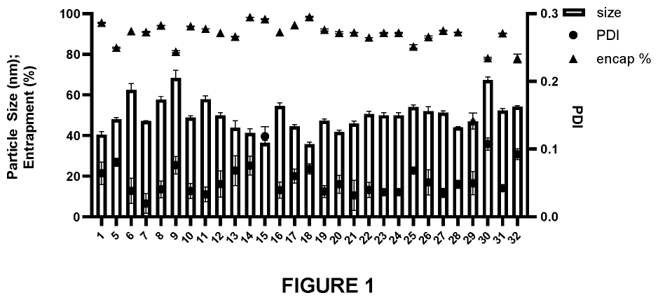

### DETAILED DESCRIPTION

Various aspects and embodiments of the disclosure are directed to ionizable lipids having structures of Formula A and pharmaceutically acceptable salts thereof.

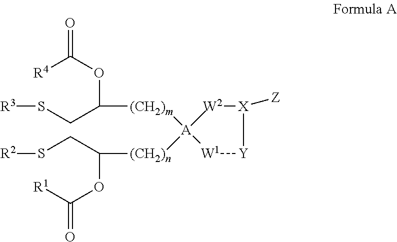

Formulations comprising such lipids find use in the delivery of nucleic acid to any target site of interest. In some embodiments, such lipids have been found to be particularly efficacious for the delivery of mRNA when formulated in a suitable delivery vehicle. In further embodiments, such lipids can be easily synthesized and prepared by processes having improved economics relative to known methods for making ionizable lipids.

### Methods to Produce Lipids of Formula A

Lipids of Formula A or pharmaceutically acceptable salts thereof can be prepared using any suitable method known to those of skill in the art. Particularly suitable methods are described below, which are exemplified, without intending to be limiting, with the synthesis of compound 5-32 of Table 1 below. Those skilled in the art would appreciate that alternative starting materials could be employed in the same sequence, leading to congeners of compound 5-32 as defined by Formula A. Therefore, the synthetic schemes set forth below are merely illustrative of select embodiments.

Certain steps of the synthesis of compounds such as 5 are described in detail in co-pending and co-owned WO 2023/173203, which is incorporated herein by reference. As described in the foregoing disclosure, one such step entails reacting an appropriate aminoalcohol or an O-protected variant thereof, represented in Scheme 2 with the generic formula 33, with a suitable alkyl halide or sulfonate, represented in Scheme 2 with the generic formula 34,

TABLE 1
structure

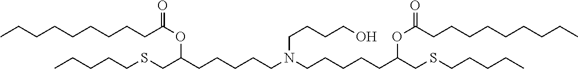 [SMILES: CCCCCCCCCC(=O)OC(CCCCCN(CCCCO)CCCCCC(CSCCCCC)OC(=O)CCCCCCCCC)CSCCCCC]

5

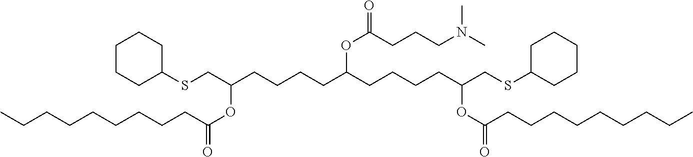 [SMILES: CCCCCCCCCC(=O)OC(CCCCC(CCCCC(CSC1CCCCC1)OC(=O)CCCCCCCCC)OC(=O)CCCN(C)C)CSC1CCCCC1]

6

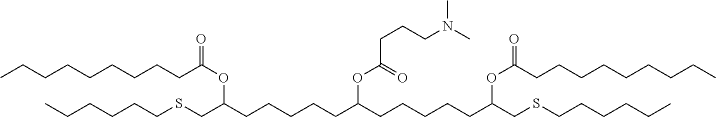 [SMILES: CCCCCCCCCC(=O)OC(CCCCCC(CCCCCC(CSCCCCCC)OC(=O)CCCCCCCCC)OC(=O)CCCN(C)C)CSCCCCCC]

7

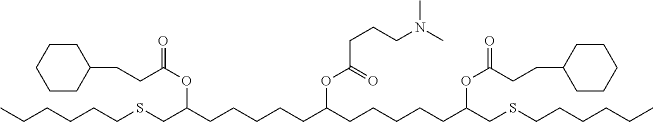 [SMILES: CCCCCCSCC(CCCCCC(CCCCCC(CSCCCCCC)OC(=O)CCC1CCCCC1)OC(=O)CCCN(C)C)OC(=O)CCC1CCCCC1]

8

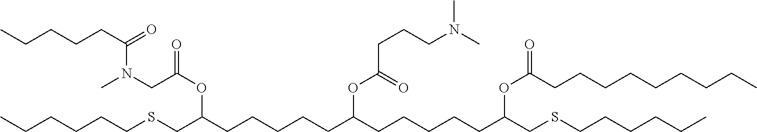 [SMILES: CCCCCCCCCC(=O)OC(CCCCCC(CCCCCC(CSCCCCCC)OC(=O)CN(C)C(=O)CCCCC)OC(=O)CCCN(C)C)CSCCCCCC]

9

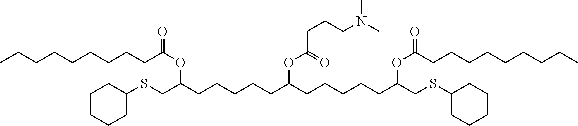 [SMILES: CCCCCCCCCC(=O)OC(CCCCCC(CCCCCC(CSC1CCCCC1)OC(=O)CCCCCCCCC)OC(=O)CCCN(C)C)CSC1CCCCC1]

10

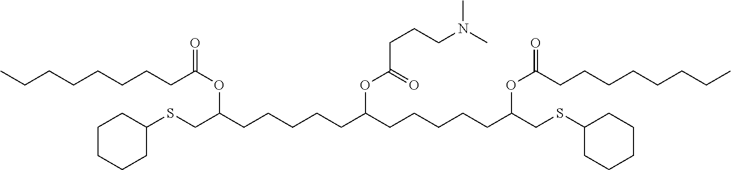 [SMILES: CCCCCCCCC(=O)OC(CCCCCC(CCCCCC(CSC1CCCCC1)OC(=O)CCCCCCCC)OC(=O)CCCN(C)C)CSC1CCCCC1]

11

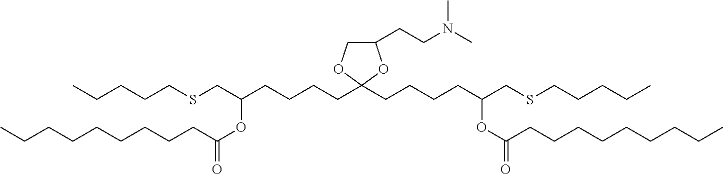 [SMILES: CCCCCCCCCC(=O)OC(CCCCC1(CCCCC(CSCCCCC)OC(=O)CCCCCCCCC)OCC(CCN(C)C)O1)CSCCCCC]

12

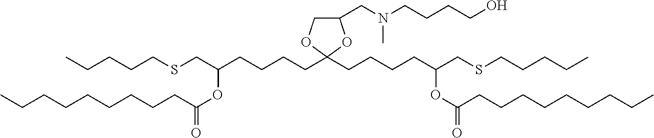 [SMILES: CCCCCCCCCC(=O)OC(CCCCC1(CCCCC(CSCCCCC)OC(=O)CCCCCCCCC)OCC(CN(C)CCCCO)O1)CSCCCCC]

13

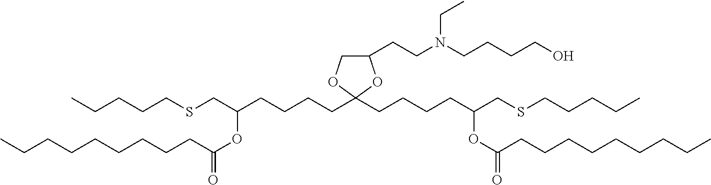 [SMILES: CCCCCCCCCC(=O)OC(CCCCC1(CCCCC(CSCCCCC)OC(=O)CCCCCCCCC)OCC(CCN(CC)CCCCO)O1)CSCCCCC]

14

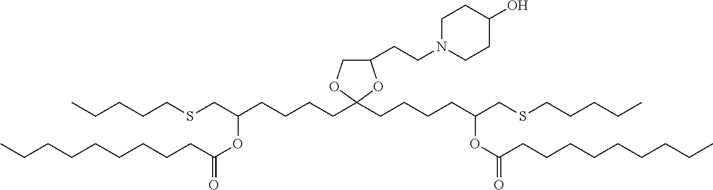 [SMILES: CCCCCCCCCC(=O)OC(CCCCC1(CCCCC(CSCCCCC)OC(=O)CCCCCCCCC)OCC(CCN2CCC(O)CC2)O1)CSCCCCC]

15

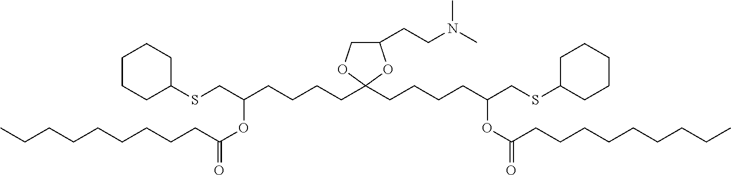 [SMILES: CCCCCCCCCC(=O)OC(CCCCC1(CCCCC(CSC2CCCCC2)OC(=O)CCCCCCCCC)OCC(CCN(C)C)O1)CSC1CCCCC1]

16

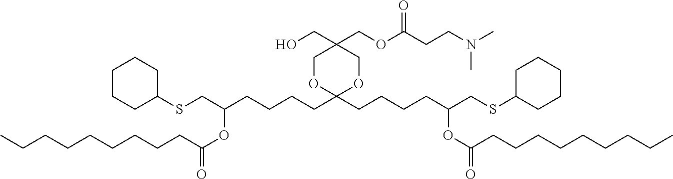 [SMILES: CCCCCCCCCC(=O)OC(CCCCC1(CCCCC(CSC2CCCCC2)OC(=O)CCCCCCCCC)OCC(CO)(COC(=O)CCN(C)C)CO1)CSC1CCCCC1]

17

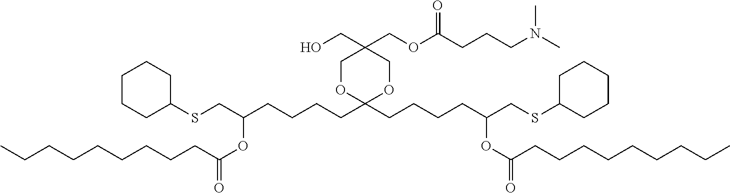 [SMILES: CCCCCCCCCC(=O)OC(CCCCC1(CCCCC(CSC2CCCCC2)OC(=O)CCCCCCCCC)OCC(CO)(COC(=O)CCCN(C)C)CO1)CSC1CCCCC1]

18

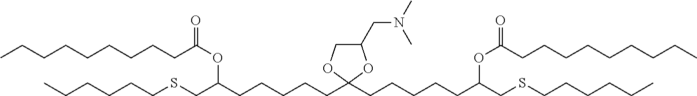 [SMILES: CCCCCCCCCC(=O)OC(CCCCCC1(CCCCCC(CSCCCCCC)OC(=O)CCCCCCCCC)OCC(CN(C)C)O1)CSCCCCCC]

19

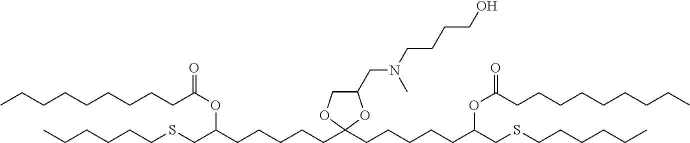 [SMILES: CCCCCCCCCC(=O)OC(CCCCCC1(CCCCCC(CSCCCCCC)OC(=O)CCCCCCCCC)OCC(CN(C)CCCCO)O1)CSCCCCCC]

20

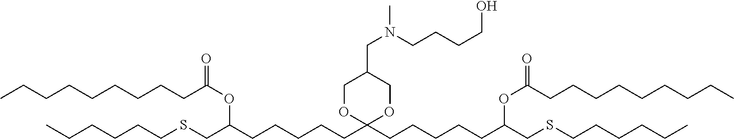 [SMILES: CCCCCCCCCC(=O)OC(CCCCCC1(CCCCCC(CSCCCCCC)OC(=O)CCCCCCCCC)OCC(CN(C)CCCCO)CO1)CSCCCCCC]

21

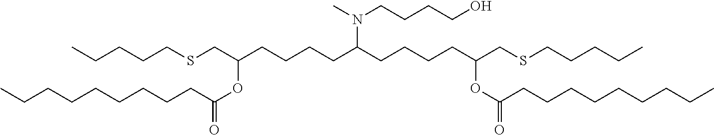 [SMILES: CCCCCCCCCC(=O)OC(CCCCC(CCCCC(CSCCCCC)OC(=O)CCCCCCCCC)N(C)CCCCO)CSCCCCC]

22

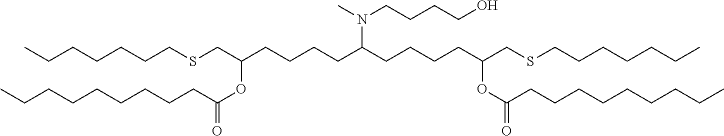 [SMILES: CCCCCCCCCC(=O)OC(CCCCC(CCCCC(CSCCCCCCC)OC(=O)CCCCCCCCC)N(C)CCCCO)CSCCCCCCC]

23

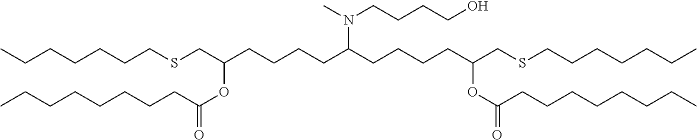 [SMILES: CCCCCCCCC(=O)OC(CCCCC(CCCCC(CSCCCCCCC)OC(=O)CCCCCCCC)N(C)CCCCO)CSCCCCCCC]

24

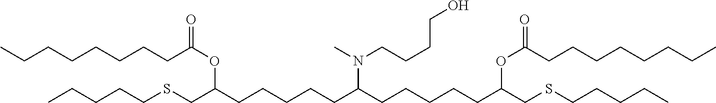 [SMILES: CCCCCCCCC(=O)OC(CCCCCC(CCCCCC(CSCCCCC)OC(=O)CCCCCCCC)N(C)CCCCO)CSCCCCC]

25

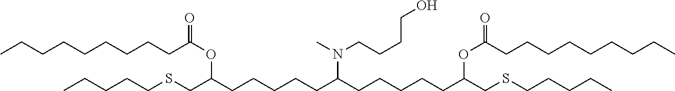 [SMILES: CCCCCCCCCC(=O)OC(CCCCCC(CCCCCC(CSCCCCC)OC(=O)CCCCCCCCC)N(C)CCCCO)CSCCCCC]

26

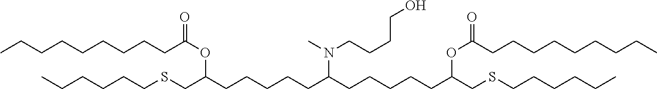 [SMILES: CCCCCCCCCC(=O)OC(CCCCCC(CCCCCC(CSCCCCCC)OC(=O)CCCCCCCCC)N(C)CCCCO)CSCCCCCC]

27

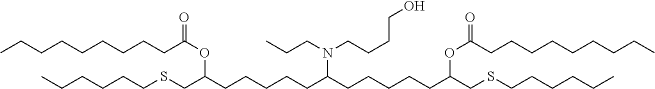 [SMILES: CCCCCCCCCC(=O)OC(CCCCCC(CCCCCC(CSCCCCCC)OC(=O)CCCCCCCCC)N(CCC)CCCCO)CSCCCCCC]

28

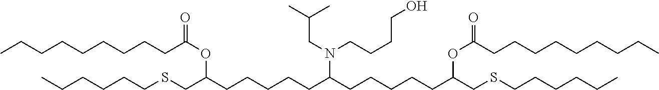 [SMILES: CCCCCCCCCC(=O)OC(CCCCCC(CCCCCC(CSCCCCCC)OC(=O)CCCCCCCCC)N(CCCCO)CC(C)C)CSCCCCCC]

29

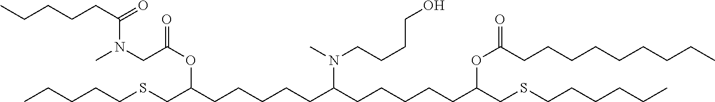 [SMILES: CCCCCCCCCC(=O)OC(CCCCCC(CCCCCC(CSCCCCC)OC(=O)CN(C)C(=O)CCCCC)N(C)CCCCO)CSCCCCCC]

30

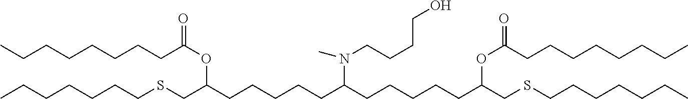 [SMILES: CCCCCCCCC(=O)OC(CCCCCC(CCCCCC(CSCCCCCCC)OC(=O)CCCCCCCC)N(C)CCCCO)CSCCCCCCC]

31

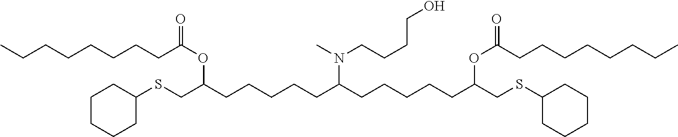 [SMILES: CCCCCCCCC(=O)OC(CCCCCC(CCCCCC(CSC1CCCCC1)OC(=O)CCCCCCCC)N(C)CCCCO)CSC1CCCCC1]

32

resulting in formation of different products depending on the conditions. Specifically, a primary amine can be converted into a corresponding secondary amine by allowing the reaction to take place in DMF at room temperature in the presence of K₂CO₃, whereupon selective mono-N-alkylation of the starting amine occurs, resulting in formation of a product represented with the generic formula 35 in Scheme 2. A secondary amine such as 35 can then be N-alkylated a second time by allowing it to react with another alkyl halide or sulfonate, represented with the generic formula 36 in Scheme 2, by heating in acetonitrile in the presence of K₂CO₃ or Na₂CO₃. Thus, secondary amine 35 is transformed into a tertiary amine of general structure 37.

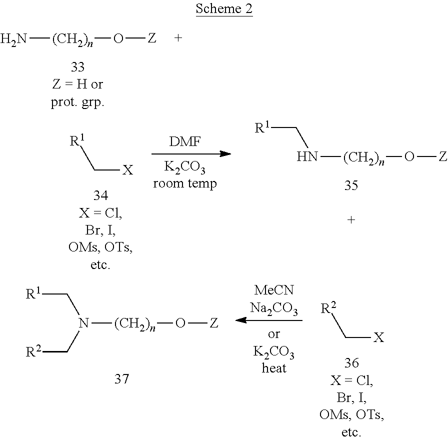

Alternatively, primary amine 33 can be doubly alkylated in a single step by heating with an alkyl halide or sulfonate 34 in acetonitrile in the presence of K₂CO₃ or Na₂CO₃, whereupon a double N-alkylation of the starting amine occurs, resulting in conversion of 33 into a tertiary amine of generic structure 38 (Scheme 3). In cases where Z in 33 or 38 is a protecting group, a deprotection step can be used to convert compound 38 into 39.

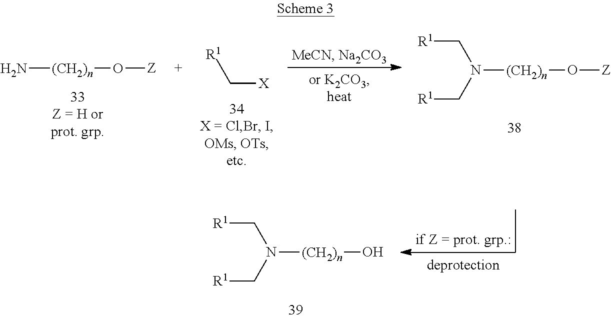

The technique outlined in Scheme 3 can be employed for the synthesis of lipid 5, in which case the primary amine is an O-protected derivative of a 4-amino-1-butanol, such as the O-TBDPS derivative 40, the alkyl halide is 41, and the product is 42 (Scheme 4), which can be transformed into lipid 5 as described below.

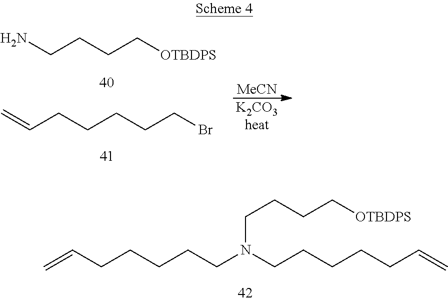

The synthesis of representative lipid 5 continues with the bis-epoxidation of the double bonds in 42 with any suitable epoxidizing reagent. For example, reaction of 42 with a peroxycarboxylic acid, such as, but not limited to, magnesium monoperoxyphthalate, peracetic acid, meta-chloroperoxybenzoic acid (MCPBA), and the like, produces 43 as a mixture of epoxide diastereomers, which are not separated. The reaction of compound 43 with a thiol such as 1-pentanethiol under basic conditions results in selective nucleophilic opening of the epoxide at the CH₂ position, leading to the formation of a bis-beta-hydroxysulfide 44, which retains the N-oxide functional group. The N-oxide is then reduced to a corresponding tertiary amine 45 by reaction with triphenylphosphine (Scheme 5).

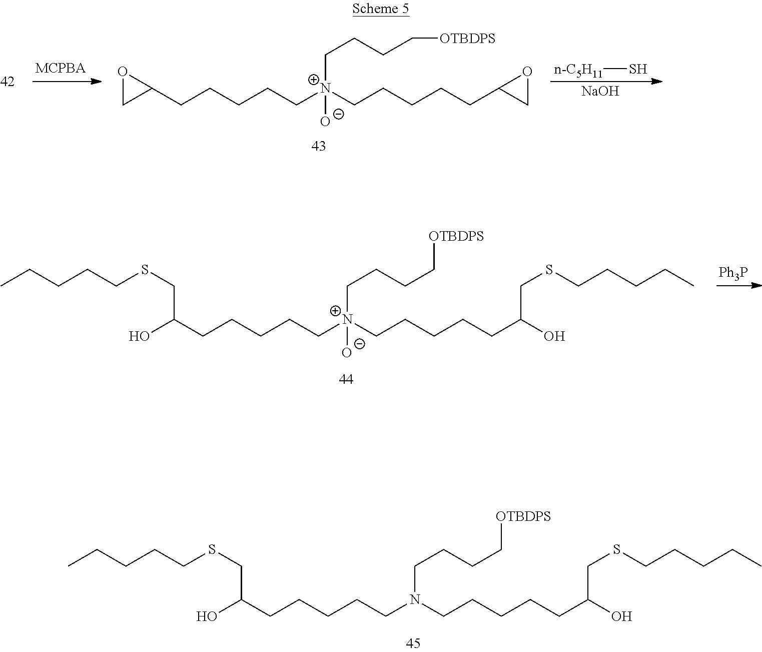

Compounds such as 45 can be converted into lipids such as 5 by esterification of the OH groups with a carboxylic acid in the presence of a condensing agent, for example, a carbodiimide such as EDCI, optionally in the presence of a catalyst such as 4-dimethulaminopyridine (DMAP), followed by release of the TBDPS group with a source of fluoride ion, for instance, pyridine-HF complex. In the case of 5, the carboxylic acid is decanoic acid (Scheme 6).

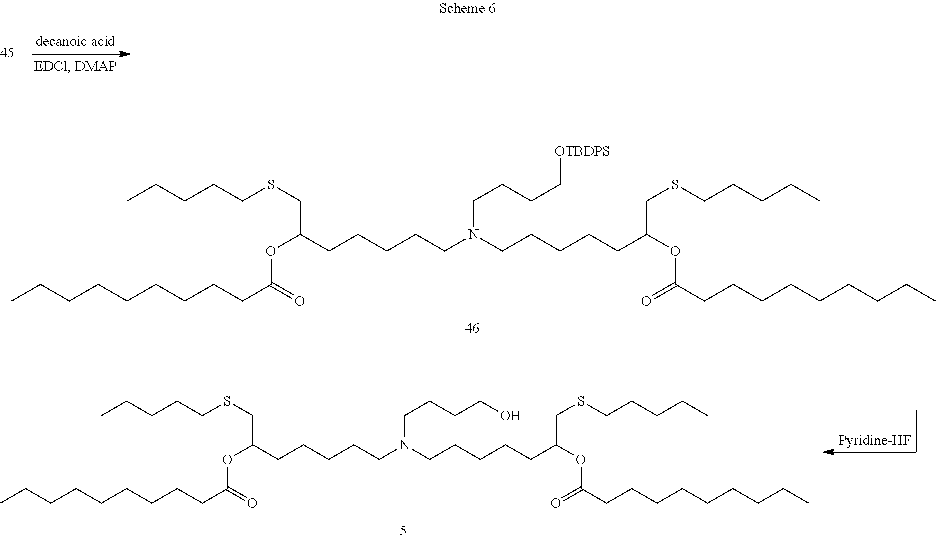

The person skilled in the art will appreciate that the ionizable head group present in lipids 6-32 of Table 1 can be introduced starting with a precursor of said lipids, wherein a ketone functionality is present in lieu of the ionizable head group. The ketone can then be transformed into a suitable ionizable head group by appropriate organic synthesis steps. Therefore, the synthesis of 6-32 starts with the preparation of an appropriate ketone.

Representative, but non-limiting, lipids 6-32 can be made from ketones having the general structure shown as 50 in Scheme 7. Certain steps of the synthesis of a ketone such as 50 are described in detail in co-pending and co-owned WO 2022/246555, incorporated herein by reference. As described in said disclosure, one such step entails subjecting an appropriate ester to Claisen condensation under Mukaiyama conditions, followed by hydrolysis of the resulting beta-ketoester and decarboxylation of the intermediate beta-ketoacid, leading to the formation of a ketone. In certain embodiments, these steps are most advantageously carried out in a “one-pot operation”, meaning that the various synthetic intermediates, while isolable, need not be isolated. For example, ketone 50 can be made starting with the Claisen-Mukaiyama condensation of ester 47, and the synthetic intermediates that optionally need not be isolated are 48 and 49.

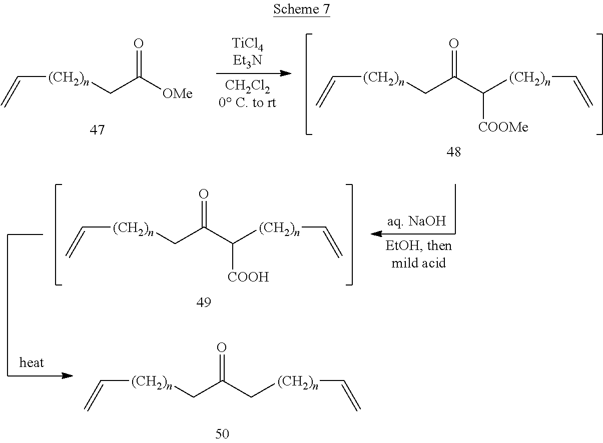

Alternatively, a ketone such as 50 and its congeners can be prepared by certain synthetic steps that are described in detail in co-owned and co-pending U.S. provisional patent application No. 63/445,854, incorporated herein by reference. One such step entails the double alkylation of a reagent such as tosyl methyl isocyanide (TosMIC) by reaction with about two equivalents of an alkyl halide (chloride, bromide or iodide) or sulfonate (tosylate, mesylate, triflate, and the like) under basic conditions, followed by acidic hydrolysis of the product. This is exemplified in Scheme 8 with the synthesis of 50 by the TosMIC method.

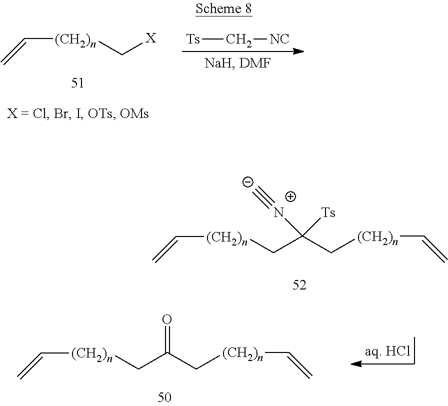

A ketone such as 50 is symmetrical, meaning that the alkyl groups bonded to the carbonyl are identical. The above Provisional Application teaches that unsymmetrical congeners of 50, wherein two different alkyl groups are bonded to the carbonyl group, can be prepared by sequential alkylation of TosMIC with two different alkyl halides or sulfonates, followed by hydrolysis of the product under acidic conditions (Scheme 9).

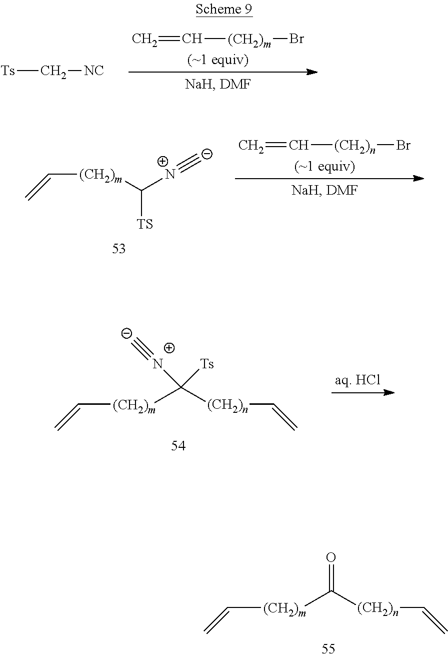

The conversion of a ketone such as 50 into a lipid of Formula A starts with the epoxidation of the double bonds with a suitable epoxidizing reagent. Without intending to be limiting, appropriate reagents include peroxy-carboxylic acids, such as magnesium monoperoxyphthalate, performic acid, peracetic acid, meta-chloroperoxybenzoic acid (MCPBA), and the like, which convert the double bonds directly into epoxides, or sources of electrophilic halogen, such as chlorine, bromine, iodine, N-chlorosuccinimide (NCS), N-bromosuccinimide (NBS), N-iodosuccinimide (NIS), and the like, in aqueous medium, which convert the double bonds into halohydrins that can be transformed into epoxide by base treatment. In either case, the result is the formation of bis-epoxide xx as a mixture of oxirane diastereomers, which are not separated. This is illustrated in Scheme 10 with the formation of 56 by treatment of 50 with MCPBA. Subsequent regioselective opening of the epoxides with a thiol, R—SH, under basic conditions produces dihydroxyketone 57.

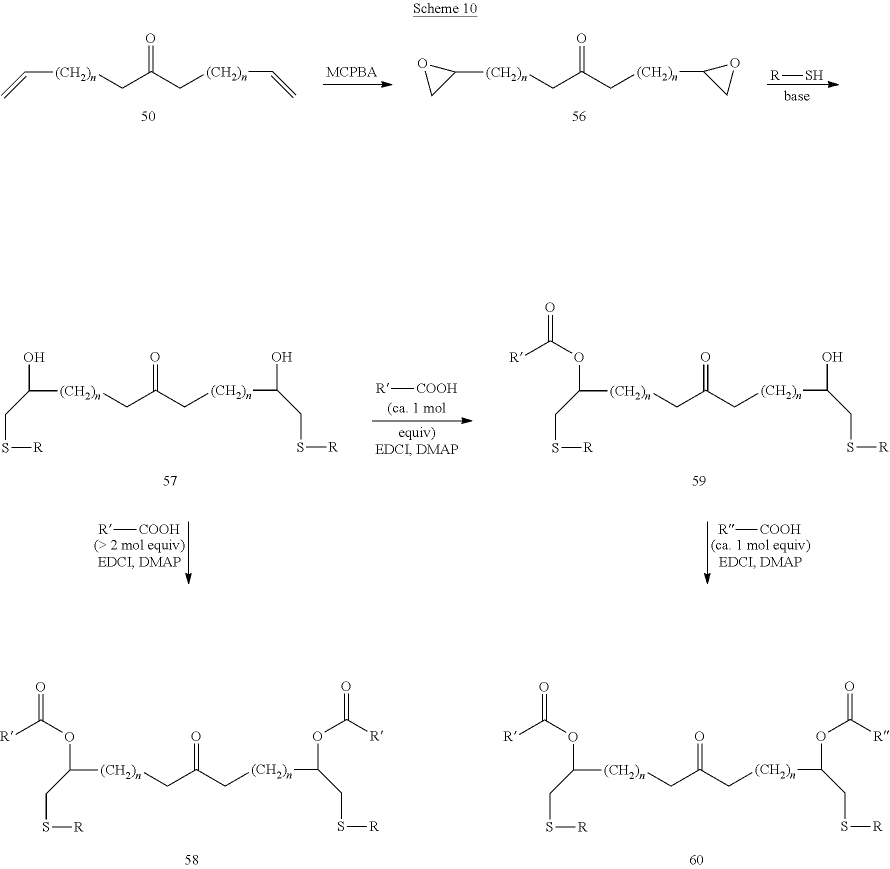

The dihydroxyketone is then esterified with appropriate carboxylic acids. Depending on conditions, the esterification reaction can be carried out so that symmetrical diester 58 is formed as the major product, for example, by the use of at least 2 molar equivalents of an acid R′—COOH in the presence of a condensing agent, for example, a carbodiimide such as EDCI, and DMAP, or so that monoester 59 is formed as the major product, for example, by the use of about 1 molar equivalent of an acid R′—COOH in the presence of a condensing agent, for example, a carbodiimide such as EDCI, and DMAP. Furthermore, monoester 59 can be converted into unsymmetrical diester 60 by subsequent reaction with a second carboxylic acid, R″—COOH, in the presence of a condensing agent, for example, a carbodiimide such as EDCI, and DMAP (Scheme 10). The ketone group in 58 or 60 can then be transformed into any ionizable head group of type 1-9 (see definitions above) by chemical methods that are well known to those of skill in the art. Representative, but by no means limiting, examples are set forth below.

The ketone of type 50 required for the synthesis of lipid 6 is 61 (a variant of 50 with n=3), the diepoxy ketone obtained from 61 is 62 (a variant of 56 with n=3), the thiol utilized in the epoxide opening reaction is cyclohexanethiol, and the acid utilized in the esterification of the resulting dihydroxyketone 63 is decanoic acid (Scheme 11). The ketone in product 64 is then transformed into a type 1 ionizable head group by selective reduction with a hydride reagent, for example, sodium borohydride in an appropriate solvent, for example, an alcohol such as ethanol or isopropanol, followed by esterification of the resulting alcohol 65 with 4-(dimethylamino) butanoic acid, or a salt thereof such as the corresponding hydrochloride, in the presence of a condensing agent, for example, a carbodiimide such as EDCI, and DMAP, resulting in formation of lipid 6.

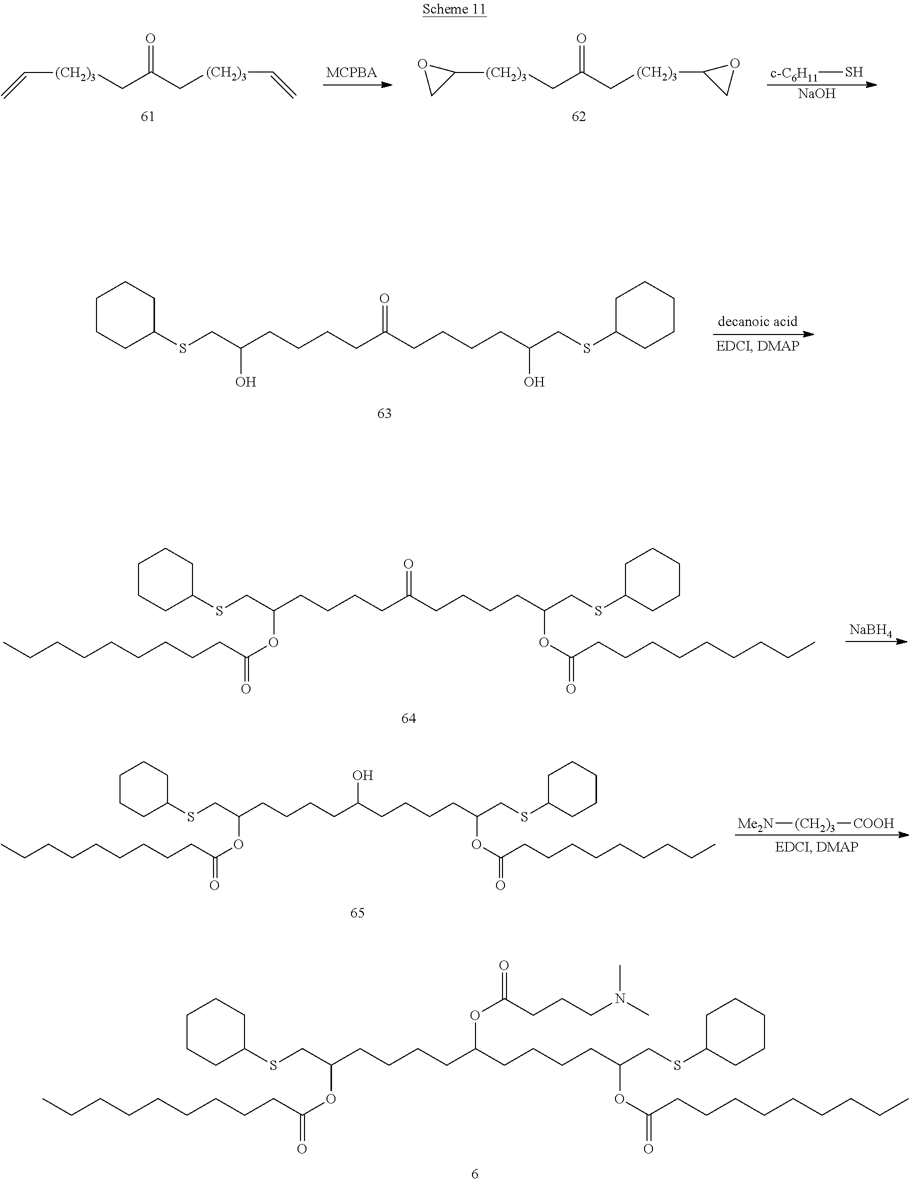

The ketone of type 50 required for the synthesis of lipid 7 is 66 (a variant of 50 with n=4), the diepoxy ketone obtained from 66 is 67 (a variant of 56 with n=4), the thiol utilized in the epoxide opening reaction is 1-hexanethiol, and the acid utilized in the esterification of the resulting dihydroxyketone 68 is decanoic acid (Scheme 12). The ketone in product 69 is then transformed into a type 1 ionizable head group as shown in Scheme 11 above.

The synthesis of lipid & (Scheme 13) involves the esterification of compound 68 of Scheme 12 with 3-cyclohexylpropanoic acid, followed by the conversion of the ketone in the resulting 71 into a type 1 ionizable head group as shown in Scheme 11 above.

The synthesis of lipid 9 requires acid 74, which can be made, for example, by the method shown

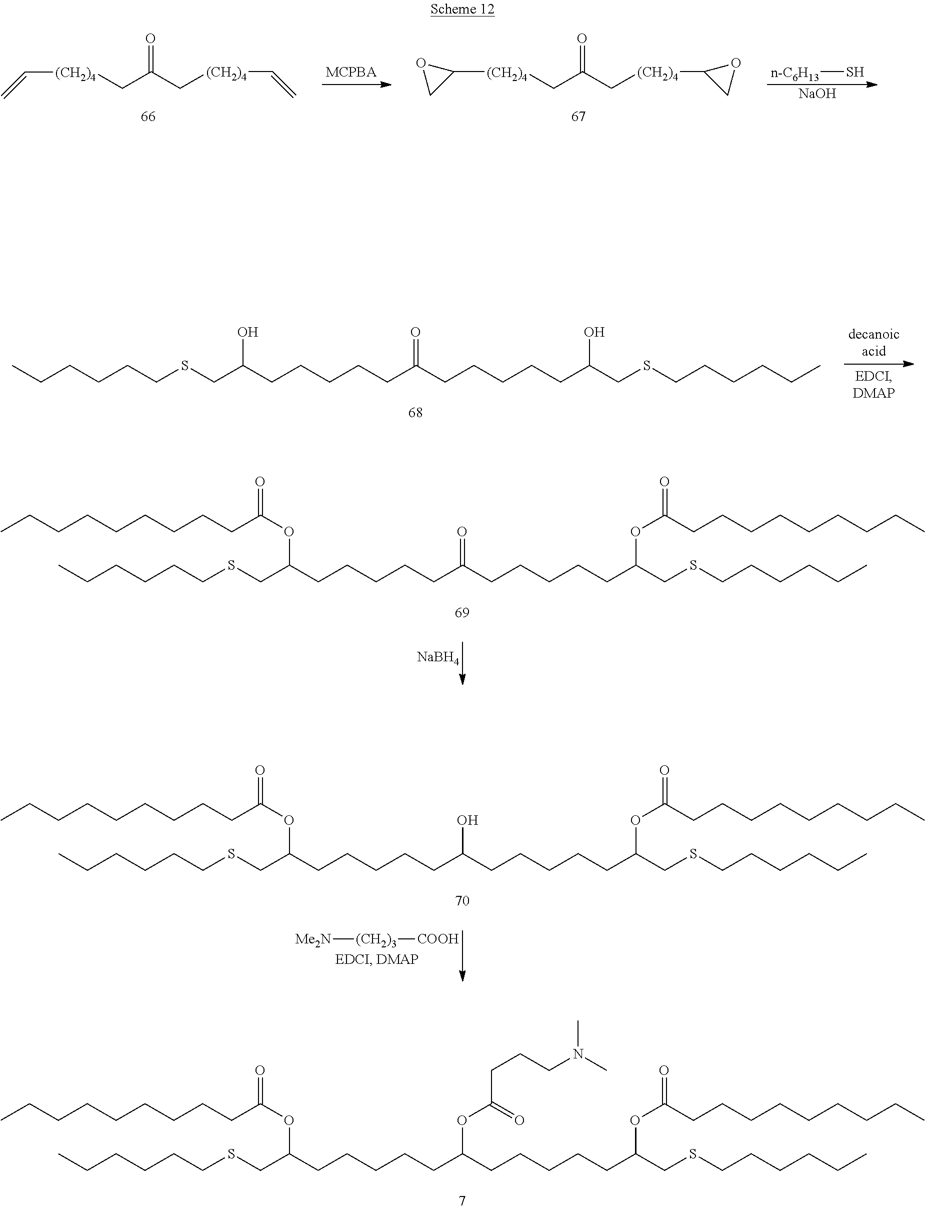

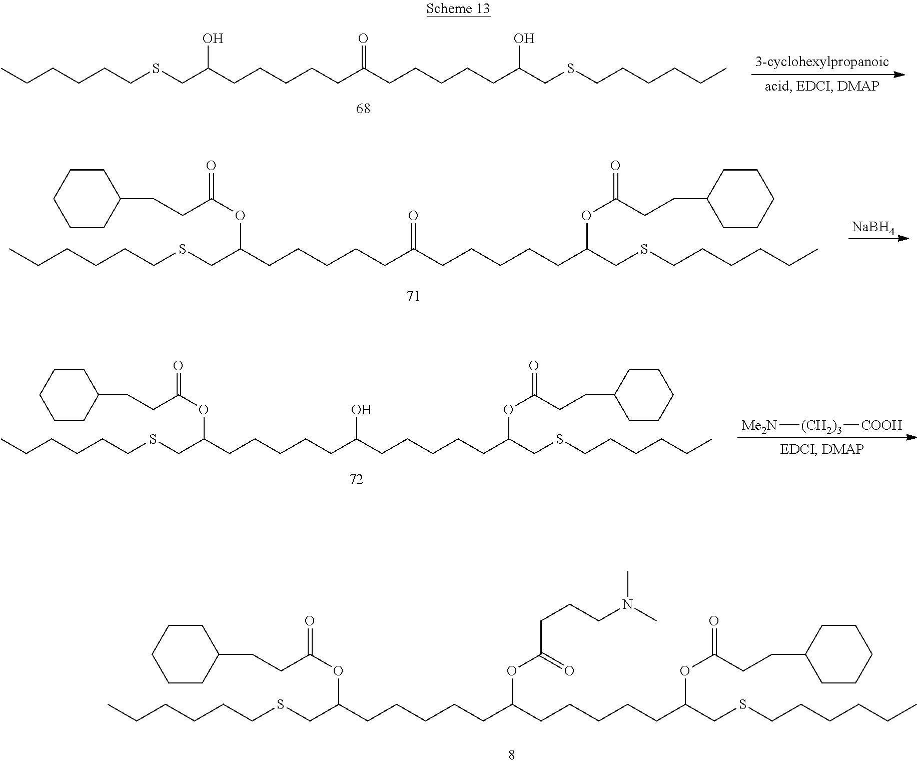

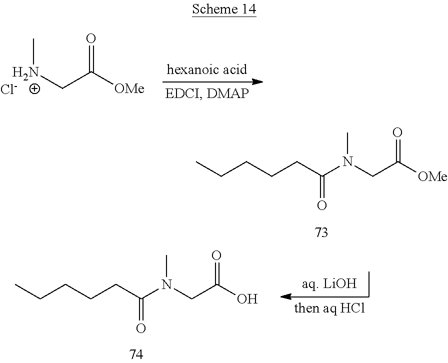

in Scheme 14, details of which are provided in co-owned and U.S. provisional patent application No. 63/410,273. Mono-esterification of dihydroxyketone 68 with decanoic acid and further esterification of the resulting 75 with acid 74 gives 76. the conversion of the ketone in 76 into a type 1 ionizable head group is then accomplished as shown in Scheme 11 above (Scheme 15).

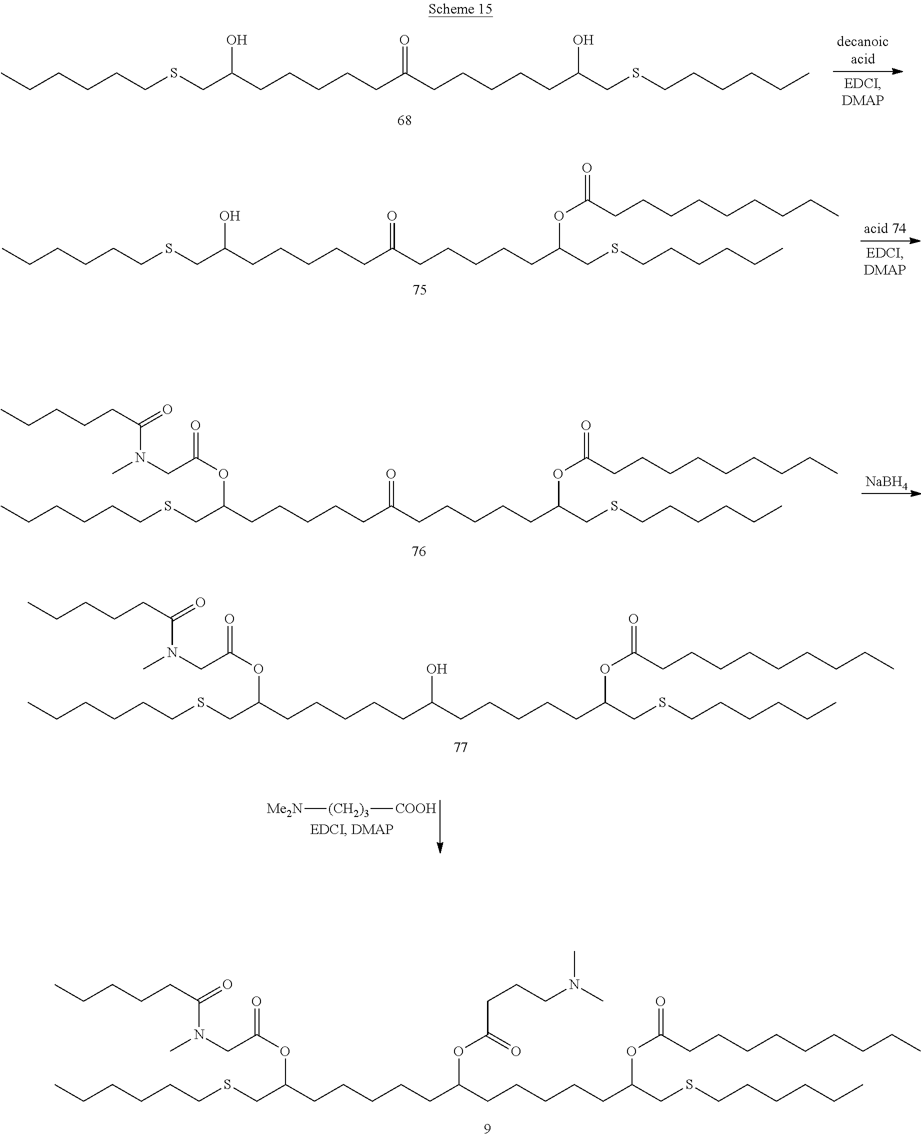

The diepoxy ketone required for the synthesis of lipid 10 is 67, the thiol utilized in the epoxide opening reaction is cyclohexanethiol, and the acid utilized in the esterification of the resulting dihydroxyketone 78 is decanoic acid (Scheme 16). The ketone in product 79 is then transformed into a type 1 ionizable head group as shown in Scheme 11 above.

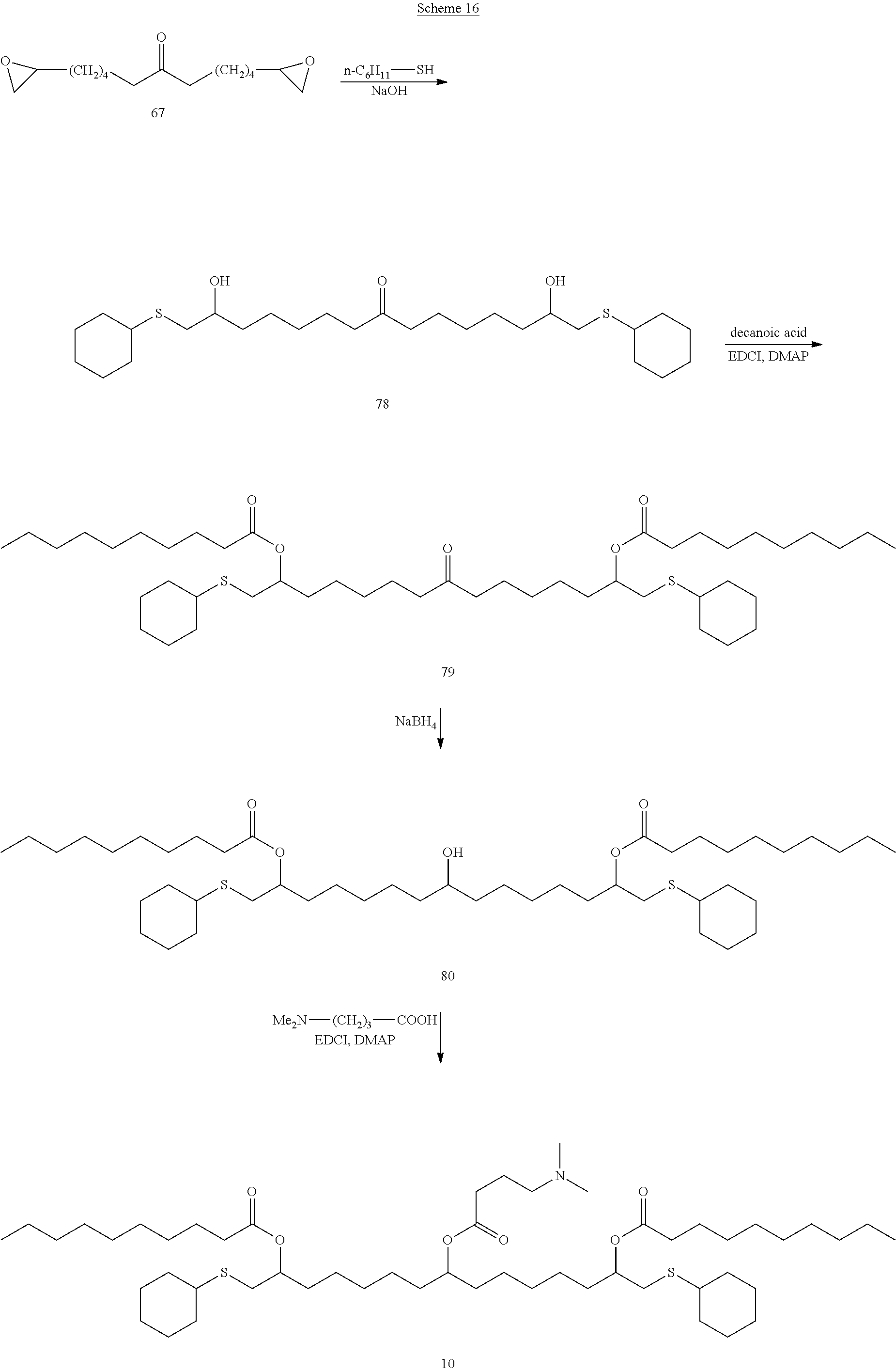

The synthesis of lipid 11 (Scheme 17) involves the esterification of dihydroxyketone 78 with nonanoic acid, followed by conversion of the ketone in the resulting diester 81 into a type 1 ionizable head group as shown in Scheme 11 above.

The synthesis of lipid 12 (Scheme 18) starts with diepoxy ketone 62. The thiol utilized in the epoxide opening reaction is 1-pentanethiol, and the acid utilized in the esterification of the resulting dihydroxyketone 83 is decanoic acid. The ketone in product 84 is then transformed into a type 2 ionizable head group, starting with the formation of ketal 85 by reaction with 1,2,4-

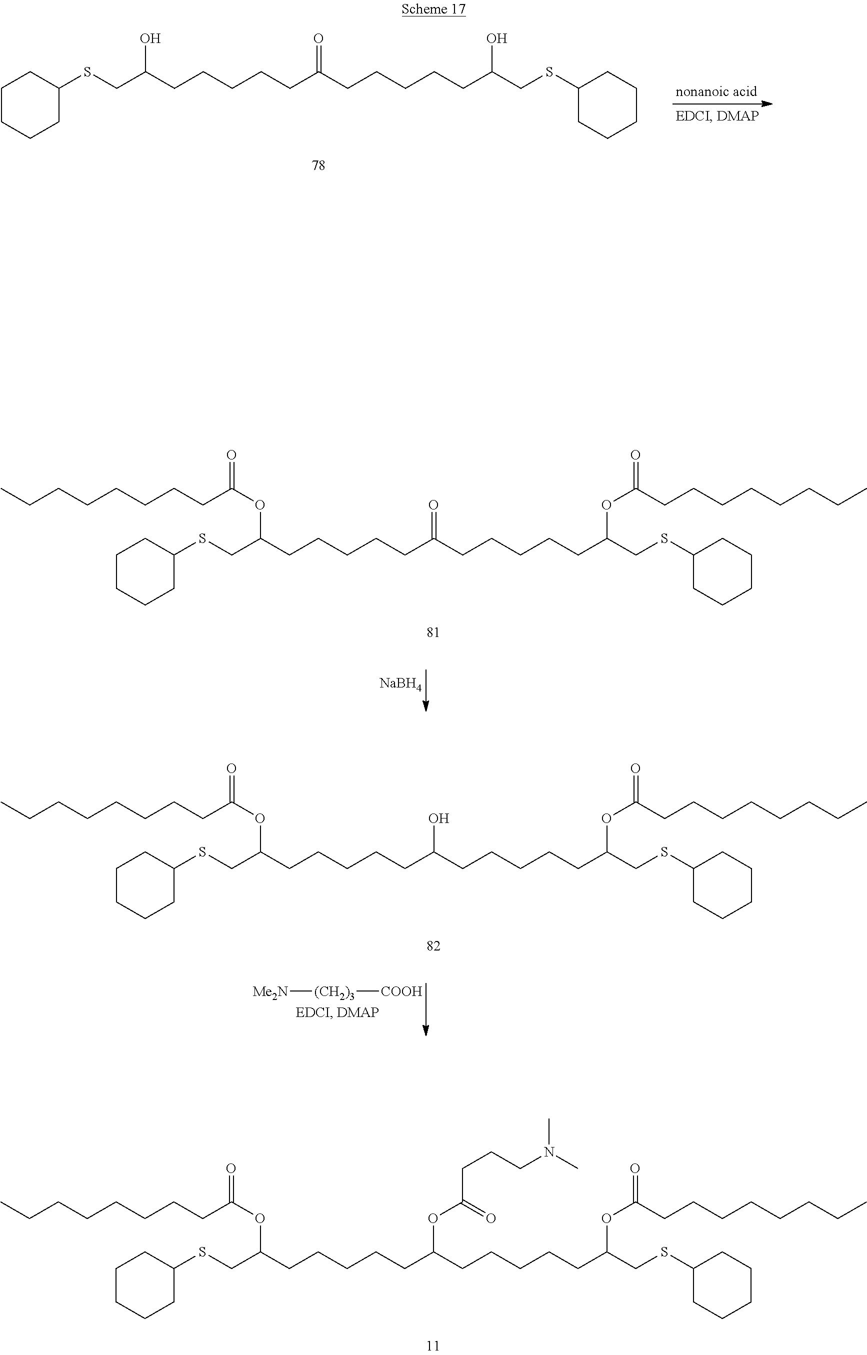

butanetriol in an appropriate solvent, at a suitably elevated temperature, and in the presence of an acid catalyst, preferentially with continuous azeotropic removal of the water formed during the reaction. For example, the reaction can be carried out in refluxing toluene in the presence of pyridinium para-toluenesulfonate (PPTS), and a Dean-Stark trap can be used for water removal. The OH group in the product 85 of the ketalization step is then converted into a leaving group such as a halide (chloride, bromide, or iodide) or a sulfonate ester (tosylate, mesylate, triflate, and the like) in preparation for the introduction of a dimethylamino moiety. For example, 85 can be transformed into tosylate 86 by reaction with para-toluenesulfonyl chloride (TsCl) in an basic solvent such as pyridine, or in a nonbasic one such CH₂Cl₂ the presence of a base such as triethylamine, in either case optionally in the presence of a catalyst such as 4-dimethylaminopyridine. The reaction of tosylate 86 with dimethylamine in an appropriate solvent or mixture of solvents, for example tetrahydrofuran (THF) and methanol, at a suitably elevated temperature, optionally in the presence of a base such as Na₂CO₃ or K₂CO₃, and optionally with microwave activation, produces lipid 12.

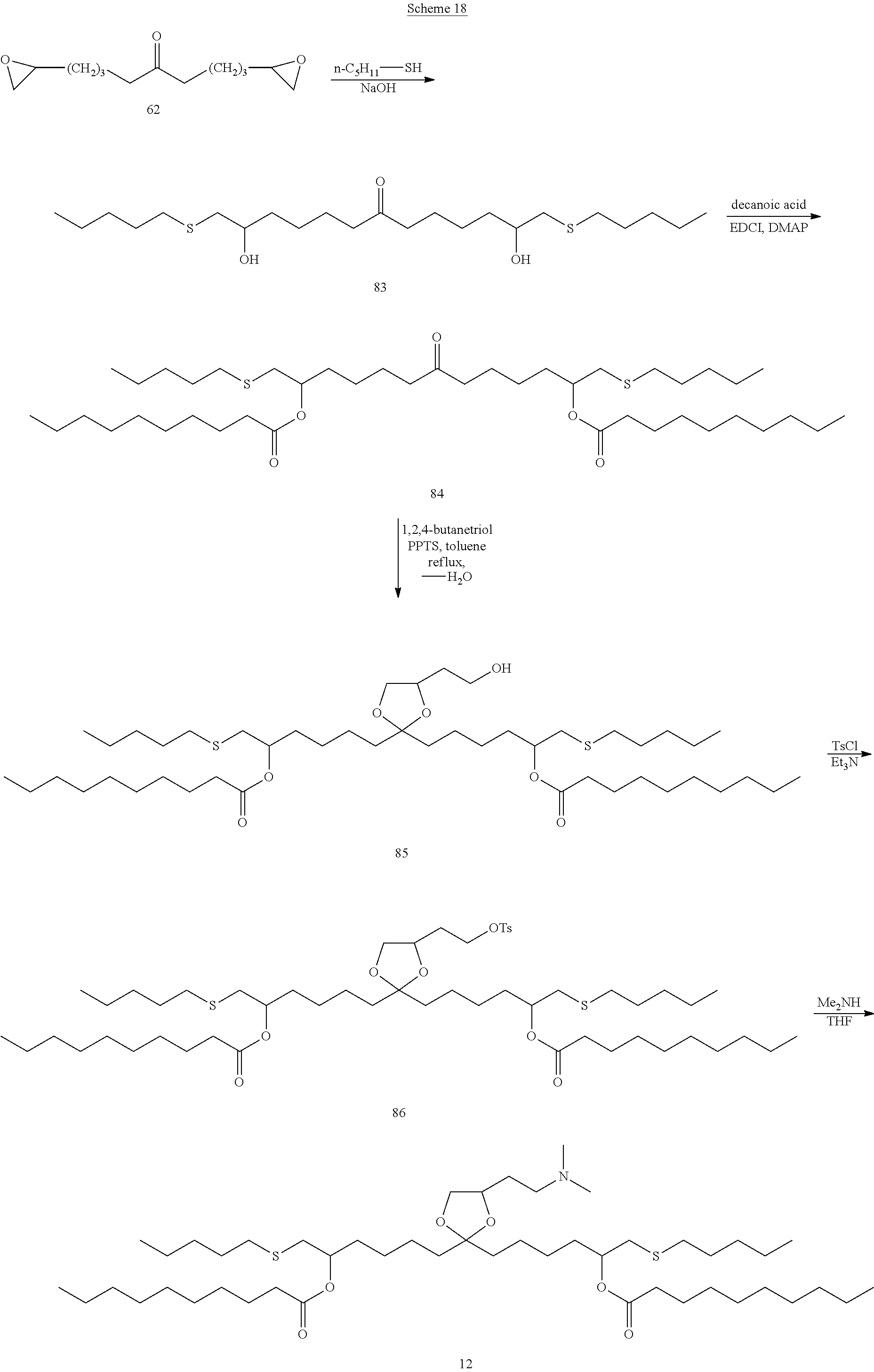

The synthesis of lipid 13 (Scheme 19) demonstrates a method for the conversion of a ketone into a type 4 ionizable head group. Thus, ketone 84 is transformed into bromoketal 87 by reaction with 3-bromo-1,2-propanediol in an appropriate solvent, at a suitably elevated temperature, and in the presence of an acid catalyst, preferentially with continuous azeotropic removal of the water formed during the reaction. For example, the reaction can be carried out in refluxing toluene in the presence of pyridinium para-toluenesulfonate (PPTS), and a Dean-Stark trap can be used for water removal. Reaction of 87 with 4-methylamino-1-butanol in an appropriate solvent or mixture of solvents, for example in acetonitrile, at a suitably elevated temperature, optionally in the presence of a base such as Na₂CO₃ or K₂CO₃, and optionally with microwave activation, produces 13.

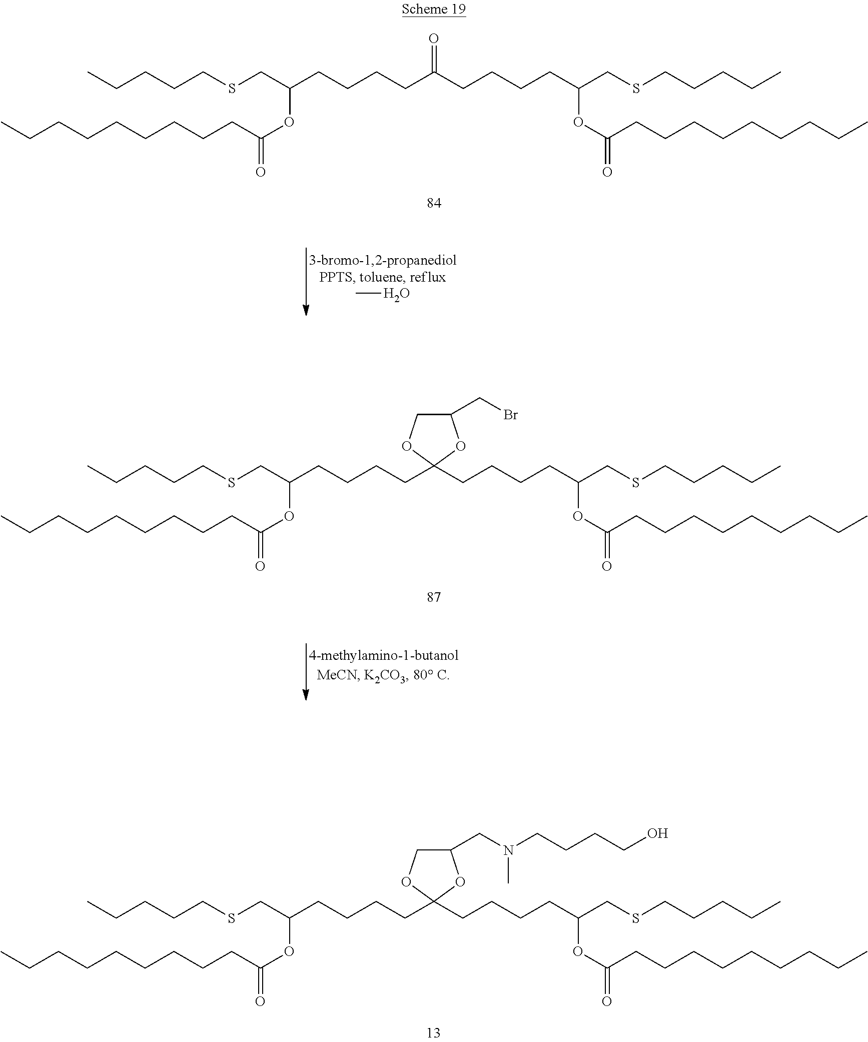

Lipids 14 and 15 can be made in a similar manner from tosylate 86 by reaction with 4-ethylamino-1-butanol (Scheme 20) and piperidin-4-ol (Scheme 21), respectively.

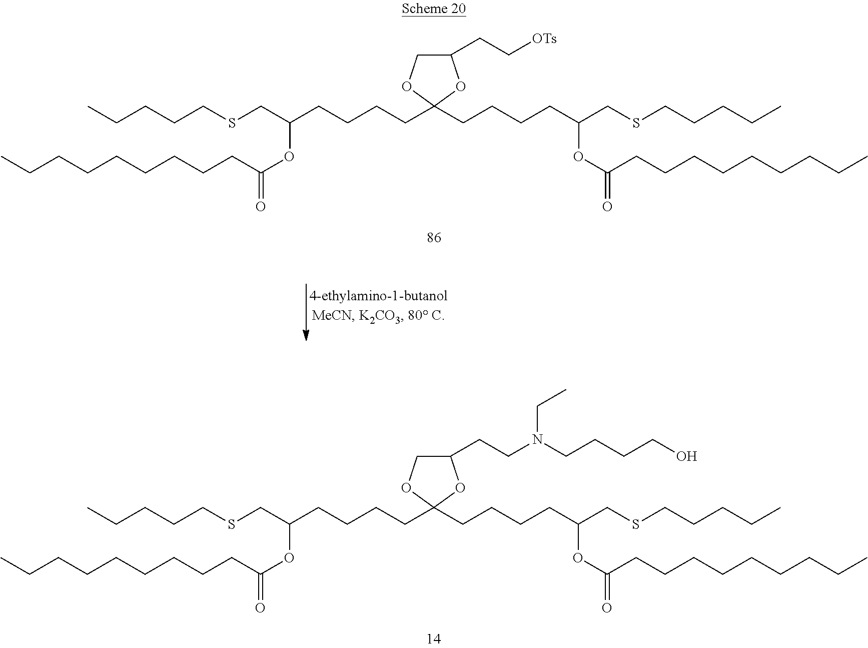

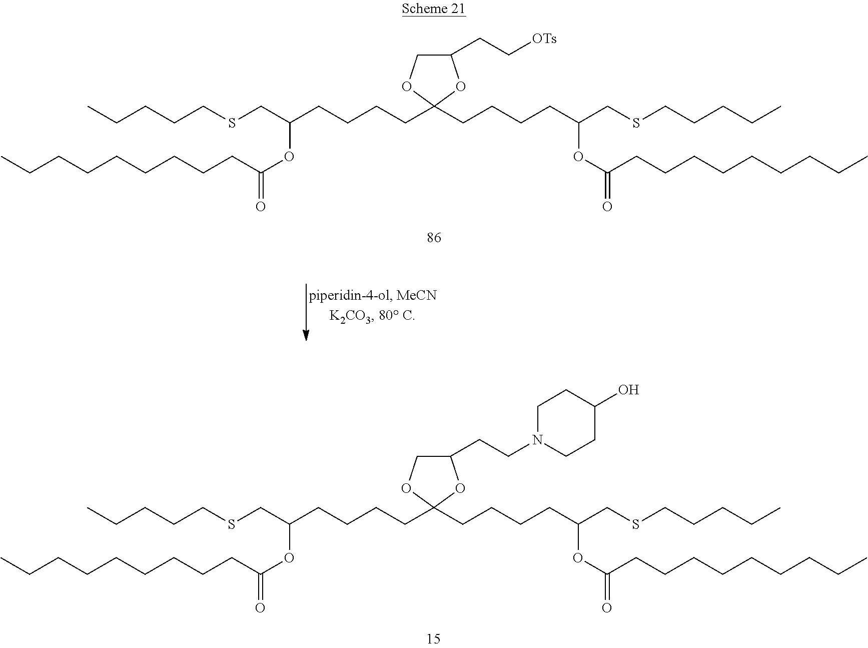

Lipid 16 can be made from ketone 64 of Scheme 11 by conversion of the carbonyl group into a type 2 ionizable head group by the method outlined in Scheme 18 (Scheme 22).

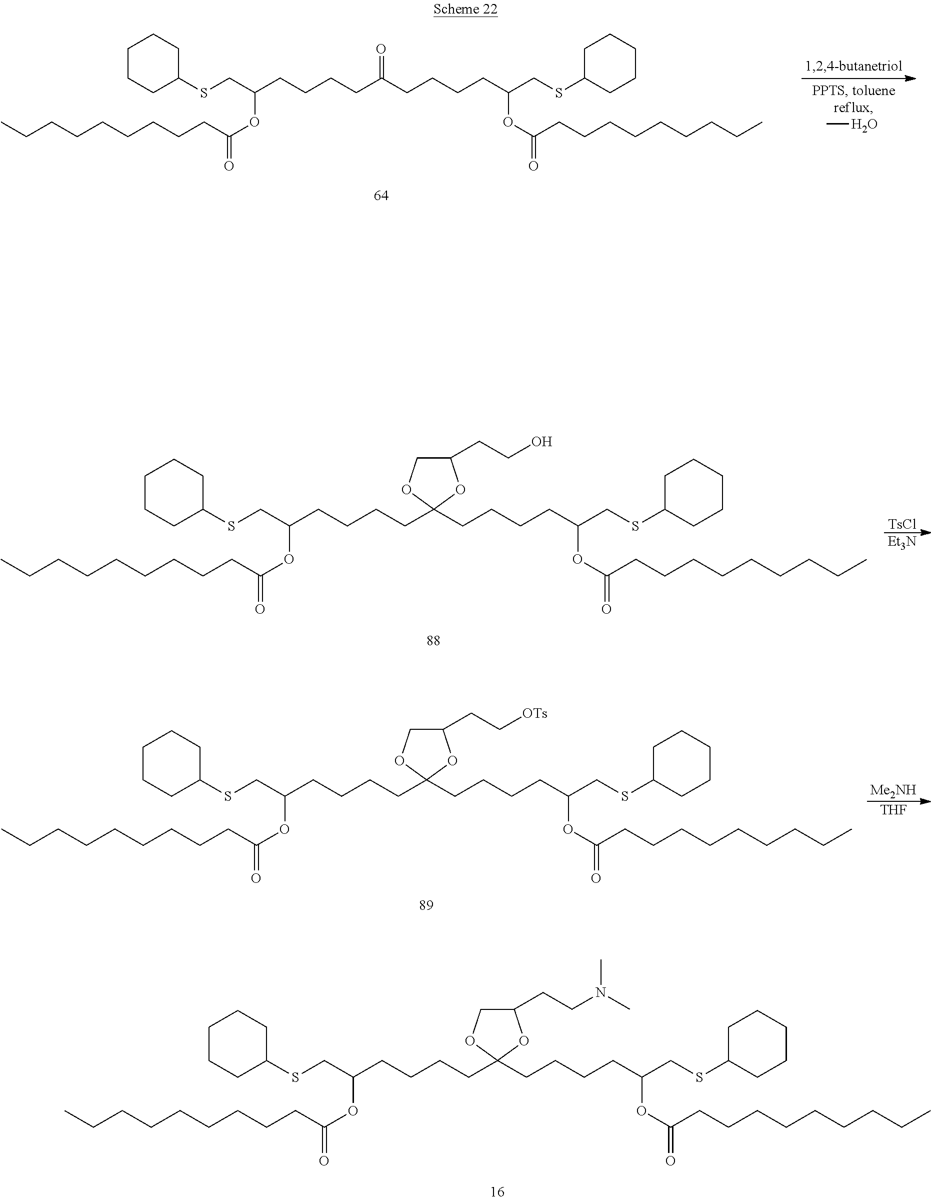

The head group present in lipids 17 and 18 is a variant of a type 3 ionizable group, and it can be created starting with the conversion of ketone 64 of Scheme 11 into ketal 90 by reaction with 2,2-bis(hydroxymethyl) propane-1,3-diol (pentaerythritol) in an appropriate solvent, at a suitably elevated temperature, and in the presence of an acid catalyst, preferentially with continuous azeotropic removal of the water formed during the reaction. For example, the reaction can be carried out in refluxing toluene in the presence of pyridinium para-toluenesulfonate (PPTS), and a Dean-Stark trap can be used for water removal. Compound 90 is then mono-esterified with 3-(dimethylamino) propanoic acid or its hydrochloride salt, for example, in the presence of a carbodiimide such as EDCI and optionally DMAP, to give 17. The same esterification reaction carried out with 4-(dimethylamino) butanoic acid or its HCl salt produces 18 (Scheme 23).

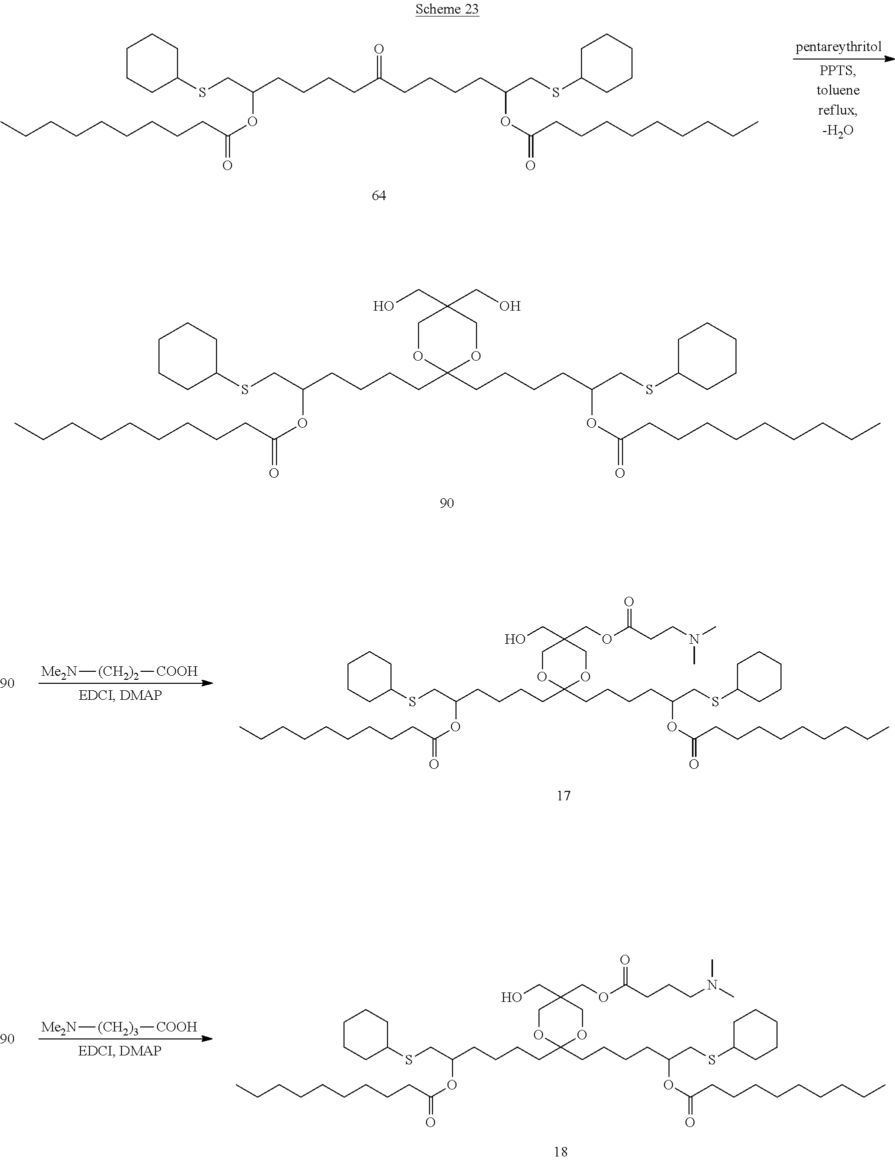

The synthesis of lipid 19 (Scheme 24) involves the conversion of the ketone compound 69 of Scheme 12 into a type 2 ionizable head group by a method similar to that shown in Scheme 19, except that dimethylamine is used in the final reaction.

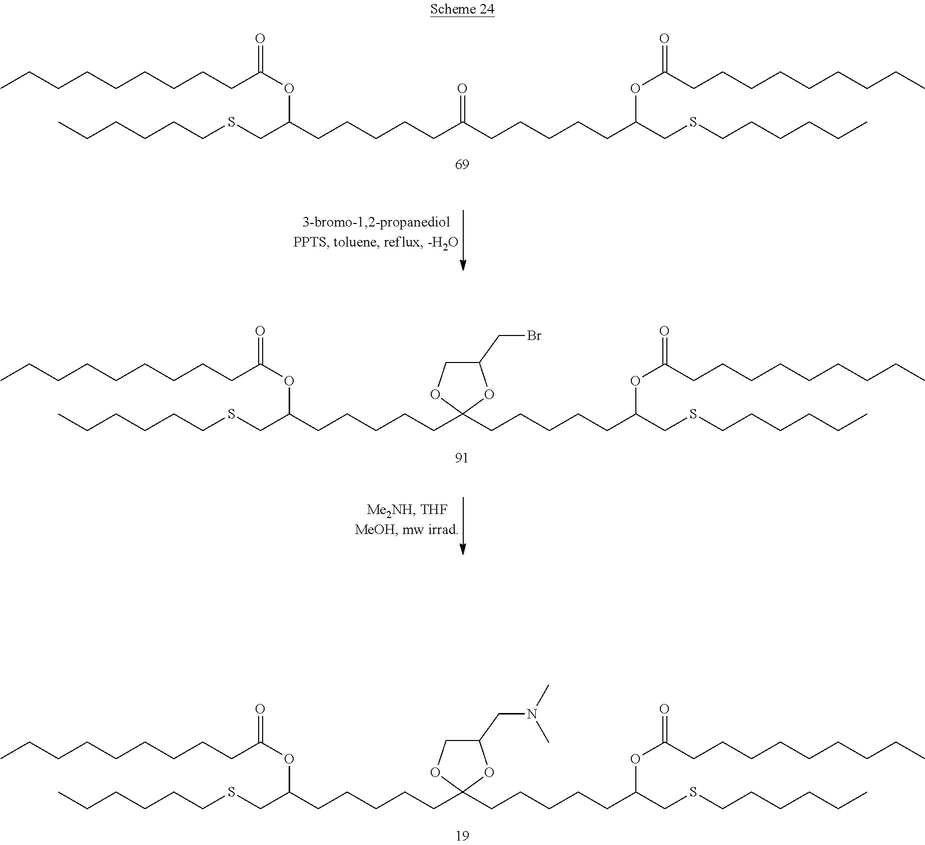

Lipid 20 (Scheme 25) can be made by reaction of bromoketal 91 with 4-(methylamino)-1-butanol by the method shown earlier in Scheme 19.

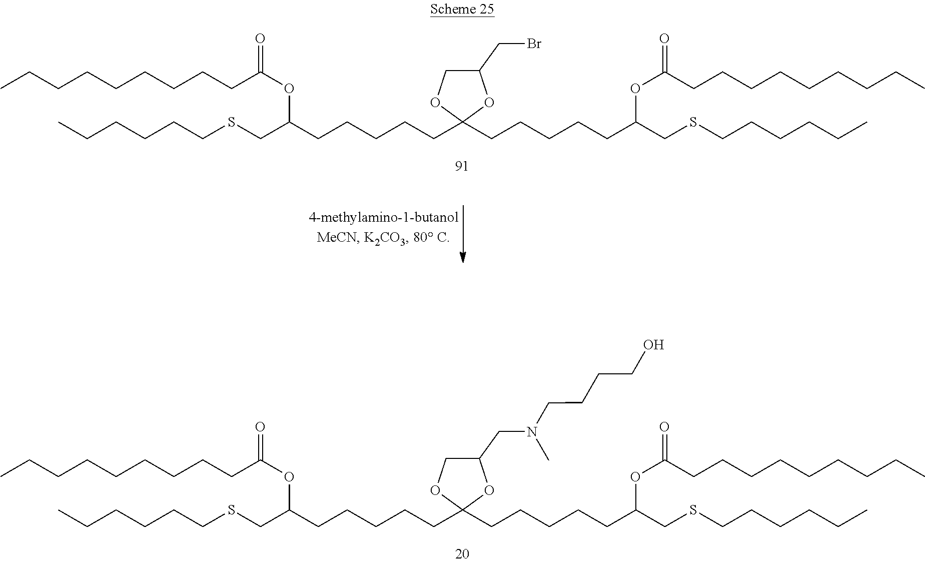

The type 4-like ionizable group of lipid 21 can be introduced by conversion of ketone 69 into ketal 92, followed by tosylation and displacement with 4-methylamino-1-butanol (Scheme 26).

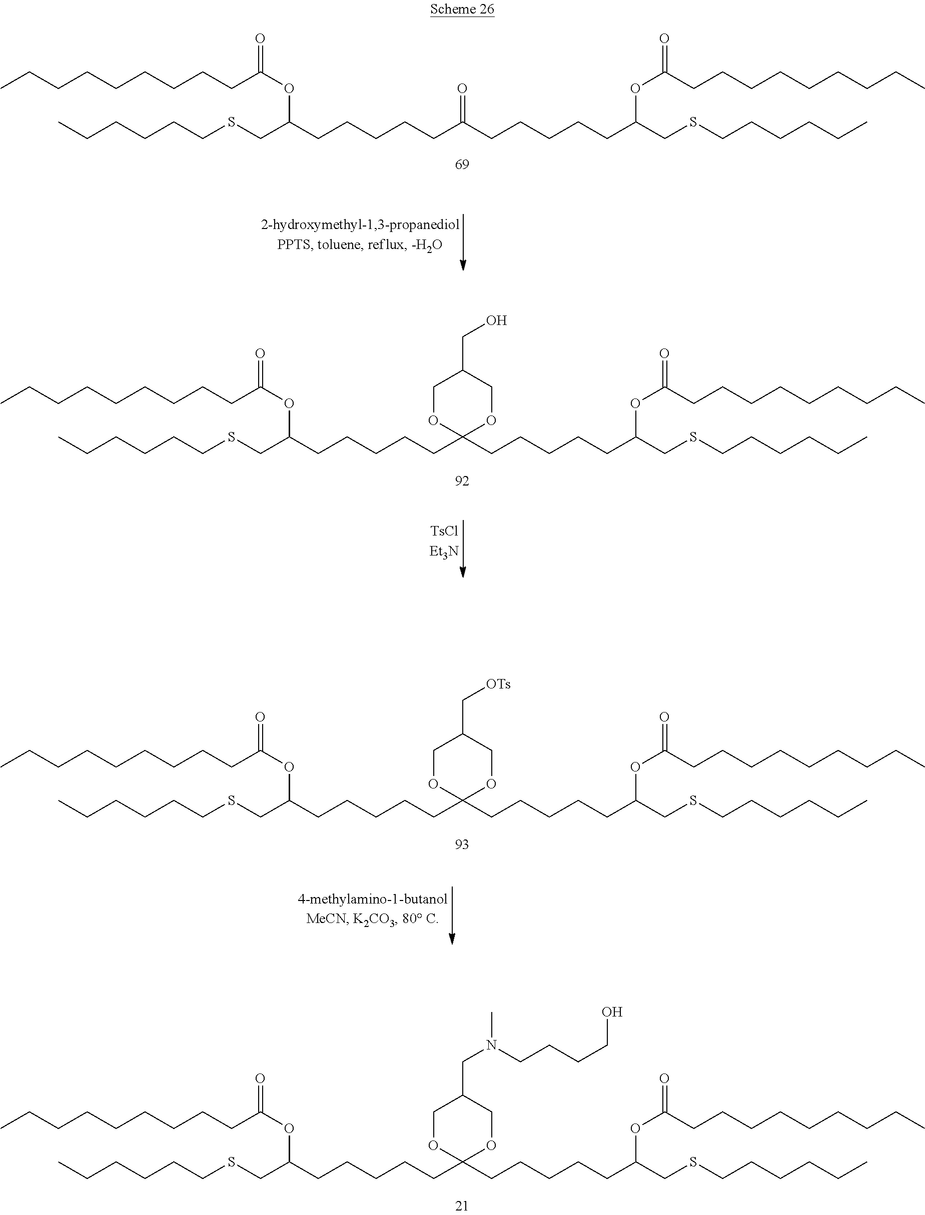

The synthesis of lipids 22-32 demonstrates a method for the conversion of a ketone group into a type 7 ionizable head. Thus, lipid 22 can be made from ketone 84 of Scheme 18 starting with reductive amination with an O-protected form of 4-amino-1-butanol, for example, a silyl ether such as a tert-butyldiphenylsilyl (TBDPS) ether, in an appropriate solvent, for example, 1,2-dichloroethane, in the presence of a reducing agent, for example, a borohydride reagent such as sodium triacetoxyborohydride, sodium cyanobrohydride, and the like, and optionally in the presence of a catalyst such as acetic acid (Scheme 27). A secondary amine such as 94 thus formed is then N-alkylated to produce a tertiary amine. In the case of lipid 22, this entails an N-methylation to give 95. This can be accomplished either by treatment of 94 with aqueous formaldehyde and a reducing agent such as, but not limited to, sodium triacetoxyborohydride, in an appropriate solvent such as THE, or with a methylating agent such as a methyl halide (chloride, bromide, iodide), sulfate, sulfonate, sulfonium, or sulfoxonium reagent, also in an appropriate solvent and under suitable conditions. Lipid 22 is obtained upon release of the TBDPS group in 95 with a source of fluoride ion, for example, HF-pyridine complex.

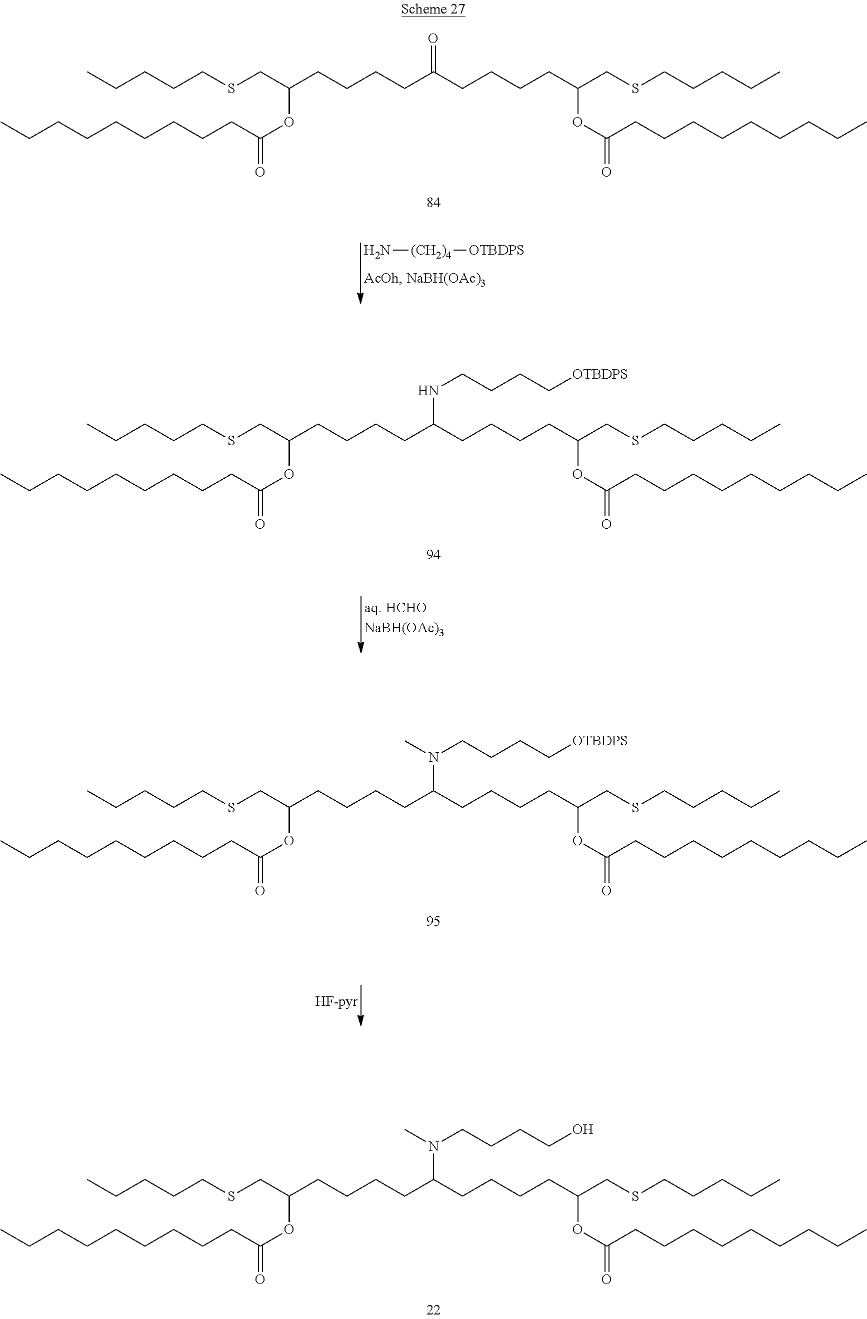

The synthesis of lipid 23 (Scheme 28) starts with the reaction of bis-epoxy ketone 62 of Scheme 11 with 1-heptanethiol and esterification of the resulting 96 with decanoic acid to produce 97. The latter compound is then converted into lipid 23 by the same method shown in Scheme 27 above.

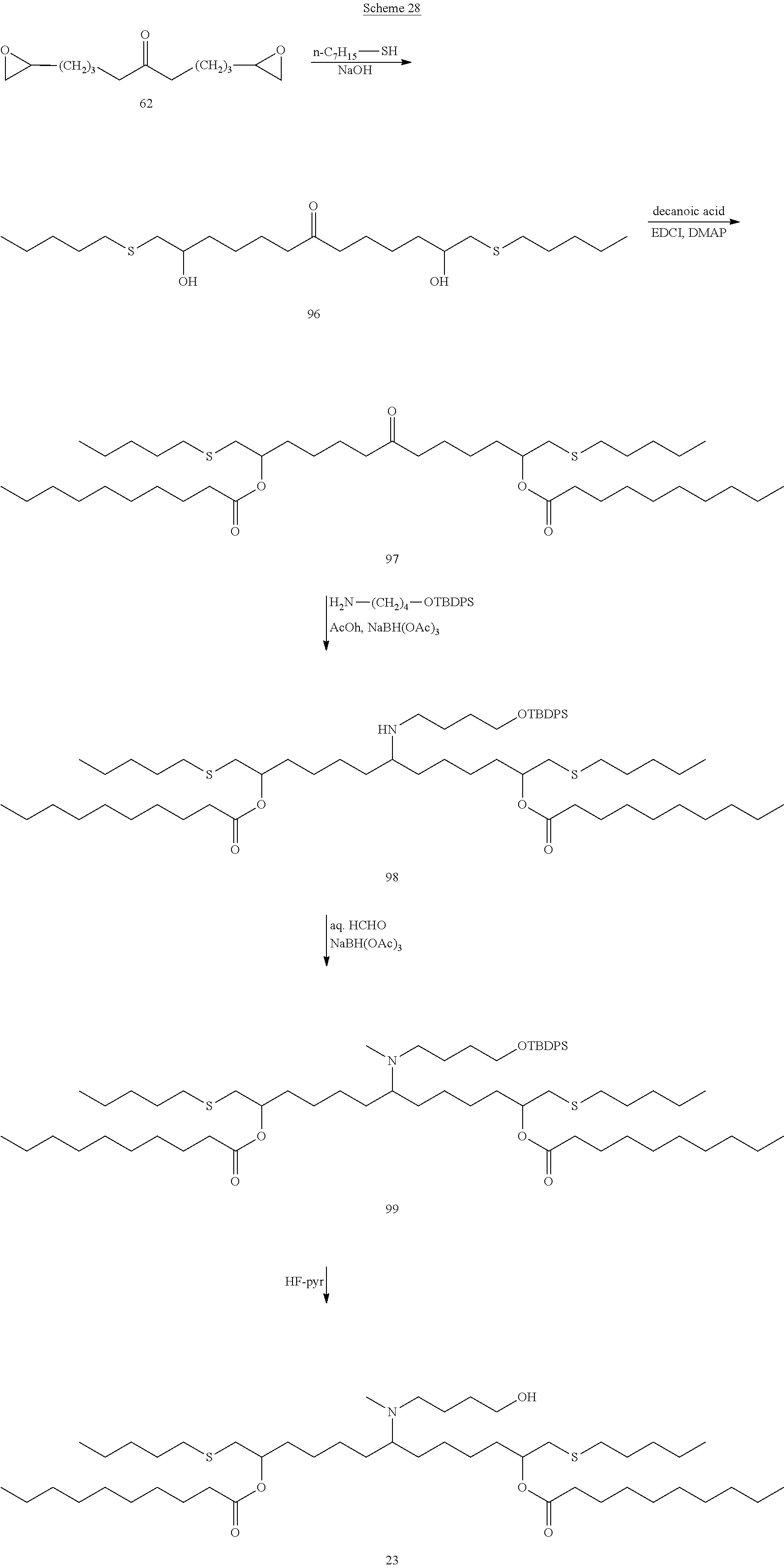

The synthesis of lipid 24 (Scheme 29) can be achieved in a like manner, except that compound 96 is esterified with nonanoic acid.

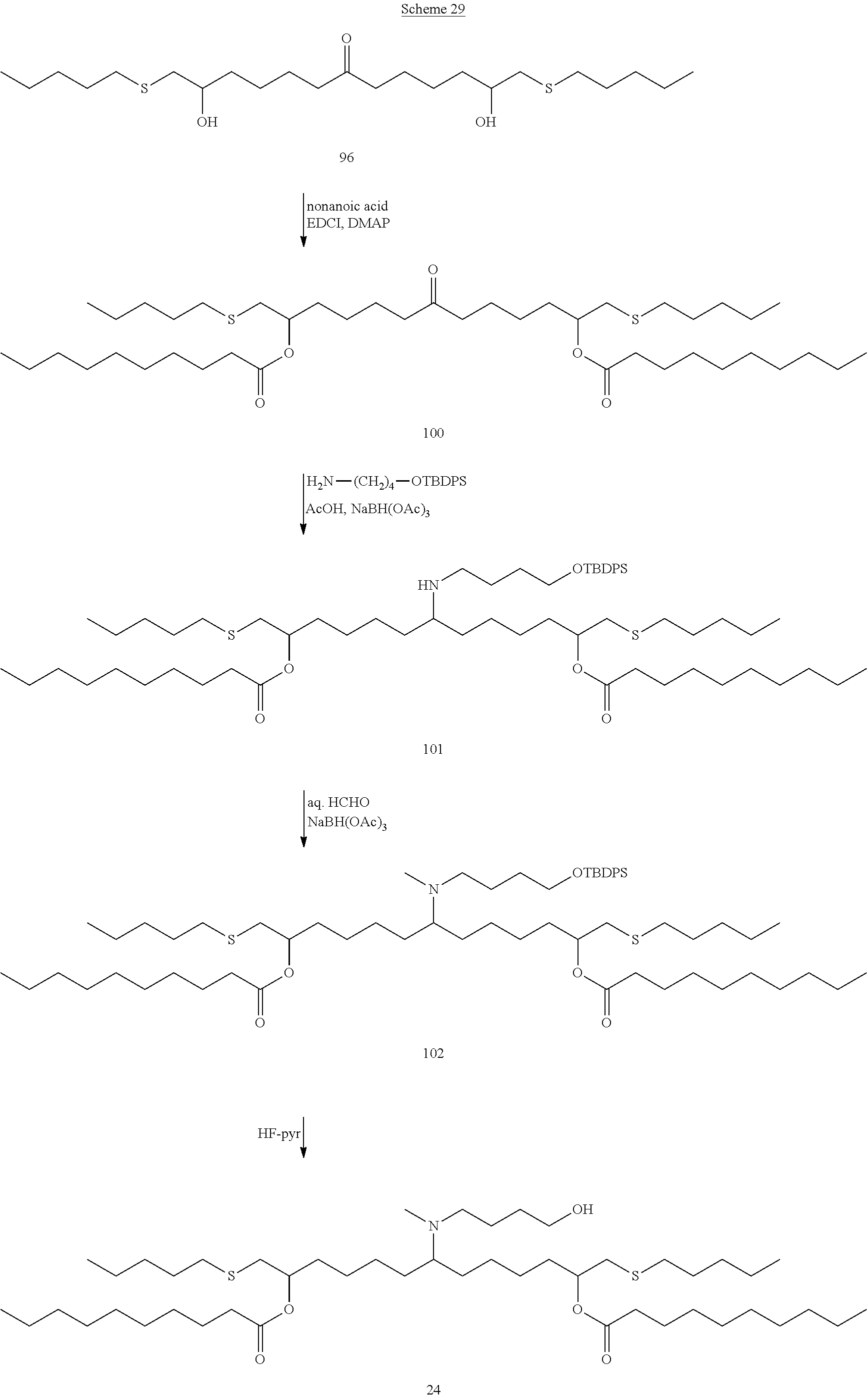

Lipid 25 can be prepared starting with the reaction of bis-epoxy ketone 67 of Scheme 12 with 1-pentanethiol, followed by esterification of the resulting 103 with nonanoic acid (Scheme 30). Ketone 104 thus obtained can then be transformed into lipid 25 by the method shown in Scheme 27 above.

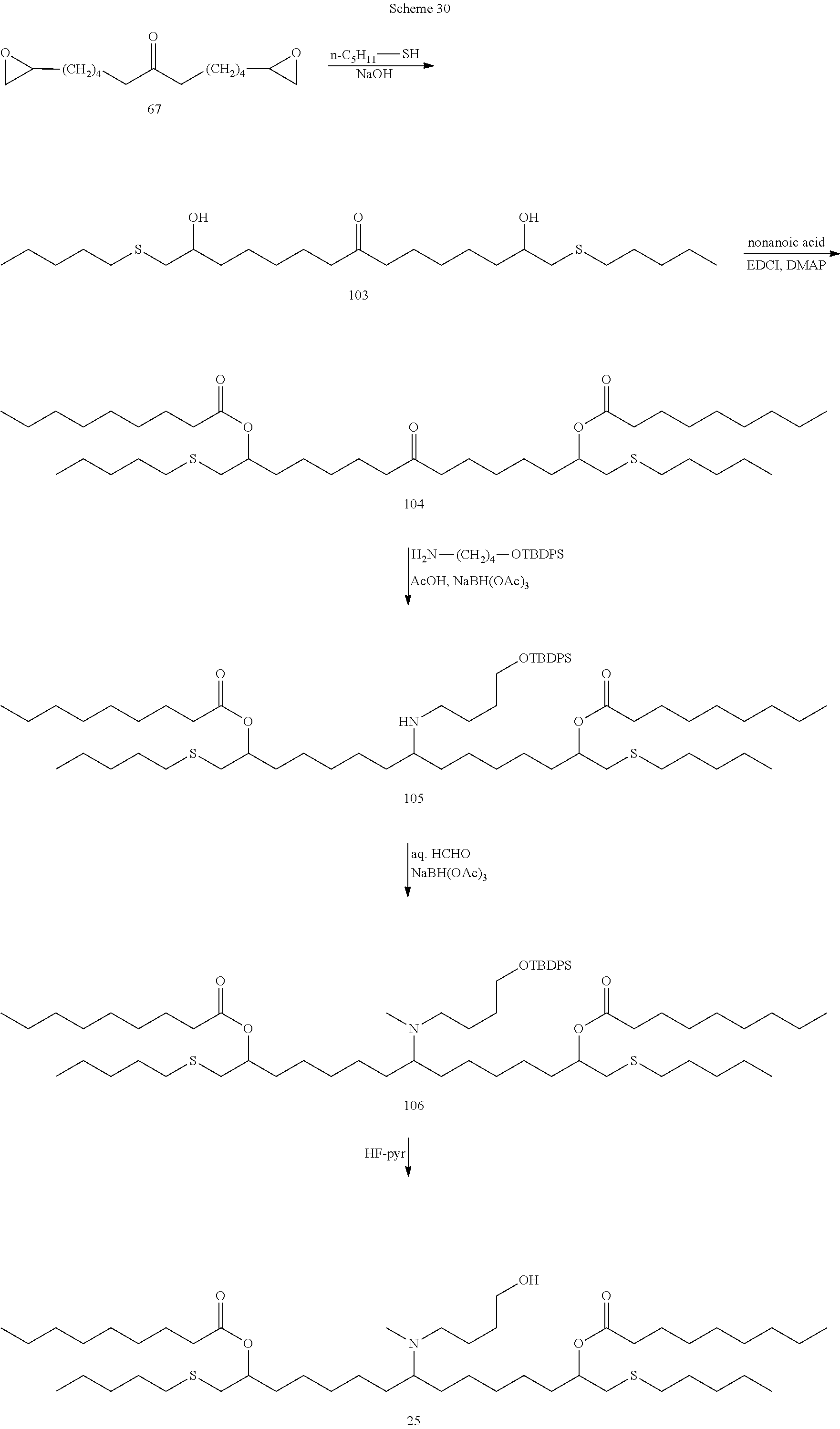

Lipid 26 can be made in a like manner starting with the esterification of compound 103 with decanoic acid (Scheme 31).

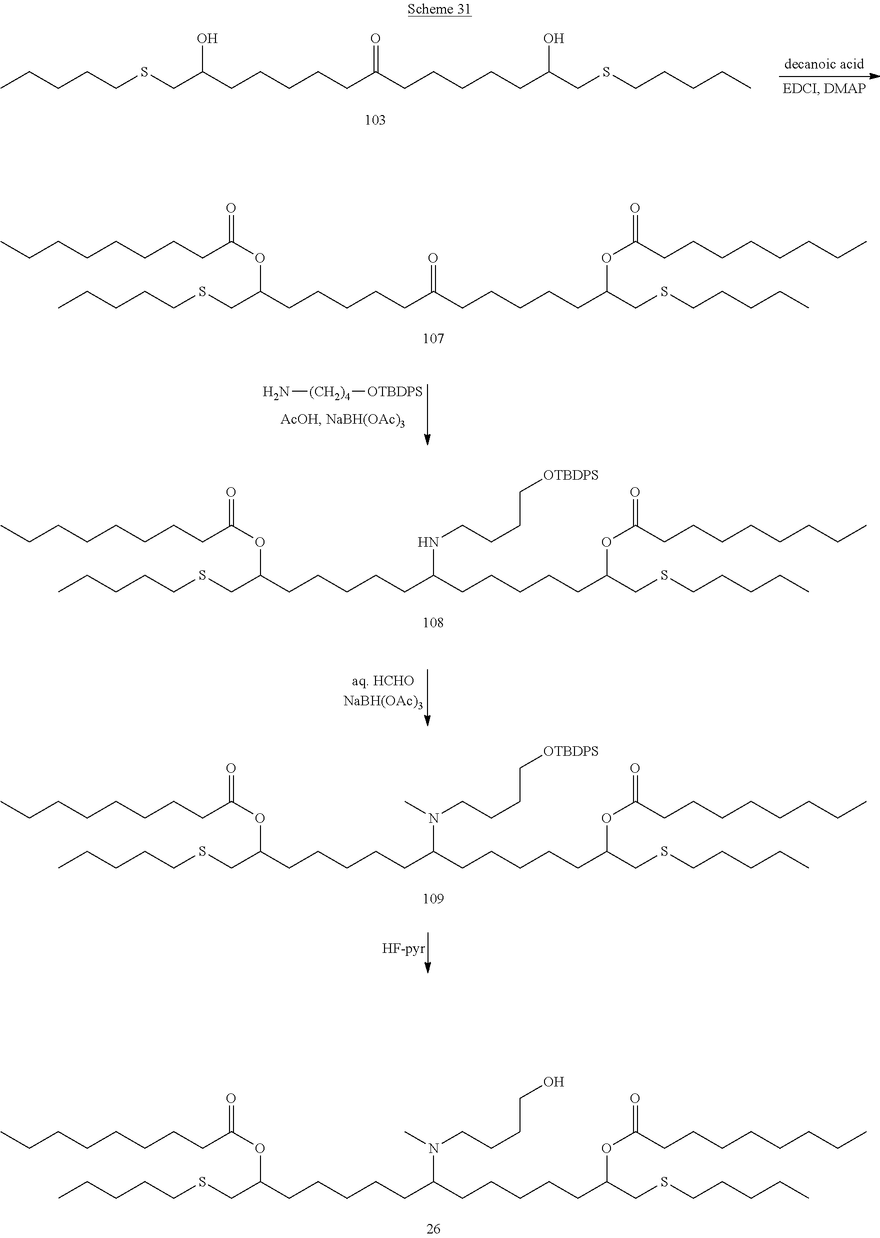

Lipid 27 can be obtained from ketone 69 of Scheme 12 by the method shown in Scheme 27 above (Scheme 32).

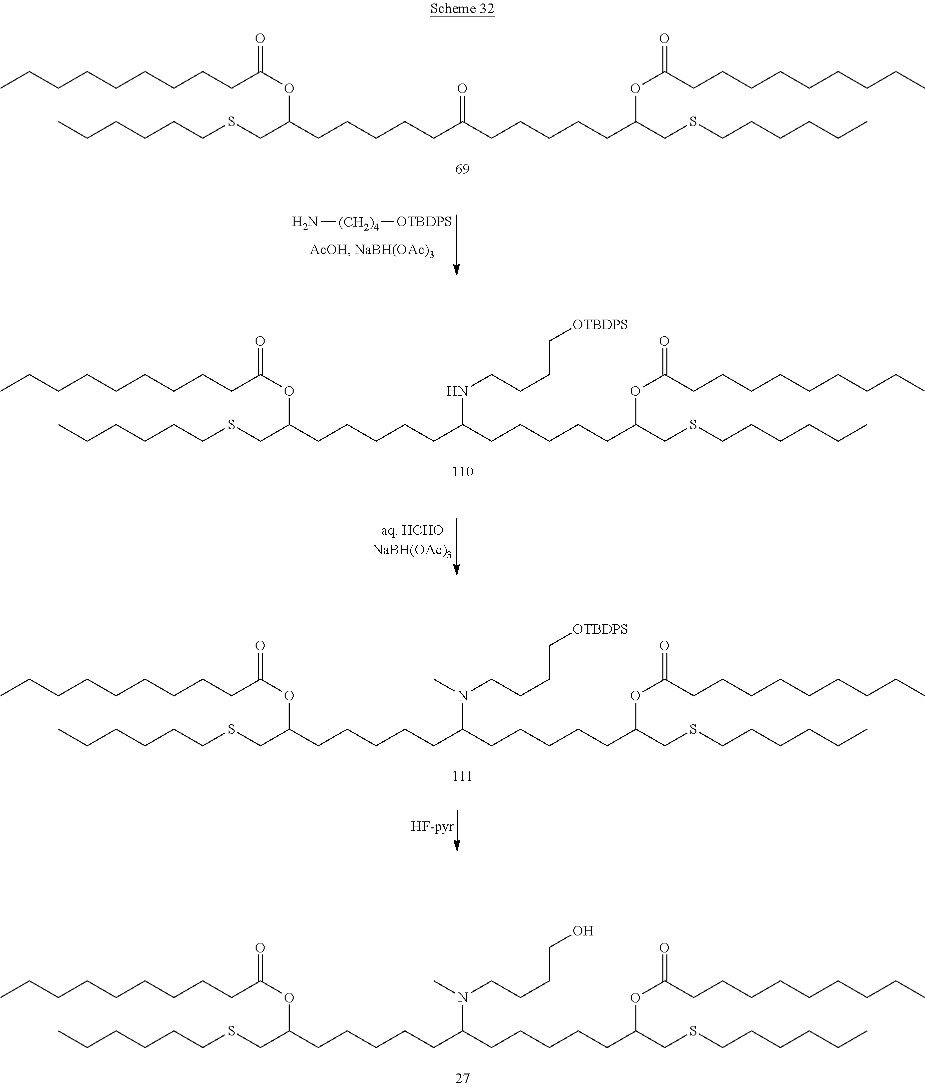

Lipid 28 can be prepared starting with the N-propylation of secondary amine 110. This can be done, for example, by reaction of 110 with propanal (propionaldehyde) in an appropriate solvent, for example, 1,2-dichloroethane, in the presence of a reducing agent such as a borohydride reagent, for example, sodium triacetoxyborohydride, sodium cyanoborohydride and the like, optionally in the presence of a catalyst such as acetic acid (Scheme 33). The resulting 112 is then transformed into lipid 28 by the same method shown in Scheme 27 above.

Lipid 29 can be prepared starting with the N-isobutylation of secondary amine 110. This can be done, for example, by reaction of 110 with 2-methylpropanal (isobutyraldehyde) in an appropriate solvent, for example, 1,2-dichloroethane, in the presence of a reducing agent like a borohydride

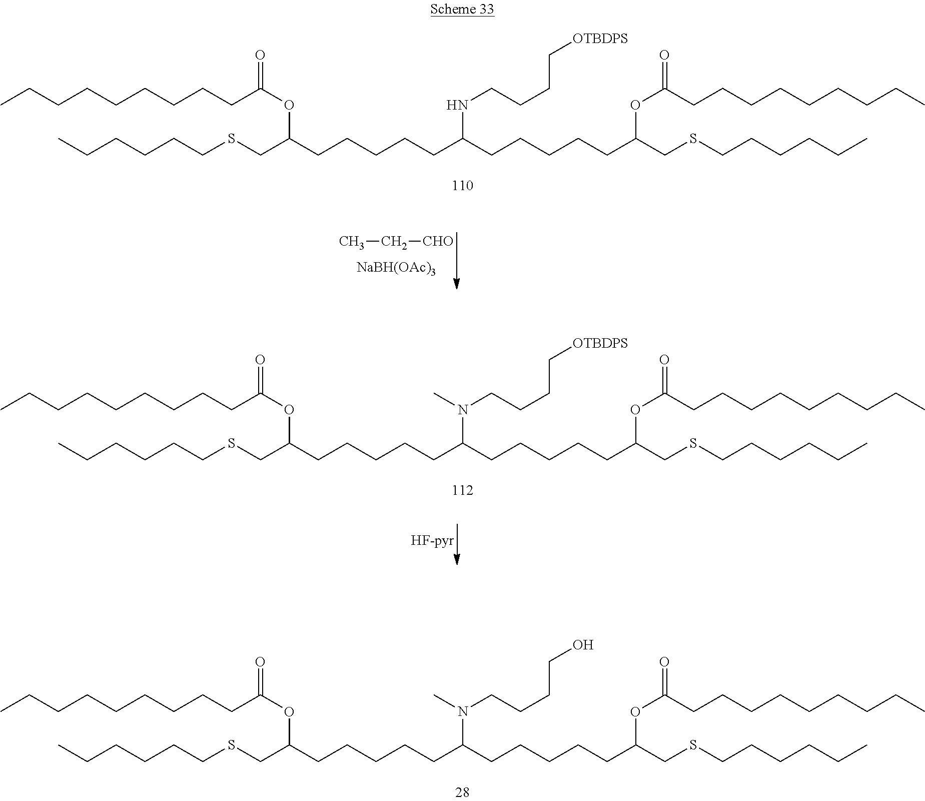

reagent, for example, sodium triacetoxyborohydride, sodium cyanoborohydride and the like, optionally in the presence of a catalyst such as acetic acid (Scheme 34). The resulting 113 is then transformed into lipid 29 by the same method shown in Scheme 27 above.

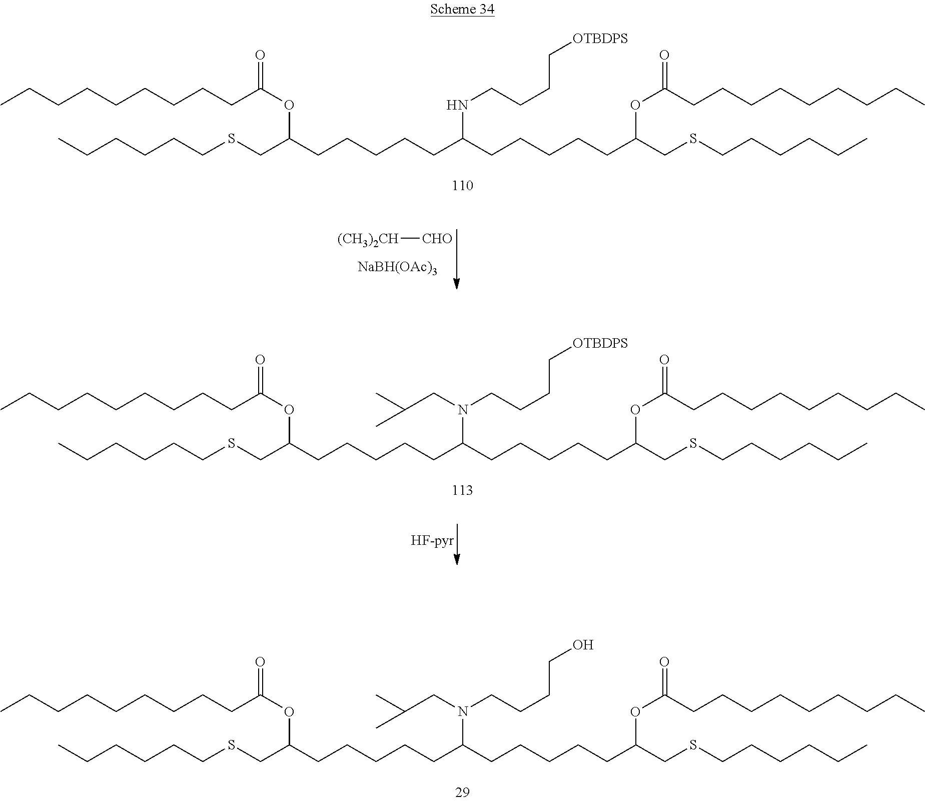

Lipid 30 can be prepared from ketone 76 of Scheme 15 by the method outlined in Scheme 27 above (Scheme 35).

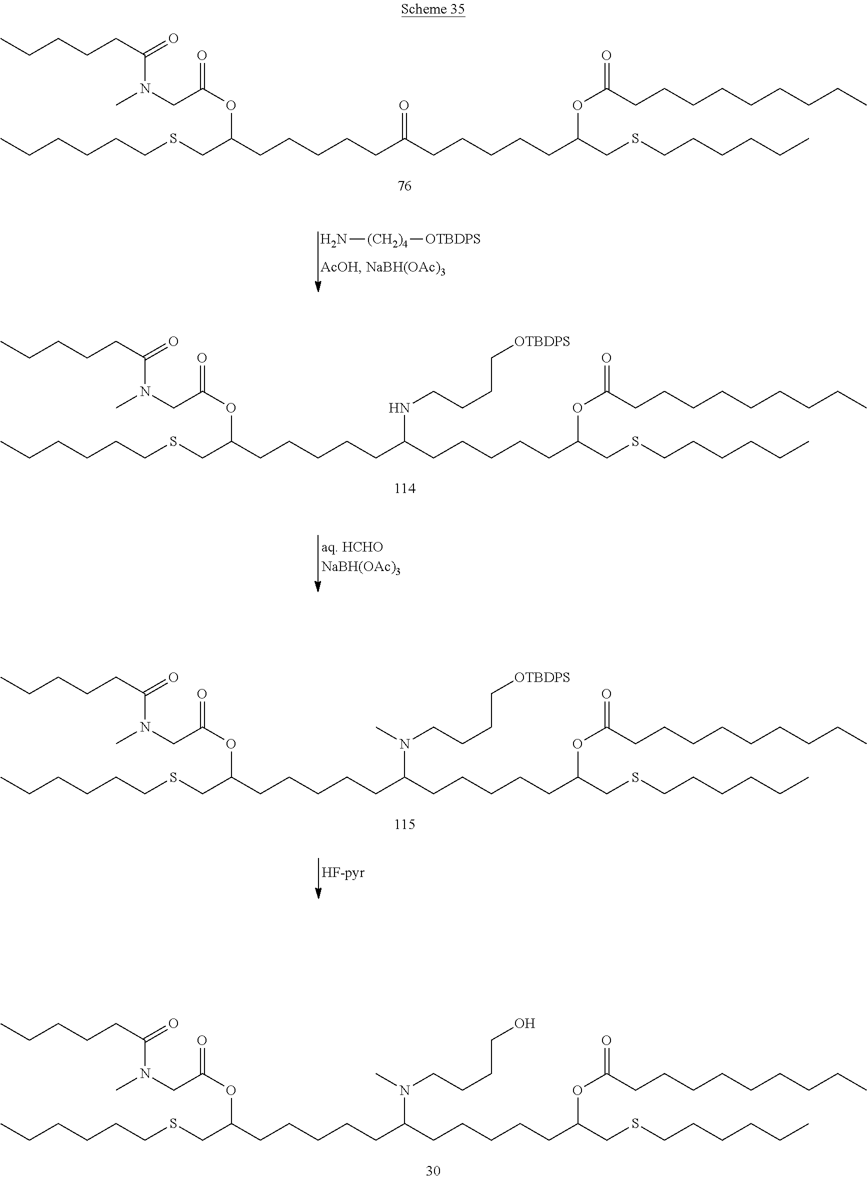

Lipid 31 can be prepared starting with the reaction of bis-epoxy ketone 67 of Scheme 12 with 1-heptanethiol, followed by esterification of the resulting 116 with nonanoic acid (Scheme 36). Ketone 117 thus obtained can then be transformed into lipid 31 by the method shown in Scheme 27 above.

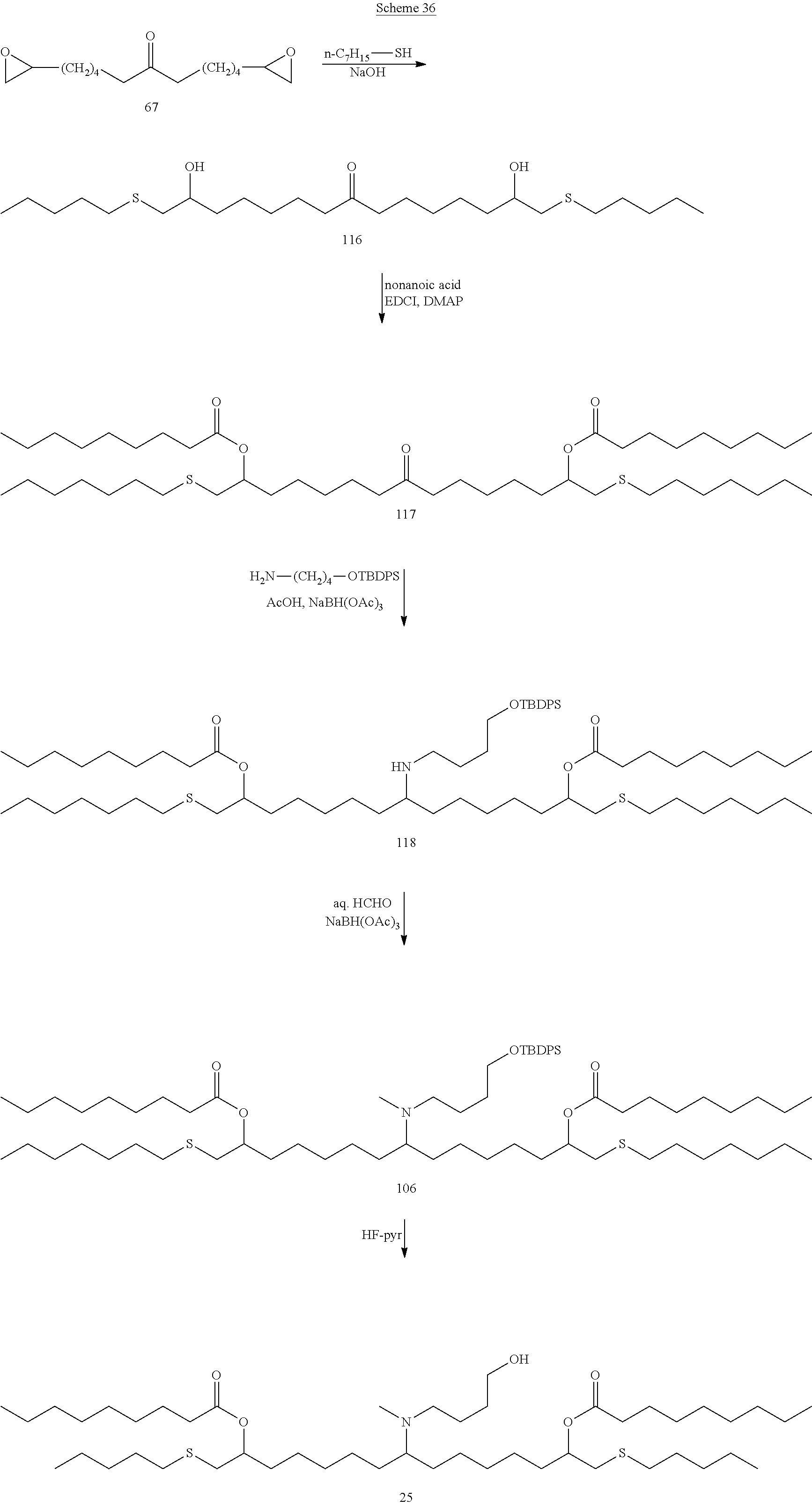

Lipid 32 can be prepared from ketone 81 of Scheme 17 by the method outlined in Scheme 27 above (Scheme 37).

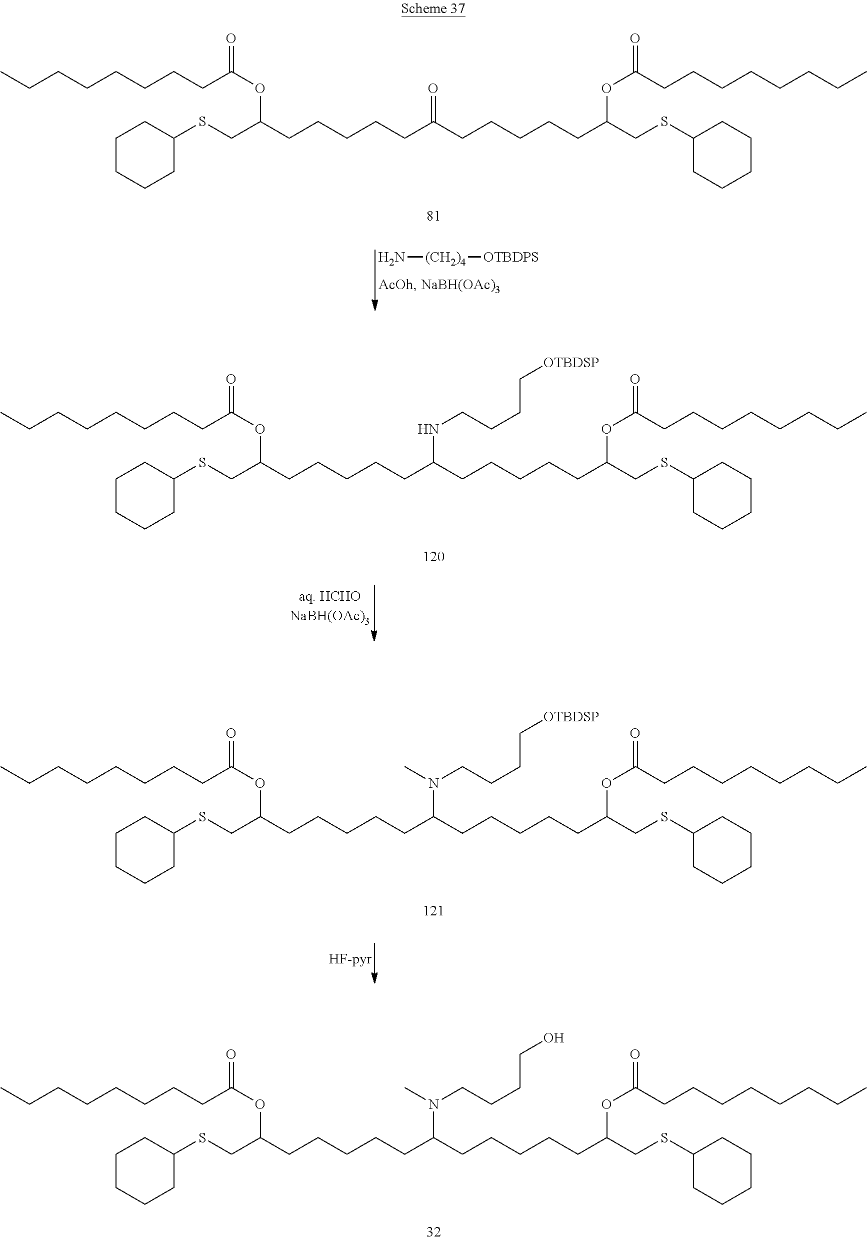

### Formulation of the Above Lipids in a Delivery Vehicle

The lipids of the disclosure may be formulated in a variety of drug delivery vehicles (also referred to herein as a “delivery vehicle”) known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, a lipid having the structure of Formula A of the disclosure is formulated in a delivery vehicle by mixing them with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, a DSPC-cholesterol conjugate or mixtures thereof. These lipids may be synthesized or obtained from natural sources, such as from egg. The DSPC-cholesterol conjugate is a lipid in which one of the acyl chains is substituted with a cholesterol moiety link to the head group by a succinate linker.

A suitable ceramide derivative is egg sphingomyelin or dihydrosphingomyelin.

Delivery vehicles incorporating the lipids of the disclosure can be prepared using a wide variety of well described formulation methodologies known to those of skill in the art, including but not limited to extrusion, ethanol injection and in-line mixing. In one embodiment, the preparation method is an in-line mixing technique in which aqueous and organic solutions are mixed using a rapid-mixing device as described in Kulkarni et al., 2018, ACS Nano, 12:4787 and Kulkarni et al., 2017, Nanoscale, 36:133347, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid. Nanoparticles comprising lipids of the disclosure may alternatively be prepared from polymers without lipids. Such nanoparticles may comprise a concentrated core of a therapeutic agent that is surrounded by a polymeric shell or may have a solid or a liquid dispersed throughout a polymer matrix.

Lipids described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

Lipids described herein may be incorporated into a micelle. Micelles are self-assembling particles composed of amphipathic lipids or polymeric components that are utilized for the delivery of agents present in the hydrophobic core.

### Delivery of Nucleic Acid, Genetic Material, Proteins, Peptides or Other Charged Agents

Lipids disclosed herein may facilitate the incorporation of a compound or molecule (referred to herein also as “cargo” or “cargo molecule”) bearing a net negative or positive charge into the delivery vehicle and subsequent delivery to a target cell in vitro or in vivo.

In one embodiment, the cargo molecule is genetic material, such as a nucleic acid. The nucleic acid includes, without limitation, RNA, including small interfering RNA (siRNA), small nuclear RNA (snRNA), micro RNA (miRNA), messenger RNA (mRNA) or DNA such as vector DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one embodiment, the cargo is an mRNA, which includes a polynucleotide that encodes at least one peptide, polypeptide or protein. The mRNA includes, but is not limited to, small activating RNA (saRNA) and trans-amplifying RNA (taRNA), as described in WO 2022/251953A1, which is incorporated herein by reference.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is a chemically synthesized molecule, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to encapsulate mRNAs of a variety of lengths. In some embodiments, the present disclosure may be used to encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on 5′ end, and a “tail” on 3′ end. The presence of the cap is important in providing resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In a further embodiment, the mRNA is circular. Advantageously, such mRNA lacks 5′ and 3′ ends and thus may be more stable in vivo due to its resistance to degradation by exonucleases. The circular mRNA may be prepared by any known method, including any one of the methods described in Deviatkin et al., 2023, “Cap-Independent Circular mRNA Translation Efficiency”, Vaccines, 11 (2), 238, which is incorporated herein by reference. Translation of the circular mRNA is carried out by a cap-independent initiation mechanism.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences may be incorporated into mRNA for in vitro studies or for in vivo studies in animal models to assess biodistribution.

In another embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

The siRNA encompassed by embodiments of the disclosure may be used to specifically inhibit expression of a wide variety of target polynucleotides. The siRNA molecules targeting specific polynucleotides may be readily prepared according to procedures known in the art. An siRNA target site may be selected and corresponding siRNAs may be chemically synthesized, created by in vitro transcription, or expressed from a vector or PCR product. A wide variety of different siRNA molecules may be used to target a specific gene or transcript. The siRNA may be double-stranded RNA, or a hybrid molecule comprising both RNA and DNA, e.g., one RNA strand and one DNA strand. The siRNA may be of a variety of lengths, such as 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA. Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic importance in diseases that are an outcome of splicing defects.

In another embodiment, the cargo is a DNA vector as described in co-owned and co-pending WO 2022/251959, which is incorporated herein by reference. The DNA vectors may be administered to a subject for the purpose of repairing, enhancing or blocking or reducing the expression of a cellular protein or peptide. Accordingly, the nucleotide polymers can be nucleotide sequences including genomic DNA, cDNA, or RNA.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will preferably have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16 (11): 1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present method can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the DNA vector is a nanoplasmid or a minicircle.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the lipids described herein. Optionally, a ribonucleoprotein complex may be incorporated into a delivery vehicle comprising the lipid described herein. Likewise, the disclosure includes embodiments in which genetic material encoding DNA binding and cleavage domains of a zinc finger nuclease or TALEN system are incorporated into a delivery vehicle together with the lipids of the disclosure.

While a variety of nucleic acid cargo molecules are described above, it will be understood that the above examples are non-limiting and the disclosure is not to be considered limiting with respect to the particular cargo molecule encapsulated in the delivery vehicle.

For example, the lipids described herein may also facilitate the incorporation of proteins and peptides into a delivery vehicle, which includes ribonucleoproteins. This includes both linear and non-linear peptides, proteins or ribonucleoproteins.

While pharmaceutical compositions are described above, the lipids described herein can be a component of any nutritional, cosmetic, cleaning or foodstuff product.

### Pharmaceutical Formulations

The ionizable lipids of the disclosure may be present in a salt form. The salt is typically a pharmaceutically acceptable salt. Salts derived from inorganic bases include aluminum, ammonium, calcium, copper, ferric, ferrous, lithium, magnesium, manganic salts, manganous, potassium, sodium, and zinc. In one embodiment, the base is selected from ammonium, calcium, magnesium, potassium and sodium. Salts derived from pharmaceutically acceptable organic non-toxic bases include salts of primary, secondary and tertiary amines, substituted amines including naturally occurring substituted amines, cyclic amines and the like.

In some embodiments, the delivery vehicle comprising the cargo molecule is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In one embodiment, the pharmaceutical compositions is administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The pharmaceutical composition comprises pharmaceutically acceptable salts and/or excipients.

The compositions described herein may be administered to a patient. The term patient as used herein includes a human or a non-human subject.

The following examples are given for the purpose of illustration only and not by way of limitation on the scope of the invention.

### EXAMPLES

### Materials

The lipid 1,2-distearoyl-sn-glycero-3-phosphorylcholine (DSPC) and 1,2-dimyristoyl-rac-glycero-3-methoxypolyethylene glycol-2000 (PEG-DMG) were purchased from Avanti Polar Lipids (Alabaster, AL). Cholesterol and 10× Phosphate Buffered Saline (pH 7.4) were purchased from Sigma Aldrich (St Louis, MO). The ionizable amino-lipid was synthesized as previously described in WO 2022/246555, which is incorporated herein by reference.

An mRNA encoding firefly luciferase purchased from APExBIO Technology LLC (Houston, TX) was used to analyse luciferase activity.

### Methods

Preparation of Lipid Nanoparticles (LNP) Containing mRNA

Lipids 1 or 2 described herein, DSPC, cholesterol, and PEG-DMG, were dissolved in ethanol at the appropriate ratios to a final concentration of 10 mM total lipid. Nucleic acid (siRNA or mRNA) was dissolved in an appropriate buffer such as 25 mM sodium acetate pH 4 or sodium citrate pH 4 to a concentration necessary to achieve the appropriate amine-to-phosphate ratios. The aqueous and organic solutions were mixed using a rapid-mixing device as described in Kulkarni et al., 2018, ACS Nano, 12:4787 and Kulkarni et al., 2017, Nanoscale, 36:133347 (each incorporated herein by reference) at a flow rate ratio of 3:1 (v/v; respectively) and a total flow rate of 20 mL/min. The resultant mixture was dialyzed directly against 1000-fold volume of PBS pH 7.4. All formulations were concentrated using an Amicon™ centrifugal filter unit and analysed using the methods described below.

### Analysis of LNP

Particle size analysis of LNPs in PBS was carried out using backscatter measurements of dynamic light scattering with a Malvern Zetasizer™ (Worcestershire, UK). The reported particle sizes correspond to the number-weighted average diameters (nm). Total lipid concentrations were determined by extrapolation from the cholesterol content, which was measured using the Cholesterol E-Total Cholesterol Assay (Wako Diagnostics, Richmond, VA) as per the manufacturer's recommendations. Encapsulation efficiency of the formulations was determined using the Quant-iT RiboGreen™ Assay kit (Invitrogen, Waltham, MA). Briefly, the total siRNA or mRNA content in solution was measured by lysing lipid nanoparticles in a solution of TE containing 2% Triton Tx-100, and free DNA vector in solution (external to LNP) was measured based on the RiboGreen™ fluorescence in a TE solution without Triton. Total siRNA or mRNA content in the formulation was determined using a modified Bligh-Dyer extraction procedure. Briefly, LNP formulations containing siRNA or mRNA were dissolved in a mixture of chloroform, methanol, and PBS that results in a single phase and the absorbance at 260 nm measured using a spectrophotometer.

### In Vivo Analysis in CD-1 Mice

LNP-mRNA encoding firefly luciferase were injected intravenously (tail-vein) into 6-8 week old CD-1 mice. Four hours following injection, the animals were euthanized and the liver and spleen and isolated. Tissue was homogenized in Glo Lysis buffer and a luciferase assay performed using the Steady Glo Luciferase assay kit (as per manufacturers recommendations).

### Organic Synthesis of Lipids 5-32.

Unless otherwise specified, reagents and solvents were commercial products and were used without purification, except THF (freshly distilled from Na/benzophenone under Ar), CH₂Cl₂ (freshly distilled from CaH₂ under Ar). “Dry methanol” was freshly distilled from magnesium turnings. All reactions were performed under inert atmosphere (nitrogen or argon). Reaction mixture from aqueous workups were dried by passing over a plug of anhydrous Na₂SO₄ held in a filter tube and concentrated under reduced pressure on a rotary evaporator. Thin-layer chromatography was performed on silica gel plates coated with silica gel (Merck 60 F254 plates). Visualization of the developed chromatogram was performed by staining with I₂ or potassium permanganate solution. Chromatographic purifications were performed on a Biotage ISCO system. ¹H and ¹³C nuclear magnetic resonance (NMR) spectra were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the δ scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

The synthesis of lipid 5 from 4-amino-1-butanol was carried out as set forth below. As discussed, the synthesis of lipids of the type 5 involves subjecting 4-amino-1-butanol to mono-N-alkylation or di-N-alkylation with certain alkyl halides or sulfonates under appropriate conditions. This technology is as set forth in co-owned and co-pending WO 2023/173203 (incorporated herein by reference).

The synthesis of lipids 5-32 was carried out as set forth below. As discussed, the synthesis of said lipids involves subjecting certain esters or lactones to Claisen condensation under Mukaiyama conditions. This technology is as set forth in co-owned and co-pending WO 2022/246555 (incorporated herein by reference). The products of such Claisen reactions are subsequently converted into the final products as outlined in the Schemes above and as described below.

### Example 1: Methods for Chemically Synthesizing Ionizable Lipids

### (A) Preparation of Building Blocks

(i) N-(4-((tert-Butyldiphenylsilyl)oxy)butyl)-N-(hept-6-en-1-yl)hept-6-en-1-amine

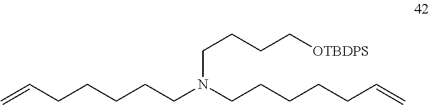

(42). A mixture of O-TBDPS protected 4-amino-1-butanol 40 (841 mg, 2.57 mmol), 7-bromohept-1-ene (1.00 g, 5.65 mmol), and K₂CO₃ (859 mg, 6.22 mmol) in MeCN (15.0 mL) was stirred at 80° C. in a sealed reaction vessel for 18 hours. The mixture was cooled, diluted with water (15 mL) and extracted with CH₂Cl₂ (3×15 mL). The combined organics were dried (Na₂SO₄) and concentrated. The residue was purified by silica chromatography (0-5% MeOH in DCM) to yield amine 42 (802 mg, 60%) as an oil. ¹H NMR (400 MHz, CDCl₃) δ 7.72-7.57 (m, 4H), 7.48-7.31 (m, 6H), 5.89-5.69 (m, 2H), 5.06-4.86 (m, 4H), 3.78-3.63 (m, 2H), 3.01-2.28 (m, 6H), 2.04 (q, J=7.1 Hz, OH), 1.60-1.21 (m, 20H), 1.04 (s, 9H).

(ii) Trideca-1,12-dien-7-one (61). To a solution of methyl hept-6-enoate (5.95 g, 41.9

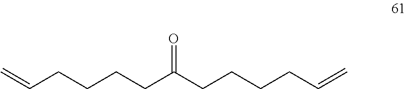

mmol) and NBu₃ (18.0 mL, 75.4 mmol) in toluene (80.0 mL) was added dropwise a solution of TiCl₄ (6.89 mL, 62.9 mmol) in toluene (40.0 mL) at 0° C. under a nitrogen atmosphere. The reaction was warmed to room temperature and stirred for 2 hours. Water (40 mL) was added at 0° C. The biphasic mixture was extracted with toluene (2×40 mL). The combined organics were concentrated, the residue was dissolved in a EtOH (70 mL) and 25% NaOH (25 mL) was added. The mixture was stirred for 2 hours, concentrated to 25% volume, acidified to pH 2 with conc. HCl, and extracted with 50:50 Hexanes/Et₂O (3×40 mL). The combined organics were washed with brine, dried (Na₂SO₄) and concentrated. The residue was purified by silica chromatography (0-8% EtOAc in Hexanes) to yield ketone 61 as an oil (3.7 g, 91%). ¹H NMR (400 MHz, CDCl₃) δ 5.79 (ddt, J=16.9, 10.2, 6.7 Hz, 2H), 5.07-4.86 (m, 4H), 2.39 (t, J=7.4 Hz, 4H), 2.20-1.98 (m, 4H), 1.71-1.49 (m, 4H), 1.49-1.33 (m, 4H).

(iii) Pentadeca-1,14-dien-8-one (66). Prepared from methyl oct-7-enoate by the

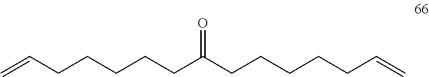 [SMILES: C=CCCCCCC(=O)CCCCCC=C]

procedure of part (ii) above. ¹H NMR (400 MHz, CDCl₃) δ 5.79 (ddt, J=16.9, 10.2, 6.6 Hz, 2H), 5.07-4.84 (m, 4H), 2.38 (t, J=7.4 Hz, 4H), 2.10-1.99 (m, 4H), 1.62-1.52 (m, 4H), 1.45-1.34 (m, 4H), 1.34-1.23 (m, 4H).

(iv) 1,9-di(Oxiran-2-yl)nonan-5-one (62). Solid mCPBA (13.9 g, ˜50% pure) was

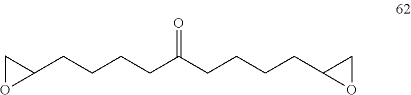 [SMILES: O=C(CCCCC1CO1)CCCCC1CO1]

added to a solution of ketone 61 (3.9 g, 20.0 mmol) in DCM (70.0 mL) at 0° C. The mixture was warmed to room temperature and stirred for 2 hours. The mixture was cooled to 0° C. and quenched with sat. aq. sodium sulfite and diluted with water (20 mL). The layers were separated, and the organics were washed with 1 N NaOH (3×30 mL), dried (Na₂SO₄) and concentrated to yield the bis-epoxide 62 (4.1 g, 90%). ¹H NMR (400 MHz, CDCl₃) δ 2.94-2.87 (m, 2H), 2.74 (dd, J=5.0, 3.9 Hz, 2H), 2.46 (dd, J=5.0, 2.7 Hz, 2H), 2.41 (t, J=7.3 Hz, 4H), 1.73-1.34 (m, 12H).

(v). 1,11-di(Oxiran-2-yl)undecan-6-one (67). Prepared from ketone 66 by procedure

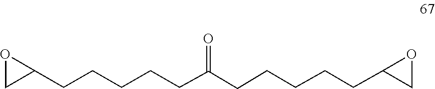 [SMILES: O=C(CCCCCC1CO1)CCCCCC1CO1]

(iv) above. ¹H NMR (400 MHz, CDCl₃) δ 2.94-2.83 (m, 2H), 2.76-2.69 (m, 2H), 2.44 (dd, J=5.0, 2.7 Hz, 2H), 2.38 (td, J=7.5, 1.6 Hz, 4H), 1.65-1.20 (m, 16H).

(vi) N-(4-((tert-Butyldiphenylsilyl)oxy)butyl)-5-(oxiran-2-yl)-N-(5-(oxiran-2-yl)pentyl)-pentan-1-amine oxide (43). Solid mCPBA (1.59 g, ˜50% pure) was added to a

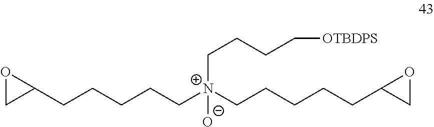

solution of 42 (800 mg, 1.54 mmol) in DCM (10.0 mL) at 0° C. The mixture was warmed to room temperature and stirred for 3 hours. The mixture was 43 cooled to 0° C. and quenched with sat. aq. sodium sulfite and diluted with water (10.0 mL). The layers were separated, and the organics were washed with 1 N NaOH (3×30.0 mL), dried (Na₂SO₄) and concentrated to yield the bis-epoxide 43 as a waxy white solid (629 mg, 72%) which was used in the next step without further purification. ¹H NMR (400 MHz, CDCl₃) δ 7.71-7.60 (m, 4H), 7.51-7.34 (m, 6H), 3.71 (t, J=5.9 Hz, 2H), 3.32-3.12 (m, 6H), 2.92-2.84 (m, 2H), 2.78-2.70 (m, 2H), 2.49-2.41 (m, 2H), 2.01-1.34 (m, 20H), 1.04 (s, 9H).

(vii) General procedure for epoxide opening with a thiol. To a well-stirred solution of a bis-epoxide (1 mmol) and a thiol (2.2 mmol, 2.2 equiv) in EtOH (10 mL) maintained under inert atmosphere was added solid NaOH (4 equiv). The mixture was heated at reflux for 2 hours, cooled, diluted with water (20 mL) and extracted with DCM (3×15.0 mL). The combined organics were dried (NaSO₄) and concentrated. The residue was purified by silica chromatography (0-50% EtOAc in hexanes) to yield the desired product (75-80%). The following compounds were thus prepared:

(viii) N-(4-((tert-butyldiphenylsilyl)oxy)butyl)-6-hydroxy-N-(6-hydroxy-7-(pentylthio)hept-yl)-7-(pentylthio)heptan-1-amine oxide (44) and 9-(6-hydroxy-7-(pentylthio)heptyl)-2,2-dimethyl-3,3-diphenyl-4-oxa-17-thia-9-aza-3-siladocosan-15-ol (45). Compound 44 was obtained from 43 and 1-pentanethiol by the general procedure of part (vii). ¹H NMR (400 MHz, CDCl₃) δ 3.69-3.55 (m, 2H), 2.73 (dd, J=13.6, 3.3 Hz, 2H), 2.51 (t, J=7.4 Hz, 4H), 2.46-2.34 (m, 6H), 1.67-1.23 (m, 28H), 0.90 (t, J=7.0 Hz, 6H). A solution of amine oxide 44 (510 mg, 0.657 mmol) and triphenylphosphine (517 mg, 1.97 mmol) in glacial HOAc

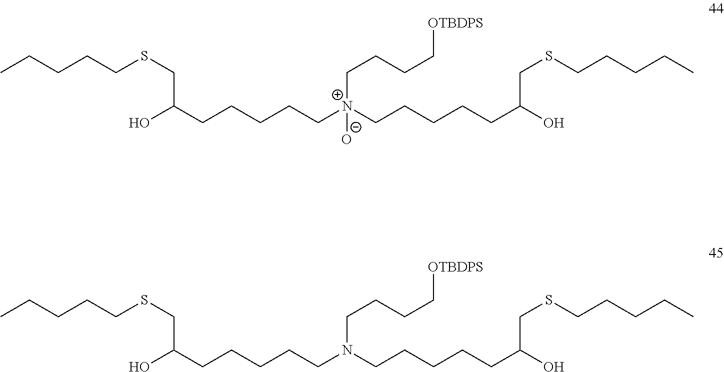

(8.00 mL) was heated to reflux for 4 hours. The mixture was then diluted with DCM (10.0 mL) and washed with water (3×15.0 mL), brine (15.0 mL), dried (Na₂SO₄) and concentrated. The residue was purified by silica chromatography (0-40% MeOH in DCM) to yield the amine 45 (255 mg, 51%). ¹H NMR (400 MHz, CDCl₃) δ 7.69-7.62 (m, 4H), 7.47-7.33 (m, 6H), 3.67 (t, J=5.9 Hz, 2H), 3.65-3.58 (m, 2H), 2.71 (dd, J=13.6, 3.5 Hz, 2H), 2.66-2.56 (m, 6H), 2.51 (t, J=7.5 Hz, 4H), 2.43 (dd, J=13.6, 8.9 Hz, 2H), 1.66-1.26 (m, 32H), 1.04 (s, 9H), 0.89 (t, J=7.1 Hz, 6H).

(ix) 1,13-bis(Cyclohexylthio)-2,12-dihydroxytridecan-7-one (63). Prepared from 62

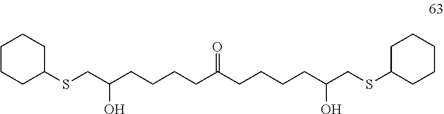 [SMILES: O=C(CCCCC(O)CSC1CCCCC1)CCCCC(O)CSC1CCCCC1]

and cyclohexanethiol by the procedure of part (vii). ¹H NMR (400 MHz, CDCl₃) δ 3.66-3.56 (m, 2H), 2.78 (dd, J=13.5, 3.4 Hz, 2H), 2.69-2.57 (m, 2H), 2.49-2.37 (m, 6H), 2.03-1.89 (m, 6H), 1.81-1.71 (m, 4H), 1.67-1.20 (m, 22H).

(x) 1,15-bis(Hexylthio)-2,14-dihydroxypentadecan-8-one (68). Prepared from 67 and 1-hexanethiol by the procedure of part (vii). ¹H NMR (400 MHz, CDCl₃) δ 3.69-3.55 (m, 2H),

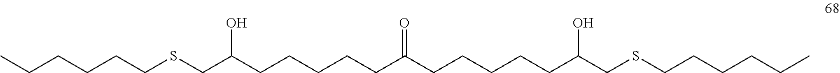 [SMILES: CCCCCCSCC(O)CCCCCC(=O)CCCCCC(O)CSCCCCCC]

### 2.73 (dd, J=13.6, 3.3 Hz, 2H), 2.51 (t, J=7.4 Hz, 4H), 2.46-2.34 (m, 6H), 1.67-1.23 (m, 32H), 0.90 (t, J=7.0 Hz, 6H).

(xi) 1,15-bis(cyclohexylthio)-2,14-dihydroxypentadecan-8-one (78). Prepared from

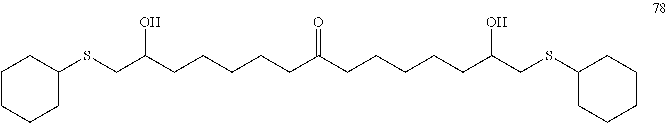 [SMILES: O=C(CCCCCC(O)CSC1CCCCC1)CCCCCC(O)CSC1CCCCC1]

67 and cyclohexanethiol by the procedure of part (vii). ¹H NMR (400 MHz, CDCl₃) δ 3.65-3.53 (m, 2H), 2.83-2.73 (m, 2H), 2.70-2.57 (m, 2H), 2.55-2.30 (m, 6H), 2.07-1.85 (m, 4H), 1.82-1.70 (m, 4H), 1.58 (tdt, J=14.8, 7.3, 3.9 Hz, 6H), 1.51-1.39 (m, 6H), 1.39-1.17 (m, 16H).

(xii) 2,12-Dihydroxy-1,13-bis(pentylthio)tridecan-7-one (83). Prepared from 62 and

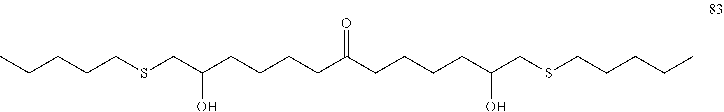 [SMILES: CCCCCSCC(O)CCCCC(=O)CCCCC(O)CSCCCCC]

1-pentanethiol by the procedure of part (vii). ¹H NMR (400 MHz, CDCl₃) δ 3.68-3.55 (m, 2H), 2.79-2.68 (m, 2H), 2.58-2.36 (m, 10H), 1.66-1.52 (m, 8H), 1.53-1.43 (m, 6H), 1.41-1.25 (m, 10H), 0.95-0.85 (m, 6H).

(xiii) 1,13-bis(Heptylthio)-2,12-dihydroxytridecan-7-one (96). Prepared from 62 and

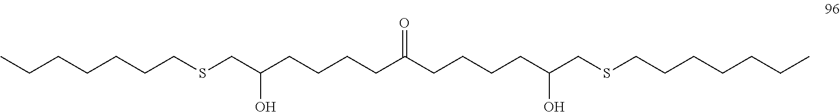 [SMILES: CCCCCCCSCC(O)CCCCC(=O)CCCCC(O)CSCCCCCCC]

1-heptanethiol by the procedure of part (vii). ¹H NMR (400 MHz, CDCl₃) δ 3.70-3.56 (m, 2H), 2.72 (dd, J=13.6, 3.3 Hz, 2H), 2.58-2.34 (m, 10H), 1.70-1.53 (m, 8H), 1.53-1.42 (m, 6H), 1.42-1.22 (m, 18H), 0.92-0.83 (m, 6H).

(xiv) 2,14-Dihydroxy-1,15-bis(pentylthio)pentadecan-8-one (103). Prepared from 67

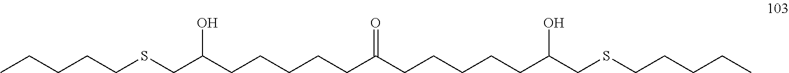 [SMILES: CCCCCSCC(O)CCCCCC(=O)CCCCCC(O)CSCCCCC]

and 1-pentanethiol by the procedure of part (vii). ¹H NMR (400 MHz, CDCl₃) δ 3.69-3.55 (m, 2H), 2.73 (dd, J=13.6, 3.3 Hz, 2H), 2.51 (t, J=7.4 Hz, 4H), 2.46-2.34 (m, 6H), 1.67-1.23 (m, 28H), 0.90 (t, J=7.0 Hz, 6H).

(xv) 1,15-bis(Heptylthio)-2,14-dihydroxypentadecan-8-one (116). Prepared from 67

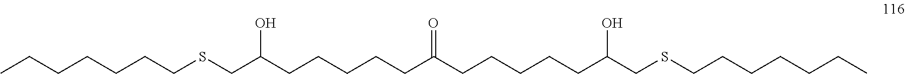 [SMILES: CCCCCCCSCC(O)CCCCCC(=O)CCCCCC(O)CSCCCCCCC]

and 1-heptanethiol by part (vii). ¹H NMR (400 MHz, CDCl₃) δ 3.69-3.55 (m, 2H), 2.73 (dd, J=13.6, 3.3 Hz, 2H), 2.51 (t, J=7.4 Hz, 4H), 2.46-2.34 (m, 6H), 1.67-1.23 (m, 36H), 0.90 (t, J=7.0 Hz, 6H).

### (B) Preparation of Symmetrical Diester Derivatives of Dihydroxy Compounds of Part (A).

(i) General procedure. A solution of a dihydroxy compound (1 mmol, 1 equiv), a carboxylic acid (2.4 mmol, 1.2 equiv), EDCI*HCl (2.5 mmol, 1.25 equiv), and DMAP (2.4 mmol) in DCM (4 mL) was stirred at rt under a nitrogen atmosphere 18 hours. The reaction was concentrated, and the residue purified by silica chromatography (0-10% EtOAc in Hexanes) to yield the diester (80-90%) as an oil. The following compounds were thus obtained:

(ii) 9-(6-(Decanoyloxy)-7-(pentylthio)heptyl)-2,2-dimethyl-3,3-diphenyl-4-oxa-17-thia-9-aza-3-siladocosan-15-yl decanoate (46). Obtained from 45 and decanoic acid. ¹H NMR (400 MHz, CDCl₃) δ 7.74-7.62 (m, 4H), 7.48-7.33 (m, 6H), 4.99-4.89 (m, 2H), 3.72-3.62 (m,

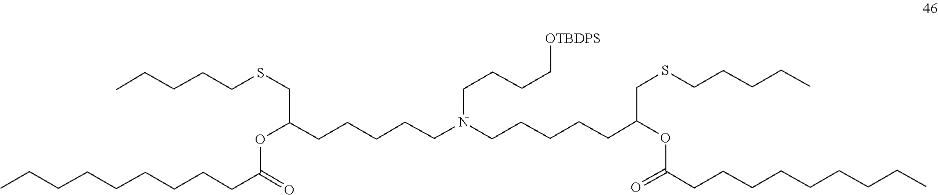

### 2H), 2.69-2.57 (m, 4H), 2.53 (td, J=7.3, 1.8 Hz, 4H), 2.45-2.24 (m, 10H), 1.77-1.15 (m, 60H), 1.04 (s, 9H), 0.94-0.82 (m, 12H).

(iii) 1,13-bis(Cyclohexylthio)-7-oxotridecane-2,12-diyl bis(decanoate) (64). Obtained from 63 and decanoic acid. H NMR (400 MHz, CDCl₃) δ 4.98-4.81 (m, 2H), 2.77-2.57 (m, 6H),

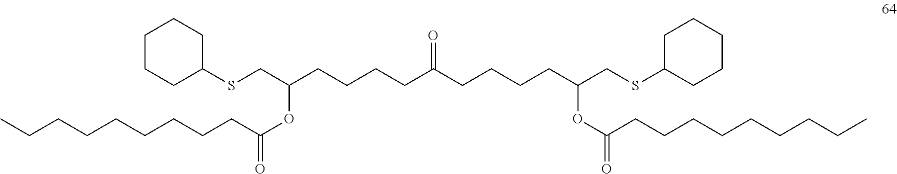 [SMILES: CCCCCCCCCC(=O)OC(CCCCC(=O)CCCCC(CSC1CCCCC1)OC(=O)CCCCCCCCC)CSC1CCCCC1]

### 2.38 (t, J=7.4 Hz, 4H), 2.29 (t, J=7.5 Hz, 4H), 2.00-1.91 (m, 4H), 1.81-1.18 (m, 56H), 0.95-0.81 (m, 6H).

(iv) 1,15-bis(hexylthio)-8-oxopentadecane-2,14-diyl bis(decanoate) (69). Obtained

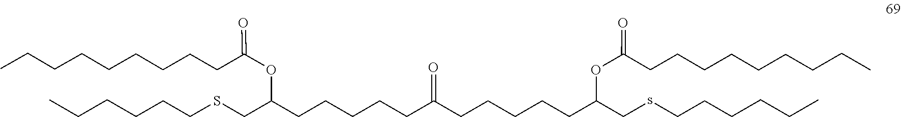

from 68 and decanoic acid. ¹H NMR (400 MHz, CDCl₃) δ 4.94 (dtd, J=8.1, 6.1, 4.4 Hz, 2H), 2.69-2.58 (m, 4H), 2.53 (td, J=7.3, 1.3 Hz, 4H), 2.37 (t, J=7.4 Hz, 4H), 2.29 (t, J=7.5 Hz, 4H), 1.78-1.47 (m, 16H), 1.46-1.20 (m, 44H), 0.93-0.81 (m, 12H).

(v) 1,15-bis(Hexylthio)-8-oxopentadecane-2,14-diyl bis(3-cyclohexylpropanoate)

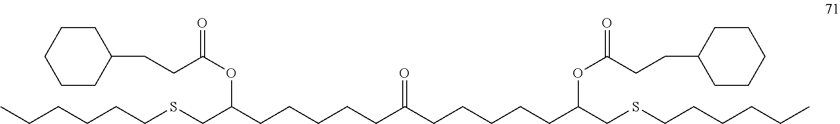 [SMILES: CCCCCCSCC(CCCCCC(=O)CCCCCC(CSCCCCCC)OC(=O)CCC1CCCCC1)OC(=O)CCC1CCCCC1]

(71). Obtained from 68 and 3-cyclohexylpropan-oic acid. ¹H NMR (400 MHz, CDCl₃) δ 5.00-4.88 (m, 2H), 2.69-2.58 (m, 4H), 2.53 (td, J=7.2, 1.4 Hz, 4H), 2.37 (t, J=7.4 Hz, 4H), 2.33-2.28 (m, 4H), 1.79-1.06 (m, 54H), 0.95-0.83 (m, 10H).

(vi) 1,15-bis(cyclohexylthio)-8-oxopentadecane-2,14-diyl bis(decanoate) (79). Obtained from 78 and decanoic acid. ¹H NMR (400 MHz, CDCl₃) δ 4.91 (dtd, J=8.1, 6.2, 4.3

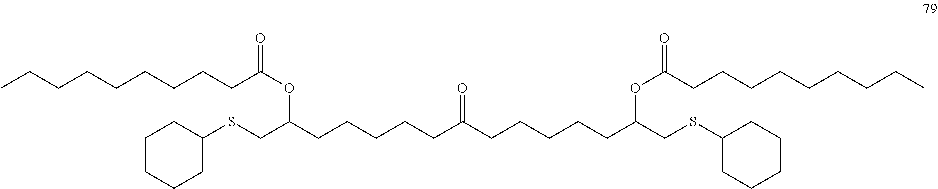 [SMILES: CCCCCCCCCC(=O)OC(CCCCCC(=O)CCCCCC(CSC1CCCCC1)OC(=O)CCCCCCCCC)CSC1CCCCC1]

### Hz, 2H), 2.73-2.58 (m, 6H), 2.36 (t, J=7.4 Hz, 6H), 2.28 (t, J=7.5 Hz, 4H), 1.99-1.89 (m, 4H), 1.81-1.48 (m, 16H), 1.37-1.19 (m, 42H), 0.92-0.82 (m, 6H).

(vii) 1,15-bis(cyclohexylthio)-8-oxopentadecane-2,14-diyl dinonanoate (81). Obtained from 78 and nonanoic acid. ¹H NMR (400 MHz, CDCl₃) δ 4.91 (dtd, J=8.2, 6.2, 4.3

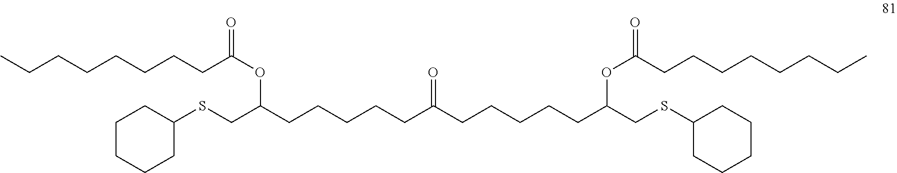 [SMILES: CCCCCCCCC(=O)OC(CCCCCC(=O)CCCCCC(CSC1CCCCC1)OC(=O)CCCCCCCC)CSC1CCCCC1]

### Hz, 2H), 2.76-2.58 (m, 6H), 2.37 (t, J=7.4 Hz, 4H), 2.29 (t, J=7.5 Hz, 4H), 1.96 (dd, J=8.9, 5.2 Hz, 4H), 1.80-1.48 (m, 18H), 1.37-1.20 (m, 38H), 0.91-0.83 (m, 6H).

(viii) 7-oxo-1,13-bis(pentylthio)tridecane-2,12-diyl bis(decanoate) (84). Obtained from 83 and decanoic acid. H NMR (400 MHz, CDCl₃) δ 5.00-4.87 (m, 2H), 2.70-2.57 (m, 4H),

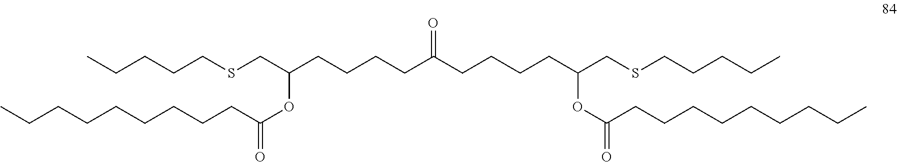 [SMILES: CCCCCCCCCC(=O)OC(CCCCC(=O)CCCCC(CSCCCCC)OC(=O)CCCCCCCCC)CSCCCCC]

### 2.53 (td, J=7.3, 1.5 Hz, 4H), 2.38 (t, J=7.4 Hz, 4H), 2.34-2.26 (m, 4H), 1.78-1.47 (m, 18H), 1.38-1.22 (m, 34H), 0.95-0.84 (m, 12H).

(ix) 1,13-bis(Heptylthio)-7-oxotridecane-2,12-diyl bis(decanoate) (97). Obtained from 96 and decanoic acid. H NMR (400 MHz, CDCl₃) δ 5.08-4.85 (m, 2H), 2.69-2.57 (m, 4H),

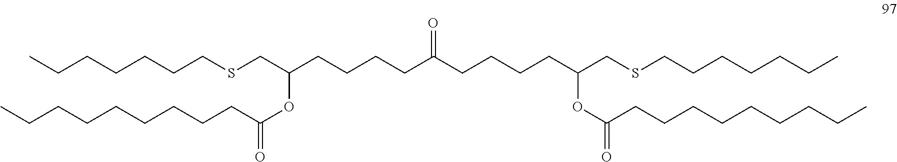 [SMILES: CCCCCCCCCC(=O)OC(CCCCC(=O)CCCCC(CSCCCCCCC)OC(=O)CCCCCCCCC)CSCCCCCCC]

### 2.53 (td, J=7.3, 1.4 Hz, 4H), 2.38 (t, J=7.4 Hz, 4H), 2.30 (t, J=7.5 Hz, 4H), 1.79-1.47 (m, 18H), 1.42-1.20 (m, 38H), 0.88 (t, J=6.8 Hz, 12H).

(x) 1,13-bis(heptylthio)-7-oxotridecane-2,12-diyl dinonanoate (100). Obtained from

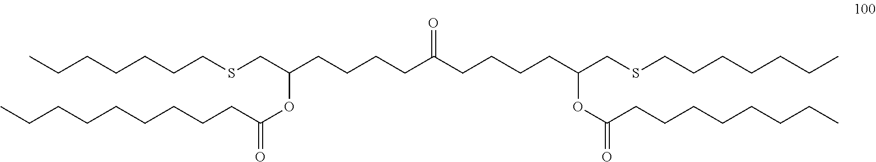 [SMILES: CCCCCCCCCC(=O)OC(CCCCC(=O)CCCCC(CSCCCCCCC)OC(=O)CCCCCCCC)CSCCCCCCC]

96 and nonanoic acid. ¹H NMR (400 MHz, CDCl₃) δ 5.08-4.85 (m, 2H), 2.69-2.57 (m, 4H), 2.53 (td, J=7.3, 1.4 Hz, 4H), 2.38 (t, J=7.4 Hz, 4H), 2.30 (t, J=7.5 Hz, 4H), 1.79-1.47 (m, 18H), 1.42-1.20 (m, 38H), 0.88 (t, J=6.8 Hz, 12H).

(xi) 8-oxo-1,15-bis(pentylthio)pentadecane-2,14-diyl dinonanoate (104). Obtained from 103 and nonanoic acid. ¹H NMR (400 MHz, CDCl₃) δ 4.94 (dtd, J=8.1, 6.1, 4.3 Hz, 2H),

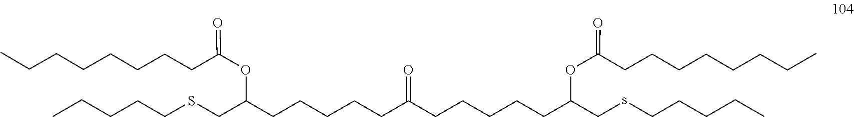

### 2.70-2.58 (m, 4H), 2.59-2.47 (m, 4H), 2.37 (t, J=7.4 Hz, 4H), 2.30 (t, J=7.5 Hz, 4H), 1.80-1.48 (m, 16H), 1.46-1.20 (m, 36H), 0.98-0.78 (m, 12H).

(xii) 8-oxo-1,15-bis(pentylthio)pentadecane-2,14-diyl bis(decanoate) (107). Obtained from 103 and decanoic acid. H NMR (400 MHz, CDCl₃) δ 5.05-4.85 (m, 2H), 2.71-2.58 (m,

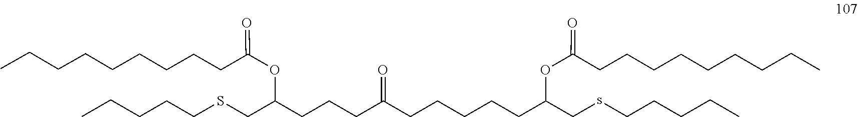

### 4H), 2.53 (td, J=7.3, 1.5 Hz, 4H), 2.37 (t, J=7.4 Hz, 4H), 2.30 (t, J=7.5 Hz, 4H), 1.78-1.49 (m, 16H), 1.39-1.21 (m, 40H), 0.96-0.83 (m, 12H).

(xiii) 1,15-bis(heptylthio)-8-oxopentadecane-2,14-diyl dinonanoate (117). Obtained from 116 and nonanoic acid H NMR (400 MHz, CDCl₃) δ 5.01-4.87 (m, 2H), 2.70-2.57 (m,

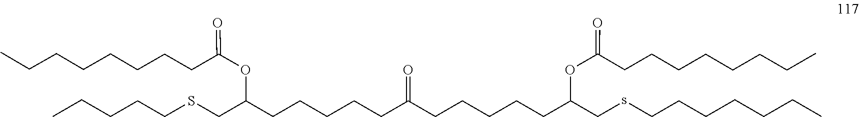

### 4H), 2.53 (td, J=7.3, 1.4 Hz, 4H), 2.37 (t, J=7.4 Hz, 4H), 2.30 (t, J=7.5 Hz, 4H), 1.76-1.49 (m, 16H), 1.42-1.18 (m, 44H), 0.92-0.84 (m, 12H).

### (C) Preparation of Unsymmetrical Diester Derivatives of Dihydroxy Compounds of Part (A).

(i) Exemplary procedure for the mono-esterification of a dihydroxy compound: 1,15-bis(hexylthio)-14-hydroxy-8-oxopentadecan-2-yl decanoate (75). A solution of 68 (0.15 g, 0.306 mmol), decanoic acid (0.0526 g, 0.306 mmol), EDCI·HCl (0.0879 g, 0.458 mmol), and DMAP (0.0261 g, 0.214 mmol) in CH₂Cl₂ (15 mL) was stirred for 16 hours at under inert

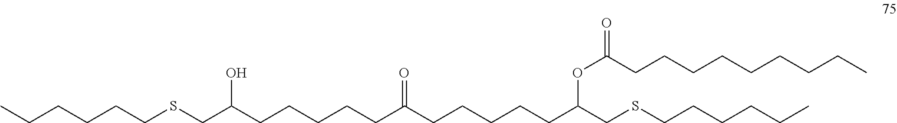 [SMILES: CCCCCCCCCC(=O)OC(CCCCCC(=O)CCCCCC(O)CSCCCCCC)CSCCCCCC]

atmosphere, then it was quenched with water and extracted with CH₂Cl₂ (3×30 mL), The combined extracts were dried (Na₂SO₄) and concentrated under reduced pressure. The crude residue was purified by silica gel chromatography (0→50% EtOAc in hexanes) to give pure 75 in 51% yield. ¹H NMR (400 MHz, CDCl₃) δ 4.93 (m, 1H), 3.61 (m, 1H), 2.73 (m, 1H), 2.63 (m, 2H), 2.52 (m, 4H), 2.45-2.34 (m, 5H), 2.29 (tr, J=7.5 Hz, 2H), 2.07 (br, 1H), 1.73-1.44 (m, 14), 1.41-1.19 (m, 32H), 0.91-0.84 (tr, 9H).

(ii) Methyl N-hexanoyl-N-methylglycinate (73). To a solution of hexanoic acid (1.3 g, 11.1 mmol) in CH₂Cl₂ (50 mL) was added EDCI·HCl (3.19 g, 16.6 mmol), followed by HOBt

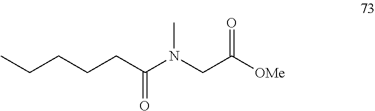

(2.55 g, 16.6 mmol), sarcosine methyl ester hydroxhloride (1.55 g, 11.1 mmol), and diisopropylethylamine (3.9 mL, 22.2 mmol). The mixture was stirred for 16 h under inert atmosphere, then it was quenched with water and extracted with CH₂Cl₂ (3×30 mL). The combined extracts were dried (Na₂SO₄) and concentrated under reduced pressure. The orange residue was purified by column chromatography (0→25% ethyl acetate in hexanes) to give pure 73 in 60% yield. ¹H NMR (400 MHz, CDCl₃, rotamers) δ 4.14-4.00 (s, 2H), 3.79-3.66 (m, 3H), 3.09-2.91 (s, 3H), 2.39-2.16 (tr, 2H), 1.69-1.63 (m, 2H), 1.35-1.25 (m, 4H), 0.93-0.85 (m, 3H).

(iii) N-hexanoyl-N-methylglycine (74). A solution of LiOH·H₂O (1.56 g, 37 mmol) in

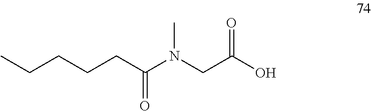

water (10 mL) was added to a solution of 73 (3 g, 15 mmol) in THF OH (30 mL). The mixture was stirred at reflux under inert atmosphere. The reaction was completed in 4 hours, at which point it was cooled, acidified to pH˜1 with 1 M HCl, and extracted with ethyl acetate. The combined extracts were dried under sodium sulfate and concentrated under reduced pressure to afford the product in 90% yield. ¹H NMR (400 MHz, CDCl₃, rotamers) δ 4.20-4.01 (m, 2H), 3.14-2.92 (m, 3H), 2.42-2.19 (m, 2H), 1.70-1.54 (m, 2H), 1.38-1.19 (m, 4H), 0.94-0.83 (m, 3H).

(iv) Exemplary procedure for the esterification of a hydroxy-monoester: 14-((N-hexanoyl-N-methylglycyl)oxy)-1,15-bis(hexylthio)-8-oxopentadecan-2-yl decanoate (76). A solution of alcohol 75 (0.532 g, 0.825 mmol), acid 74 (0.178 g, 0.948 mmol), EDCI·HCl (0.237 g,

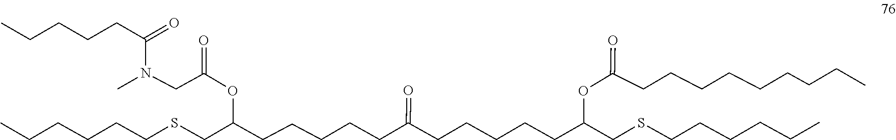 [SMILES: CCCCCCCCCC(=O)OC(CCCCCC(=O)CCCCCC(CSCCCCCC)OC(=O)CN(C)C(=O)CCCCC)CSCCCCCC]

1.24 mmol) and DMAP (0.0705 g, 0.577 mmol) in CH₂Cl₂ (20 mL) was stirred at room temperature for 16 hours, then it was quenched with water and extracted with CH₂Cl₂ (3×30 mL). The combined organics were dried (Na₂SO₄) and concentrated under reduced pressure. The crude residue was purified by silica gel chromatography using a gradient of EtOAc in hexanes to give pure 76 in 75% yield. ¹H NMR (400 MHz, CDCl₃ rotamers) δ 5.10-4.87 (m, 2H), 4.23-4.00 (m, 2H), 3.07 and 2.98 (s, 3H), 2.67-2.59 (m, 4H), 2.56-2.48 (m, 4H), 2.41-2.21 (m, 8H), 1.76-1.49 (m, 16H), 1.40-1.19 (m, 36H), 0.93-0.82 (tr, 12H)

### (D) General Procedure for Silyl Group Release: ((4-Hydroxybutyl)Azanediyl)Bis(1-(pentylthio)heptane-7,2-diyl)bis(decanoate) (5)

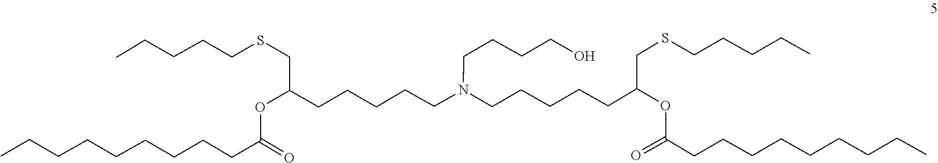 [SMILES: CCCCCCCCCC(=O)OC(CCCCCN(CCCCO)CCCCCC(CSCCCCC)OC(=O)CCCCCCCCC)CSCCCCC]

To a cold (0° C.) solution of TBDPS-protected 46 (1.1 g, 1 mmol) in THF (5 mL) maintained under inert atmosphere was added HF-pyridine (1 mL). The reaction was warmed to room temperature and stirred for 18 hours. Water (10 mL) was added and the mixture was extracted with CH₂Cl₂ (3×15 mL). The combined extracts were dried (Na₂SO₄) and concentrated. The residue was purified by silica chromatography (0-5% MeOH in DCM) to yield lipid 5 (630 mg, 76%) as an oil.

### (E) Preparation of Lipids Having a Type 1 Ionizable Head Group: Synthesis of Lipids 6-11

(i) General procedure for ketone reduction. To a cold (0° C.) solution of a ketone (0.5 mmol) in EtOH (3 mL) was added solid NaBH₄ (0.5 mmol). The mixture was warmed to room temperature and stirred for 30 minutes. The reaction was quenched with sat. aq. NH₄Cl (2 mL), diluted with water (3 mL) and extracted with DCM (3×5 mL). The combined extracts were dried (Na₂SO₄) and concentrated to yield the corresponding alcohol (quantitative) as an oil, which was used in the next step without further purification. The following compounds were thus obtained:

### (ii) 1,13-bis(Cyclohexylthio)-7-hydroxytridecane-2,12-diyl bis(decanoate) (65)

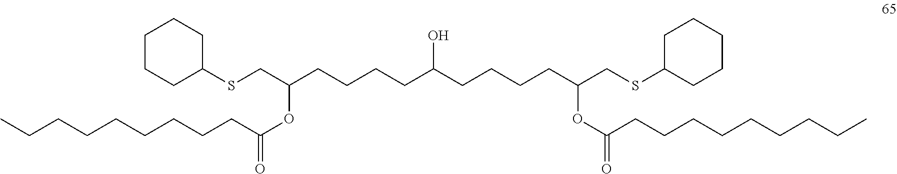 [SMILES: CCCCCCCCCC(=O)OC(CCCCC(O)CCCCC(CSC1CCCCC1)OC(=O)CCCCCCCCC)CSC1CCCCC1]

Obtained from ketone 64. ¹H NMR (400 MHz, CDCl₃) δ 5.00-4.85 (m, 2H), 3.65-3.49 (m, 1H), 2.80-2.60 (m, 6H), 2.30 (t, J=7.5 Hz, 4H), 2.11-1.90 (m, 4H), 1.83-1.14 (m, 60H), 0.98-0.77 (m, 6H).

### (iii) 1,15-bis(Hexylthio)-8-hydroxypentadecane-2,14-diyl bis(decanoate) (70)

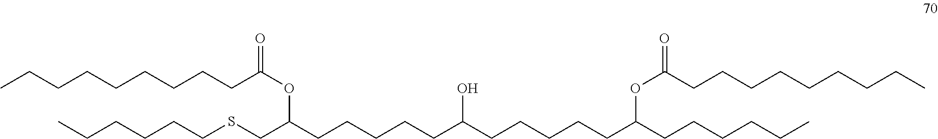 [SMILES: CCCCCCCCCC(=O)OC(CCCCCC)CCCCCC(O)CCCCCC(CSCCCCCC)OC(=O)CCCCCCCCC]

Obtained from ketone 69. ¹H NMR (400 MHz, CDCl₃) δ 5.03-4.88 (m, 2H), 3.60-3.52 (m, 1H), 2.70-2.61 (m, 4H), 2.59-2.49 (m, 4H), 2.30 (t, J=7.5 Hz, 4H), 1.85-1.19 (m, 65H), 0.88 (td, J=6.9, 2.7 Hz, 12H).

(iv) 1,15-bis(Hexylthio)-8-hydroxypentadecane-2,14-diyl bis(3-

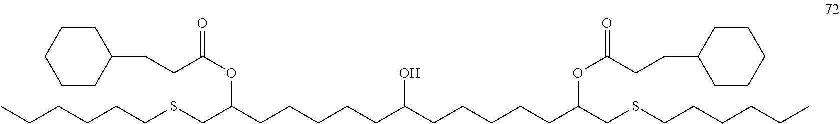 [SMILES: CCCCCCSCC(CCCCCC(O)CCCCCC(CSCCCCCC)OC(=O)CCC1CCCCC1)OC(=O)CCC1CCCCC1]

cyclohexylpropanoate) (72). Obtained from ketone 71. H NMR (400 MHz, CDCl₃) δ 5.00-4.89 (m, 2H), 3.66-3.51 (m, 1H), 2.72-2.59 (m, 4H), 2.53 (td, J=7.2, 1.4 Hz, 4H), 2.39-2.26 (m, 4H), 1.86-1.02 (m, 58H), 0.97-0.81 (m, 10H).

(v) 14-((N-hexanoyl-N-methylglycyl)oxy)-1,15-bis(hexylthio)-8-hydroxypentadecan-2-yl decanoate (77). Obtained from ketone 76. ¹H NMR (400 MHz, CDCl₃, rotamers) δ 5.11-

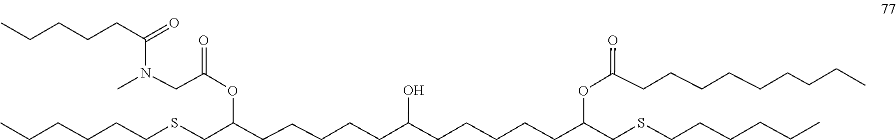 [SMILES: CCCCCCCCCC(=O)OC(CCCCCC(O)CCCCCC(CSCCCCCC)OC(=O)CN(C)C(=O)CCCCC)CSCCCCCC]

### 4.89 (m, 2H), 4.29-3.95 (m, 2H), 3.56 (m, 1H), 3.08 and 2.98 (s, 3H), 2.68-2.60 (m, 4H), 2.57-2.48 (m, 4H), 2.36 (tr, J=7.6, 2H), 2.30 (tr, J=7.5, 7.5, 2H), 1.76-1.50 (m, 13H), 1.45-1.21 (m, 44H), 0.92-0.83 (t, 12H).

(vi) 1,15-bis(Cyclohexylthio)-8-hydroxypentadecane-2,14-diyl bis(decanoate) (80). Obtained from ketone 79. H NMR (400 MHz, CDCl₃) δ 4.98-4.86 (m, 2H), 3.63-3.53 (m, 1H),

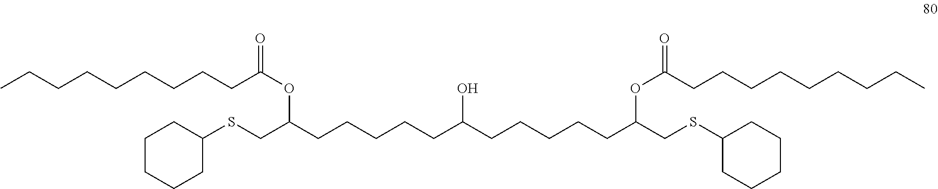 [SMILES: CCCCCCCCCC(=O)OC(CCCCCC(O)CCCCCC(CSC1CCCCC1)OC(=O)CCCCCCCCC)CSC1CCCCC1]

### 2.77-2.55 (m, 6H), 2.30 (t, J=7.5 Hz, 4H), 2.04-1.90 (m, 4H), 1.82-1.18 (m, 64H), 0.94-0.81 (m, 6H).

(vii) 1,15-bis(Cyclohexylthio)-8-hydroxypentadecane-2,14-diyl dinonanoate (82). Obtained from ketone 81. ¹H NMR (400 MHz, CDCl₃) δ 4.98-4.88 (m, 2H), 3.63-3.51 (m, 1H),

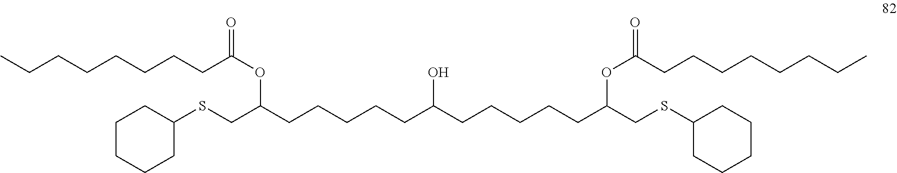 [SMILES: CCCCCCCCC(=O)OC(CCCCCC(O)CCCCCC(CSC1CCCCC1)OC(=O)CCCCCCCC)CSC1CCCCC1]

### 2.80-2.57 (m, 6H), 2.29 (t, J=7.5 Hz, 4H), 1.96 (dd, J=8.8, 5.0 Hz, 4H), 1.84-1.16 (m, 60H), 0.93-0.82 (m, 6H).

(viii) General procedure for alcohol esterification. A solution of an alcohol (0.5 mmol, 1 equiv), 4-(dimethylamino)butyric acid hydrochloride (0.65 mmol, 1.3 equiv), EDCI·HCl (0.7 mmol, 1.4 equiv) and DMAP (23.1 mg, 0.7 mmol, 1.4 equiv) in DCM (5 mL) was stirred under inert atmosphere at room temperature for 18 hours then concentrated. The residue was purified by silica chromatography (0-5% MeOH in DCM) to yield the lipid (75-85%) as an oil. The following lipids were thus obtained:

(ix) 1,13-bis(Cyclohexylthio)-7-((4-(dimethylamino)butanoyl)oxy)tridecane-2,12-diyl bis-(decanoate) (6). Obtained from alcohol 65. ¹H NMR (400 MHz, C₆D₆) δ 5.29-5.17 (m,

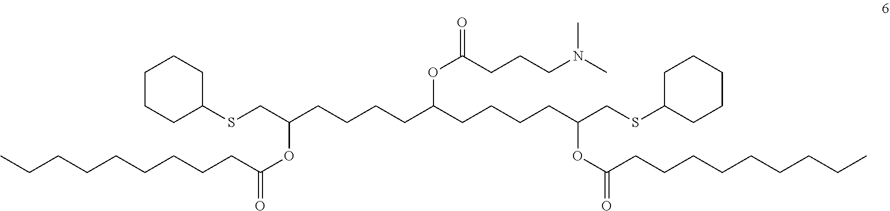 [SMILES: CCCCCCCCCC(=O)OC(CCCCC(CCCCC(CSC1CCCCC1)OC(=O)CCCCCCCCC)OC(=O)CCCN(C)C)CSC1CCCCC1]

### 2H), 5.14-5.04 ON (m, 1H), 2.81-2.58 (m, 6H), 2.43-2.35 (m, 2H) 2.29 (td, J=7.4, 1.9 Hz, 4H), 2.17 (t, J=6.8 Hz, 2H), 2.07 (s, 6H), 2.05-1.94 (m, 4H), 1.90-1.02 (m, 62H), 0.92 (t, J=6.9 Hz, 6H).

(x) 8-((4-(Dimethylamino)butanoyl)oxy)-1,15-bis(hexylthio)pentadecane-2,14-diyl bis(decanoate) (7). Obtained from alcohol 70. ¹H NMR (400 MHz, CDCl₃) δ 4.99-4.90 (m,

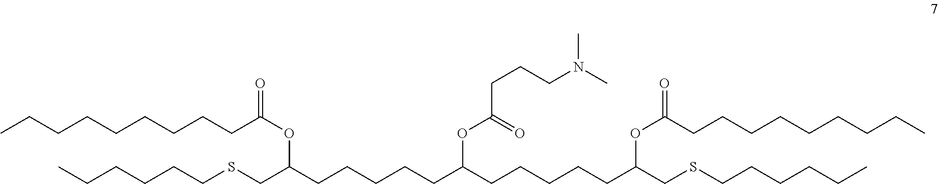 [SMILES: CCCCCCCCCC(=O)OC(CCCCCC(CCCCCC(CSCCCCCC)OC(=O)CCCCCCCCC)OC(=O)CCCN(C)C)CSCCCCCC]

### 2H), 4.84 (p, J=6.4 Hz, 1H), 2.70-2.59 (m, 4H), 2.53 (td, J=7.3, 1.4 Hz, 4H), 2.42-2.22 (m, 14H), 1.92-1.19 (m, 66H), 0.88 (td, J=6.9, 2.6 Hz, 12H).

(xi) 8-((4-(Dimethylamino)butanoyl)oxy)-1,15-bis(hexylthio)pentadecane-2,14-diyl bis(3-cyclohexylpropanoate) (8). Obtained from alcohol 72. ¹H NMR (400 MHz, C₆D₆) δ 5.33-

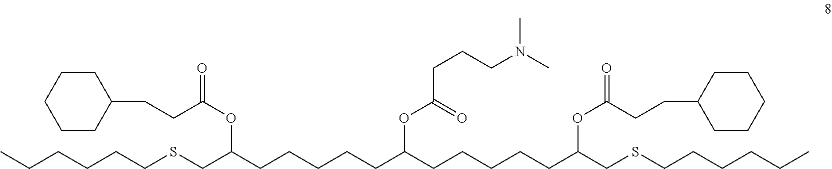 [SMILES: CCCCCCSCC(CCCCCC(CCCCCC(CSCCCCCC)OC(=O)CCC1CCCCC1)OC(=O)CCCN(C)C)OC(=O)CCC1CCCCC1]

### 5.18 (m, 2H), 5.16-5.05 (m, 1H), 2.77-2.43 (m, 8H), 2.37 (t, J=7.3 Hz, 2H), 2.33-2.27 (m, 4H), 2.16 (t, J=6.9 Hz, 2H), 2.05 (s, 6H), 1.89-0.98 (m, 60H), 0.87 (t, J=7.0 Hz, 6H), 0.83-0.72 (m, 4H).

(xii) 8-((4-(dimethylamino)butanoyl)oxy)-14-((N-hexanoyl-N-methylglycyl)oxy)-

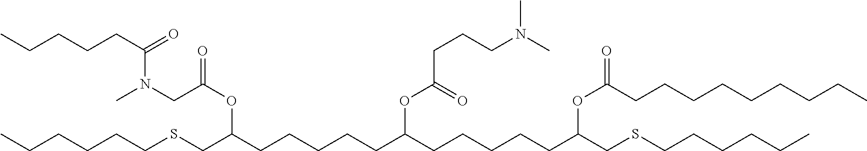 [SMILES: CCCCCCCCCC(=O)OC(CCCCCC(CCCCCC(CSCCCCCC)OC(=O)CN(C)C(=O)CCCCC)OC(=O)CCCN(C)C)CSCCCCCC]

1,15-bis-(hexylthio)pentadecan-2-yl decanoate (9). Obtained from alcohol 77. ¹H NMR (400 MHz, CDCl₃, rotamers) δ 5.09-4.89 (m, 2H), 4.84 (m, 1H), 4.23-4.02 (m, 2H), 3.07 and 2.98 (s, 3H), 2.68-2.60 (m, 4H), 2.56-2.46 (m, 4H), 2.40-2.21 (m, 12H), 1.81 (m, 2H), 1.76-1.42 (m, 18H), 1.42-1.17 (m, 40H), 0.93-0.83 (tr, J=7.1 Hz, 12H).

(xiii) 1,15-bis(cyclohexylthio)-8-((4-(dimethylamino)butanoyl)oxy)pentadecane-2,14-diyl bis(decanoate) (10). Obtained from alcohol 80. ¹H NMR (400 MHz, CDCl₃) δ 4.98-

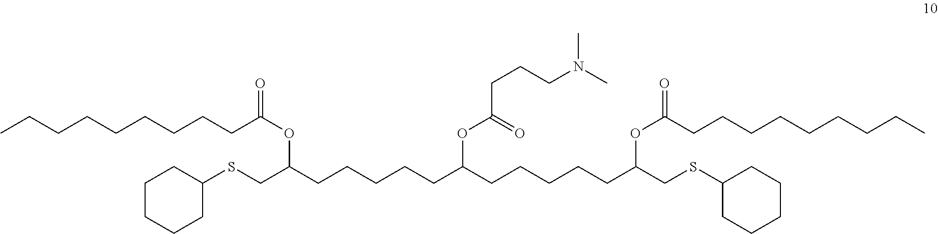 [SMILES: CCCCCCCCCC(=O)OC(CCCCCC(CCCCCC(CSC1CCCCC1)OC(=O)CCCCCCCCC)OC(=O)CCCN(C)C)CSC1CCCCC1]

### 4.87 (m, 2H), 4.87-4.79 (m, 1H), 2.74-2.59 (m, 6H), 2.53-2.22 (m, 14H), 2.06-1.42 (m, 28H), 1.28 (d, J=9.3 Hz, 42H), 0.92-0.83 (m, 6H).

(xiv) 1,15-bis(cyclohexylthio)-8-((4-(dimethylamino)butanoyl)oxy)pentadecane-2,14-diyl dinonanoate (11). Obtained from alcohol 82. ¹H NMR (400 MHz, CDCl₃) δ 4.95-4.87

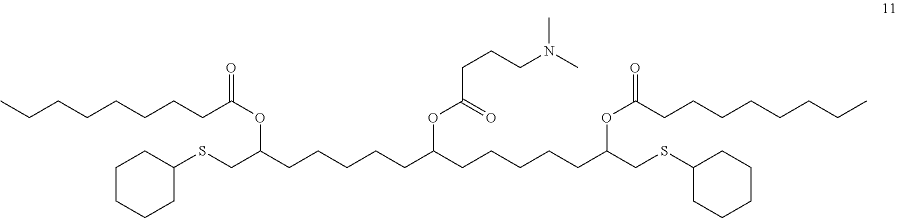 [SMILES: CCCCCCCCC(=O)OC(CCCCCC(CCCCCC(CSC1CCCCC1)OC(=O)CCCCCCCC)OC(=O)CCCN(C)C)CSC1CCCCC1]

### (m, 2H), 4.87-4.79 (m, 1H), 2.73-2.57 (m, 6H), 2.35-2.25 (m, 8H), 2.22 (s, 6H), 2.04-1.90 (m, 4H), 1.88-1.41 (m, 20H), 1.36-1.20 (m, 42H), 0.92-0.83 (m, 6H).

### (F) Preparation of Lipids Having a Ketal-Type Ionizable Head Group: Synthesis of Lipids 12-21

(i) General procedure for ketone ketalization. A solution of a ketone (1 mmol, 1 equiv), a diol (2 mmol, 2 equiv) and pyridinium p-toluenesulfonate (PPTS, 0.2 mmol, 0.2 equiv) in toluene (10.0 mL) was refluxed under nitrogen with continuous removal of water (Dean-Stark trap) until TLC and NMR indicated complete conversion to the product (12 h-4 days depending on the diol). The mixture was cooled to room temperature, washed with water (2×10 mL) and brine (10 mL), dried (Na₂SO₄) and concentrated. The residue was purified by silica gel column chromatography (0-2% MeOH in CH₂Cl₂) to yield the ketal (65-85%) as an oil. The following compounds were thus obtained:

(ii) (4-(2-Hydroxyethyl)-1,3-dioxolane-2,2-diyl)bis(1-(pentylthio)hexane-6,2-diyl)bis(decan-oate) (85). Obtained from ketone 84 and 1,2,4-butanetriol. H NMR (400 MHz,

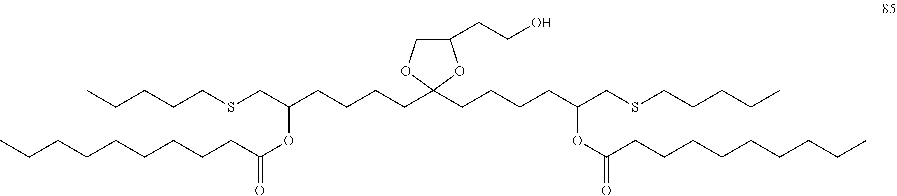 [SMILES: CCCCCCCCCC(=O)OC(CCCCC1(CCCCC(CSCCCCC)OC(=O)CCCCCCCCC)OCC(CCO)O1)CSCCCCC]

### CDCl₃) δ 5.01-4.89 (in, 2H), 4.28-4.18 (m, 1H), 4.07 (t, J=7.2 Hz, 1H), 3.83-3.77 (m, 2H), 3.51 (td, J=8.1, 2.8 Hz, 1H), 2.71-2.58 (m, 4H), 2.53 (td, J=7.3, 1.4 Hz, 4H), 2.30 (t, J=7.5 Hz, 4H), 2.02-1.16 (m, 58H), 0.88 (q, J=7.0 Hz, 12H).

(iii) (4-(bromomethyl)-1,3-dioxolane-2,2-diyl)bis(1-(pentylthio)hexane-6,2-diyl)bis(decano-ate) (87). Obtained from ketone 84 and 3-bromo-1,2-propanediol. ¹H NMR (400

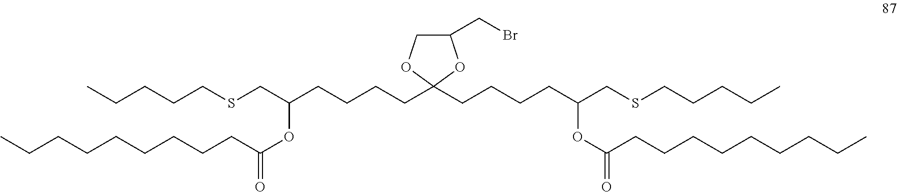 [SMILES: CCCCCCCCCC(=O)OC(CCCCC1(CCCCC(CSCCCCC)OC(=O)CCCCCCCCC)OCC(CBr)O1)CSCCCCC]

MHz, CDCl₃) δ5.02-4.86 (m, 2H), 4.37-4.26 (m, 1H), 4.13 Hz, 1H), 3.76 (dd, J=8.6, 6.1 Hz, 1H), 3.44 (dd, J=10.0, 4.6 Hz, 1H), 3.29 (dd, J=10.0, 8.1 Hz, 1H), 2.71-2.57 (m, 4H), 2.57-2.51 (m, 4H), 2.30 (t, J=7.5 Hz, 4H), 1.84-1.16 (m, 56H), 1.03-0.80 (m, 12H).

(iv) (4-(2-Hydroxyethyl)-1,3-dioxolane-2,2-diyl)bis(1-(cyclohexylthio)hexane-6,2-diyl)bis-(decanoate) (88). Obtained from ketone 64 and 1,2,4-butanetriol. H NMR (400 MHz,

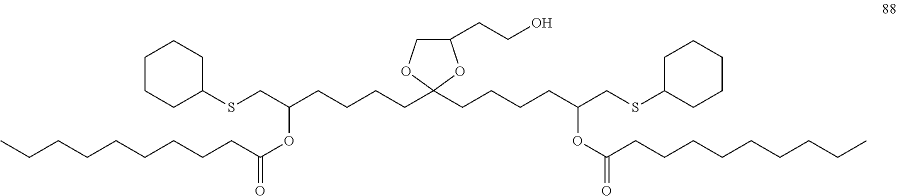 [SMILES: CCCCCCCCCC(=O)OC(CCCCC1(CCCCC(CSC2CCCCC2)OC(=O)CCCCCCCCC)OCC(CCO)O1)CSC1CCCCC1]

### CDCl₃) δ 4.99-4.84 (m, 2H), 4.32-4.15 (m, 1H), 4.11-4.03 (m, 1H), 3.79 (t, J=5.6 Hz, 2H), 3.50 (td, J=8.0, 2.6 Hz, 1H), 2.78-2.58 (m, 6H), 2.29 (t, J=7.5 Hz, 4H), 2.03-1.11 (m, 66H), 0.96-0.81 (m, 6H).

(v) (5,5-bis(Hydroxymethyl)-1,3-dioxane-2,2-diyl)bis(1-(cyclohexylthio)hexane-6,2-diyl)bis-(decanoate) (90). Obtained from ketone 64 and pentaerythritol. ¹H NMR (400 MHz,

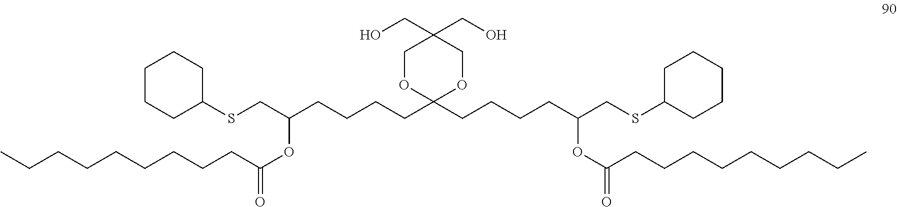 [SMILES: CCCCCCCCCC(=O)OC(CCCCC1(CCCCC(CSC2CCCCC2)OC(=O)CCCCCCCCC)OCC(CO)(CO)CO1)CSC1CCCCC1]

### CDCl₃) δ 5.01-4.87 (m, 2H), 3.75 (s, 4H), 3.70 (s, 4H), 2.79-2.59 (m, 6H), 2.30 (t, J=7.6 Hz, 4H), 2.01-1.88 (m, 4H), 1.85-1.15 (m, 60H), 0.88 (t, J=6.7 Hz, 6H).

(vi) (4-(Bromomethyl)-1,3-dioxolane-2,2-diyl)bis(1-(hexylthio)heptane-7,2-diyl)bis(decano-ate) (91). Obtained from ketone 69 and 3-bromo-1,2-propanediol. ¹H NMR (400

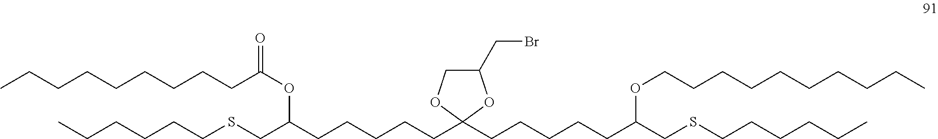 [SMILES: CCCCCCCCCCOC(CCCCCC1(CCCCCC(CSCCCCCC)OC(=O)CCCCCCCCC)OCC(CBr)O1)CSCCCCCC]

MHz, CDCl₃) δ 5.01-4.88 (m, 2H), 4.37-4.28 (m, 1H), 4.13 (dd, J=8.5, 6.2 Hz, 1H), 3.77 (dd, J=8.6, 6.1 Hz, 1H), 3.45 (dd, J=10.0, 4.6 Hz, 1H), 3.29 (dd, J=10.0, 8.1 Hz, 1H), 2.73-2.59 (m, 4H), 2.53 (td, J=7.3, 1.4 Hz, 4H), 2.30 (t, J=7.5 Hz, 4H), 1.75-1.49 (m, 18H), 1.45-1.20 (m, 46H), 0.88 (m, 12H).

(vii) (5-(hydroxymethyl)-1,3-dioxane-2,2-diyl)bis(1-(hexylthio)heptane-7,2-diyl)bis(decano-ate) (92). Obtained from ketone 69 and 2-hydroxymethyl-1,3-propanediol. ¹H NMR

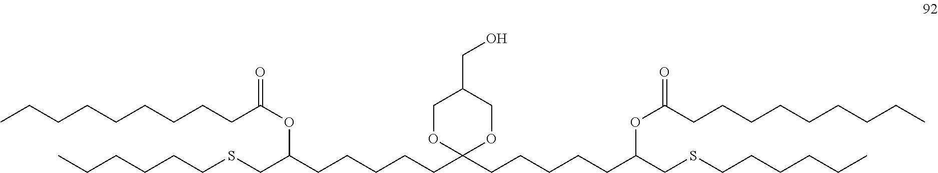 [SMILES: CCCCCCCCCC(=O)OC(CCCCCC1(CCCCCC(CSCCCCCC)OC(=O)CCCCCCCCC)OCC(CO)CO1)CSCCCCCC]

### (400 MHz, CDCl₃) δ 5.00-4.91 (m, 2H), 3.98 (dd, J=11.8, 4.0 Hz, 2H), 3.80-3.71 (m, 4H), 2.70-2.60 (m, 4H), 2.53 (td, J=7.3, 1.4 Hz, 4H), 2.30 (t, J=7.5 Hz, 4H), 1.61 (m, 19H), 1.44-1.19 (m, 46H), 0.99-0.83 (m, 12H).

(viii) General procedure for hydroxyketal tosylation. Solid TsCl (1.3 mmol, 1.3 equiv) was added to a cold (0° C.) CH₂Cl₂ (3 mL) solution of a hydroxyketal (1 mmol, 1 equiv), Et₃N (1.5 mmol, 1.5 equiv) and DMAP (0.1 mmol, 0.1 equiv) under nitrogen atmosphere. The reaction was warmed to room temperature and stirred until TLC and ¹H NMR indicated complete conversion to the product. The reaction was quenched with water (10 mL) and extracted with CH₂Cl₂ (3×10 mL). The combined extracts were dried (Na₂SO₄) and concentrated to yield the crude tosylate (˜quant.), which was used in the next step without purification. The following compounds were thus obtained:

(ix) (4-(2-(Tosyloxy)ethyl)-1,3-dioxolane-2,2-diyl)bis(1-(pentylthio)hexane-6,2-diyl)bis(decanoate) (86). Obtained from hydroxyketal 85. ¹H NMR (400 MHz, CDCl₃) δ 7.79 (d, J=8.3 Hz, 2H), 7.35 (d, J=8.1 Hz, 2H), 5.01-4.88 (m, 2H), 4.19-4.04 (m, 3H), 4.03-3.98 (m, 1H),

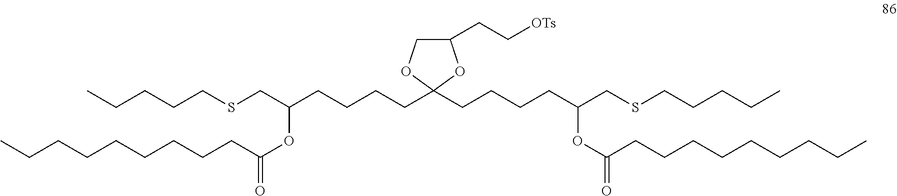 [SMILES: CCCCCCCCCC(=O)OC(CCCCC1(CCCCC(CSCCCCC)OC(=O)CCCCCCCCC)OCC(CCOS(=O)(=O)c2ccc(C)cc2)O1)CSCCCCC]

### 3.43 (t, J=7.8 Hz, 1H), 2.71-2.58 (m, 4H), 2.57-2.51 (m, 4H), 2.46 (s, 3H), 2.29 (td, J=7.6, 2.2 Hz, 4H), 1.96-1.18 (m, 58H), 0.94-0.83 (m, 12H).

(x) (4-(2-(tosyloxy)ethyl)-1,3-dioxolane-2,2-diyl)bis(1-(cyclohexylthio)hexane-6,2-diyl)bis-(decanoate) (89). Obtained from hydroxyketal 88. ¹H NMR (400 MHz, CDCl₃) δ 7.79

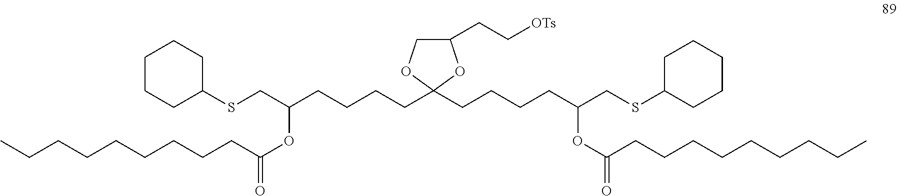 [SMILES: CCCCCCCCCC(=O)OC(CCCCC1(CCCCC(CSC2CCCCC2)OC(=O)CCCCCCCCC)OCC(CCOS(=O)(=O)c2ccc(C)cc2)O1)CSC1CCCCC1]

### (d, J=8.3 Hz 7.8 Hz, 2H), 4.98-4.86 (m, (m, 3H), 4.04-3.97 (m, 1H), 3.43 (t, J=7.7 Hz, 1H), 2.75-2.56 (m, 6H), 2.45 (s, 3H), 2.34-2.22 (m, 4H), 2.06-1.04 (m, 66H), 0.95-0.81 (m, 6H).

(xi) (5-((Tosyloxy)methyl)-1,3-dioxane-2,2-diyl)bis(1-(hexylthio)heptane-7,2-diyl)bis-(decanoate) (93). H NMR (400 MHz, CDCl₃) δ 7.80 (d, J=8.2 Hz, 2H), 7.36 (d, J=8.0

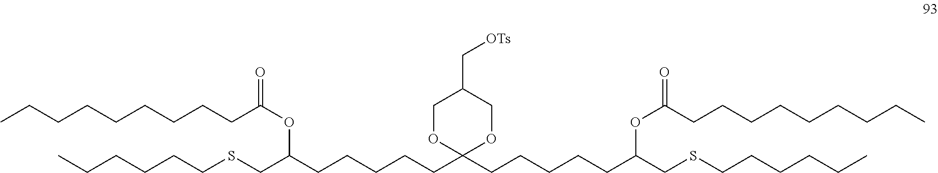 [SMILES: CCCCCCCCCC(=O)OC(CCCCCC1(CCCCCC(CSCCCCCC)OC(=O)CCCCCCCCC)OCC(COS(=O)(=O)c2ccc(C)cc2)CO1)CSCCCCCC]

Hz, 2H), 5.03-4.87 (m, 2H) 4.17 (d, J=7.2 Hz, 2H), 3.95 (dd, J=11.9, 3.6 Hz, 2H), 3.64 (dd, J=12.2, 4.0 Hz, 2H), 2.69-2.59 (m, 4H), 2.54 (ddt, J=8.2, 6.1, 2.5 Hz, 4H), 2.45 (s, 3H), 2.33-2.27 (m, 4H), 1.94-1.15 (m, 65H), 0.93-0.83 (m, 12H).

(xii) Exemplary procedure for bromide/tosylate displacement with a low-boiling amine: (4-(2-(dimethylamino)ethyl)-1,3-dioxolane-2,2-diyl)bis(1-(pentylthio)hexane-6,2-diyl)bis-(decanoate) (12). A solution of tosylate 86 (49.0 mg, 0.0481 mmol), dimethyl amine (2 M in THF, 1 mL) and MeOH (1 mL) was heated in a microwave reactor (110° C., normal absorbance) for 15 minutes. The mixture was then concentrated, and the residue purified by

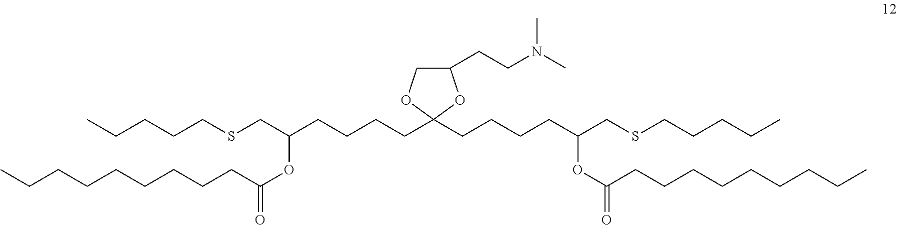 [SMILES: CCCCCCCCCC(=O)OC(CCCCC1(CCCCC(CSCCCCC)OC(=O)CCCCCCCCC)OCC(CCN(C)C)O1)CSCCCCC]

silica chromatography (0-5% MeOH in DCM) to yield lipid 12 (31 mg, 74%) as an oil. ¹H NMR (400 MHz, C₆D₆) δ 5.31-5.20 (m, 2H), 4.13-4.01 (m, 1H), 3.93 (dd, J=7.7, 5.9 Hz, 1H), 3.41 (t, J=7.9 Hz, 1H), 2.74-2.41 (m, 8H), 2.33-2.17 (m, 6H), 2.05 (s, 6H), 1.84-1.12 (m, 58H), 1.01-0.81 (m, 12H).

(xiii) Exemplary procedure for bromide/tosylate displacement with a high-boiling amine: (4-(((4-hydroxybutyl)(methyl)amino)methyl)-1,3-dioxolane-2,2-diyl)bis(1-(pentylthio)hexane-6,2-diyl)bis(decanoate) (13). A mixture of bromoketal 87 (75 mg, 0.0854 mmol), 4-(methylamino)-1-butanol (11 mg, 0.111 mmol) and K₂CO₃ (15.3 mg, 0.111 mmol) in

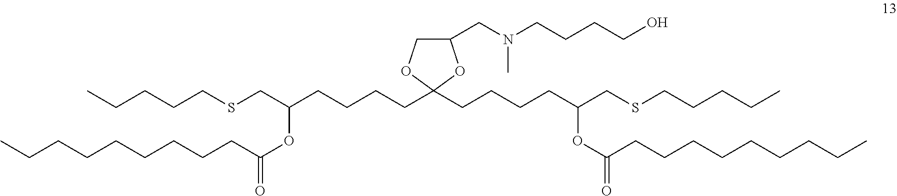 [SMILES: CCCCCCCCCC(=O)OC(CCCCC1(CCCCC(CSCCCCC)OC(=O)CCCCCCCCC)OCC(CN(C)CCCCO)O1)CSCCCCC]

MeCN (1 mL) was stirred at 80° C. in a sealed reaction vessel for 18 hours. The mixture was cooled, diluted with water (2 mL) and extracted with CH₂Cl₂ (3×3 mL). The combined extracts were dried (Na₂SO₄) and concentrated. The residue was purified by silica chromatography (0-5% MeOH in CH₂Cl₂) to yield lipid 13 (49 mg, 64%) as an oil. ¹H NMR (400 MHz, C₆D₆) δ 5.31-5.19 (m, 2H), 4.27-4.18 (m, 1H), 3.96 (dd, J=8.0, 6.2 Hz, 1H), 3.66-3.52 (m, 2H), 3.47 (t, J=7.9 Hz, 1H), 2.72-2.41 (m, 9H), 2.34-2.23 (m, 6H), 2.18-2.10 (m, 1H), 2.09 (s, 3H), 1.82-1.17 (m, 60H), 0.92 (t, J=6.9 Hz, 6H), 0.85 (t, J=7.0 Hz, 6H).

(xiv) (4-(2-(Ethyl(4-hydroxybutyl)amino)ethyl)-1,3-dioxolane-2,2-diyl)bis(1-(pentylthio)-hexane-6,2-diyl)bis(decanoate) (14). Prepared from bromoketal 87 and 4-

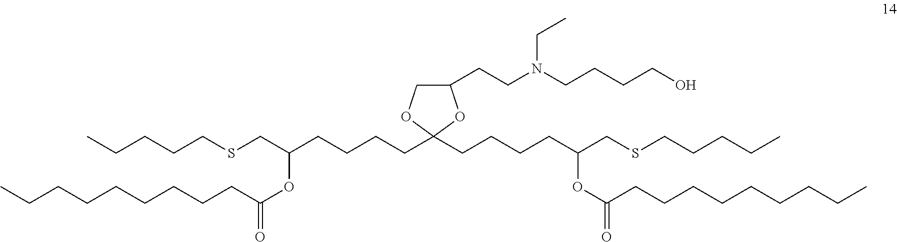 [SMILES: CCCCCCCCCC(=O)OC(CCCCC1(CCCCC(CSCCCCC)OC(=O)CCCCCCCCC)OCC(CCN(CC)CCCCO)O1)CSCCCCC]

(ethylamino)-1-butanol by procedure (xiii) above. ¹H NMR (400 MHz, C₆D₆) δ 5.30-5.19 (m, 2H), 4.06-3.94 (m, 1H), 3.88 (t, J=6.8 Hz, 1H), 3.62 (t, J=5.3 Hz, 2H), 3.37 (t, J=7.8 Hz, 1H), 2.80-2.12 (m, 18H), 1.82-1.17 (m, 62H), 0.96-0.89 (m, 9H), 0.88=0.82 (m, 6H).

(xv) (4-(2-(4-Hydroxypiperidin-1-yl)ethyl)-1,3-dioxolane-2,2-diyl)bis(1-(pentylthio)hexane-6,2-diyl)bis(decanoate) (15). Prepared from bromoketal 87 and 4-

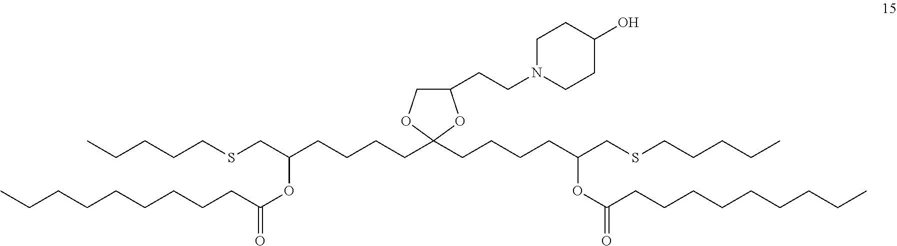 [SMILES: CCCCCCCCCC(=O)OC(CCCCC1(CCCCC(CSCCCCC)OC(=O)CCCCCCCCC)OCC(CCN2CCC(O)CC2)O1)CSCCCCC]

piperidinol by procedure (xiii) above. ¹H NMR (400 MHz, C₆D₆) δ 5.32-5.17 (m, 2H), 4.12-4.02 (m, 1H), 3.99-3.92 (m, 1H), 3.53-3.29 (m, 2H), 2.73-2.41 (m, 10H), 2.38-2.17 (m, 8H), 1.96-1.15 (m, 62H), 0.92 (t, J=6.9 Hz, 6H), 0.85 (t, J=6.9 Hz, 6H).

(xvi) (4-(2-(Dimethylamino)ethyl)-1,3-dioxolane-2,2-diyl)bis(1-(cyclohexylthio)hexane-6,2-diyl)bis(decanoate) (16). Prepared from tosylate 89 and

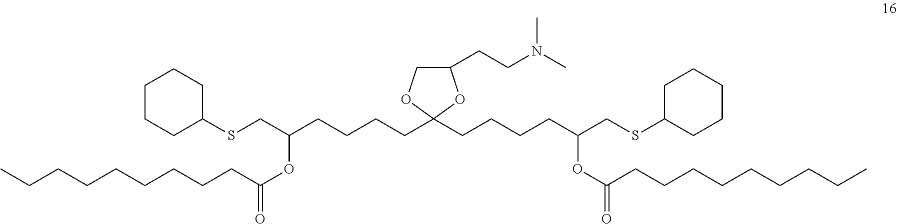 [SMILES: CCCCCCCCCC(=O)OC(CCCCC1(CCCCC(CSC2CCCCC2)OC(=O)CCCCCCCCC)OCC(CCN(C)C)O1)CSC1CCCCC1]

dimethylamine by procedure (xii) above. ¹H NMR (400 MHz, CDCl₃) δ 7.79 (d, J=8.3 Hz, 2H), 7.35 (d, J=7.8 Hz, 2H), 4.98-4.86 (m, 2H), 4.26-4.05 (m, 3H), 4.04-3.97 (m, 1H), 3.43 (t, J=7.7 Hz, 1H), 2.75-2.56 (m, 6H), 2.45 (s, 3H), 2.34-2.22 (m, 4H), 2.06-1.04 (m, 66H), 0.95-0.81 (m, 6H).

(xvii) (5-(((3-(Dimethylamino)propanoyl)oxy)methyl)-5-(hydroxymethyl)-1,3-dioxane-2,2-diyl)bis(1-(cyclohexylthio)hexane-6,2-diyl)bis(decanoate) (17). Prepared from dihydroxyketal 90 and 3-(dimethylamino)propanoic acid hydrochloride by procedure (viii) of part E above, except that 0.8 equivalents acid and 1 equivalent EDCI·HCl/DMAP were used. ¹H

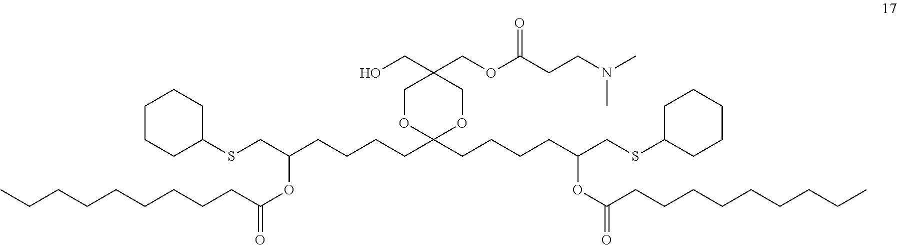 [SMILES: CCCCCCCCCC(=O)OC(CCCCC1(CCCCC(CSC2CCCCC2)OC(=O)CCCCCCCCC)OCC(CO)(COC(=O)CCN(C)C)CO1)CSC1CCCCC1]

NMR (400 MHz, C₆D₆) δ 5.37-5.15 (m, 2H) 4.39 (s, 2H), 3.79-3.67 (m, 4H), 3.55 (s, 2H), 2.83-2.61 (m, 6H), 2.32-2.26 (m, 4H), 2.23 (t, J=6.4 Hz, 2H), 2.11 (t, J=6.4 Hz, 2H), 2.06-1.94 (m, 4H), 1.91 (s, 6H), 1.83-1.04 (m, 60H), 0.92 (t, J=6.9 Hz, 6H).

(xviii) (5-(((4-(Dimethylamino)butanoyl)oxy)methyl)-5-(hydroxymethyl)-1,3-dioxane-2,2-diyl)bis(1-(cyclohexylthio)hexane-6,2-diyl)bis(decanoate) (18). Prepared from dihydroxyketal 90 and 4-(dimethylamino)butanoic acid hydrochloride by procedure (viii) of part E above, except that 0.8 equivalents acid and 1 equivalent EDCI·HCl/DMAP were used. ¹H

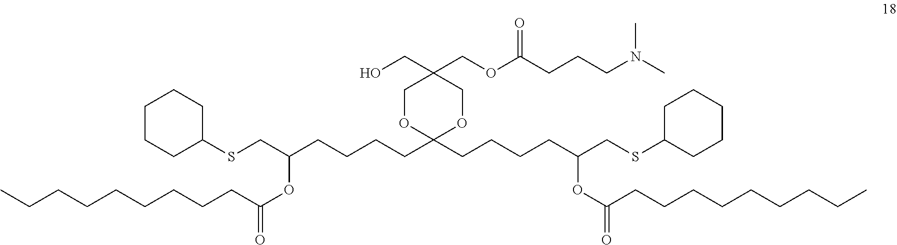 [SMILES: CCCCCCCCCC(=O)OC(CCCCC1(CCCCC(CSC2CCCCC2)OC(=O)CCCCCCCCC)OCC(CO)(COC(=O)CCCN(C)C)CO1)CSC1CCCCC1]

NMR (400 MHz, C₆D₆) δ 5.39-5.21 (m, 2H), 4.33 (s, 2H), 3.81-3.68 (m, 4H), 3.47 (s, 2H), 2.81-2.61 (m, 6H), 2.28 (t, J=7.3 Hz, 4H), 2.16 (t, J=7.1 Hz, 2H), 2.10-1.95 (m, 12H), 1.84-1.05 (m, 62H), 0.92 (t, J=6.9 Hz, 6H).

(xix) (4-((Dimethylamino)methyl)-1,3-dioxolane-2,2-diyl)bis(1-(hexylthio)heptane-7,2-diyl)bis(decanoate) (19). Prepared from bromoketal 91 by procedure (xii) above). ¹H NMR

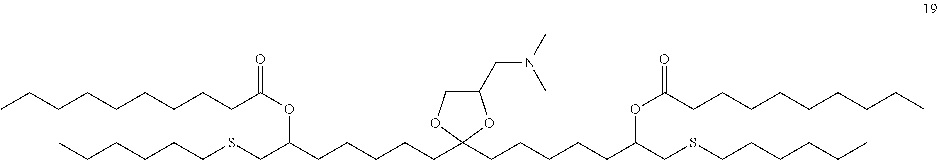 [SMILES: CCCCCCCCCC(=O)OC(CCCCCC1(CCCCCC(CSCCCCCC)OC(=O)CCCCCCCCC)OCC(CN(C)C)O1)CSCCCCCC]

### (400 MHz, CDCl₃) δ 5.05-4.87 (m, 2H), 4.22 (p, J=6.5 Hz, 1H), 4.07 (dd, J=7.9, 6.2 Hz, 1H), 3.50 (t, J=7.8 Hz, 1H), 2.71-2.59 (m, 4H), 2.59-2.45 (m, 5H), 2.43-2.23 (m, 11H), 1.79-1.46 (m, 18H), 1.47-1.15 (m, 46H), 0.88 (m, 12H).

(xx) (4-(((4-Hydroxybutyl)(methyl)amino)methyl)-1,3-dioxolane-2,2-diyl)bis(1-(hexylthio)-heptane-7,2-diyl)bis(decanoate) (20). Prepared from bromoketal 91 and 4-(methylamino)-1-butanol by procedure (xiii) above. H NMR (400 MHz, CDCl₃) δ 5.04-4.89 (m,

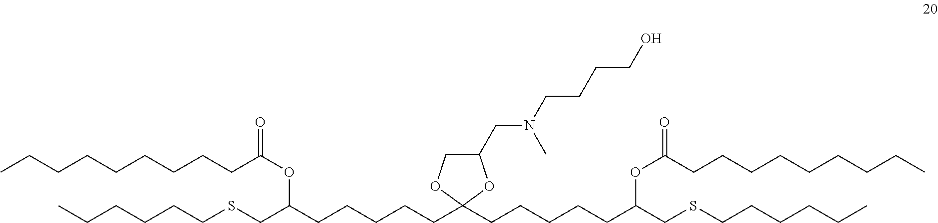 [SMILES: CCCCCCCCCC(=O)OC(CCCCCC1(CCCCCC(CSCCCCCC)OC(=O)CCCCCCCCC)OCC(CN(C)CCCCO)O1)CSCCCCCC]

### 2H), 4.35-4.19 (m, 1H), 4.10 (dd, J=8.06, 6.2 Hz, 1H), 3.67-3.55 (m, 2H), 3.49 (t, J=7.9 Hz, 1H), 2.70-2.41 (m, 12H), 2.38-2.24 (m, 7H), 1.77-1.47 (m, 20H), 1.44-1.18 (m, 48H), 0.95-0.82 (m, 12H).

(xxi) (5-(((4-Hydroxybutyl)(methyl)amino)methyl)-1,3-dioxane-2,2-diyl)bis(1-(hexylthio)-heptane-7,2-diyl)bis(decanoate) (21). Prepared from tosylate 93 and 4-(methylamino)-1-butanol by procedure (xiii) above. H NMR (400 MHz, C₆D₆) δ 5.36-5.16 (m,

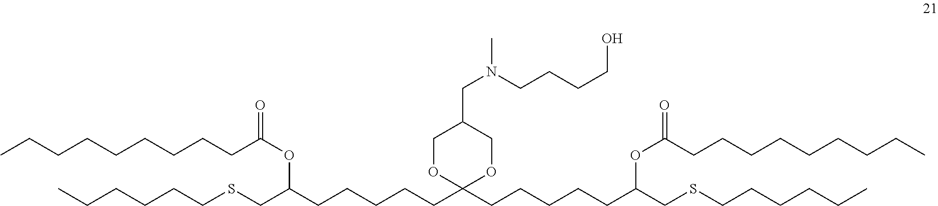 [SMILES: CCCCCCCCCC(=O)OC(CCCCCC1(CCCCCC(CSCCCCCC)OC(=O)CCCCCCCCC)OCC(CN(C)CCCCO)CO1)CSCCCCCC]

### 2H), 3.91 (dd, J=11.7, 4.0 Hz, 2H), 3.65 (dd, J=11.7, 6.4 Hz, 2H), 3.55 (t, J=5.4 Hz, 2H), 2.75-2.42 (m, 8H), 2.37-2.20 (m, 4H), 2.16 (d, J=7.3 Hz, 2H), 2.09 (t, J=6.0 Hz, 2H), 1.90 (s, 3H), 1.88-1.12 (m, 69H), 0.99-0.83 (m, 12H).

### (G) Synthesis of Precursors of Lipids Having a Type 7 Ionizable Head Group (Lipids 22-35)

(i) General procedure for reductive amination of a ketone with a primary amine leading to a secondary amine. To a solution of a ketone (1 mmol) and a primary amine (2 mmol, 2 equiv) in 1,2-dichloroethane (10 mL) was added NaBH(OAc)₃ (1.8 mmol, 1.8 equiv) and HOAc (0.1 mL). The resulting mixture was stirred under nitrogen at room temperature for 18 hours then quenched with sat. aq. NaHCO₃ (3 mL), diluted with water (5.00 mL) and extracted with DCM (3×10 mL). The combined extracts were dried (Na₂SO₄) and concentrated. The residue was purified by silica chromatography (0-5% MeOH in DCM) to yield the secondary amine (70-75%) as an oil. The following compounds were thus obtained:

(ii) 7-((4-((tert-Butyldiphenylsilyl)oxy)butyl)amino)-1,13-bis(pentylthio)tridecane-2,12-diyl bis(decanoate) (94). Obtained from ketone 84 and OTBDPS-protected 4-amino-1-

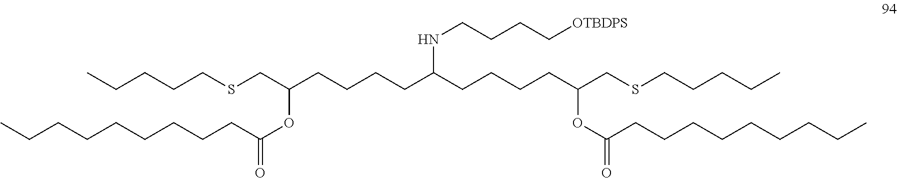

butanol 40. ¹H NMR (400 MHz, CDCl₃) δ 7.77-7.52 (m 4H), 7.45-7.34 (m, 6H), 5.03-4.85 (m, 2H), 3.70-3.63 (m, 2H), 2.71-2.39 (m, 11H), 2.30 (t, J=7.5 Hz, 4H), 1.88-1.18 (m, 60H), 1.04 (s, 9H), 0.97-0.79 (m, 12H).

(iii) 7-((4-((tert-butyldiphenylsilyl)oxy)butyl)amino)-1,13-bis(heptylthio)tridecane-2,12-diyl bis(decanoate) (98). Obtained from ketone 97 and OTBDPS-protected 4-amino-1-butanol 40. ¹H NMR (400 MHz, CDCl₃) δ 7.72-7.59 (m, 4H), 7.46-7.34 (m, 6H), 5.02-4.82 (m,

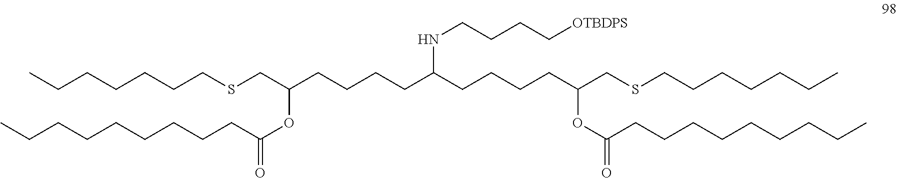

### 2H), 3.77-3.56 (m, 2H), 2.81-2.44 (m, 11H), 2.30 (t, J=7.5 Hz, 4H), 1.85-1.18 (m, 68H), 1.04 (s, 9H), 0.93-0.77 (m, 12H).

(iv) 7-((4-((tert-Butyldiphenylsilyl)oxy)butyl)amino)-1,13-bis(heptylthio)tridecane-2,12-diyl dinonanoate (101) Obtained from ketone 100 and OTBDPS-protected 4-amino-1-

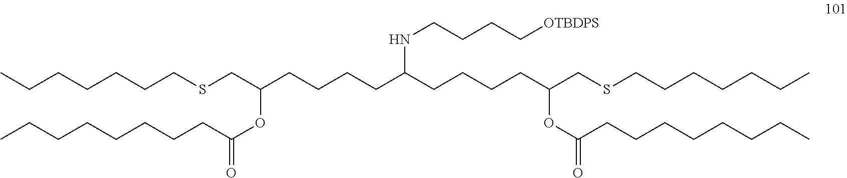

butanol 40. ¹H NMR (400 MHz, CDCl₃) δ 7.68-7.62 (m, 4H), 7.45-7.34 (m, 6H), 5.00-4.88 (m, 2H), 3.69-3.62 (m, 2H), 2.73-2.38 (m, 11H), 2.30 (t, J=7.5 Hz, 4H), 1.86-1.17 (m, 64H), 1.04 (s, 9H), 0.91-0.81 (m, 12H).

(v) 8-((4-((tert-Butyldiphenylsilyl)oxy)butyl)amino)-1,15-bis(pentylthio)pentadecane-2,14-diyl dinonanoate (105). Obtained from ketone 104 and OTBDPS-protected 4-amino-1-butanol 40. ¹H NMR (400 MHz, CDCl₃) δ 7.75-7.60 (m, 4H),

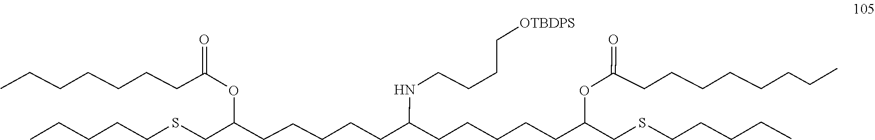

### 7.49-7.34 (m, 6H), 5.01-4.91 (m, 2H), 3.72-3.66 (m, 2) 2.79-2.46 (m, 11H), 2.32 (t, J=7.5 Hz, 4H), 1.79-1.18 (m, 60H), 1.06 (s, 9H), 0.95-0.80 (m, 12H).

(vi) 8-((4-((tert-Butyldiphenylsilyl)oxy)butyl)amino)-1,15-bis(pentylthio)pentadecane-2,14-diyl bis(decanoate) (108). Obtained from ketone 107 and OTBDPS-protected 4-amino-1-butanol 40. ¹H NMR (400 MHz, CDCl₃) δ 7.72-7.61 (m, 4H),

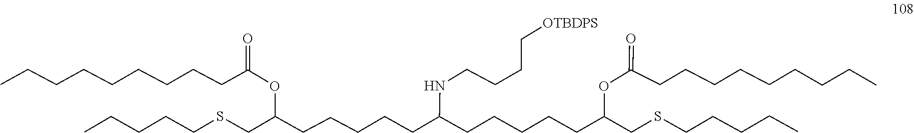

### 7.46-7.35 (m, 6H), 5.02-4.86 (m, 2H), 3.72-3.59 (m, 2H), 2.89-2.39 (m, 11H), 2.29 (t, J=7.5 Hz, 4H), 1.93-1.18 (m, 64H), 1.04 (s, 9H), 0.97-0.76 (m, 12H).

(vii) 8-((4-((tert-Butyldiphenylsilyl)oxy)butyl)amino)-1,15-bis(hexylthio)pentadecane-2,14-diyl bis(decanoate) (110). Obtained from ketone 69 and

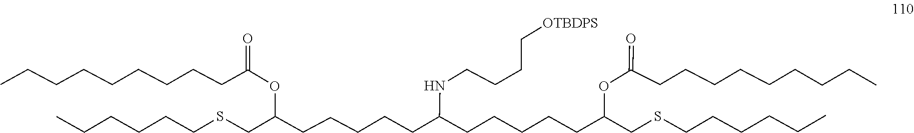

OTBDPS-protected 4-amino-1-butanol 40. ¹H NMR (400 MHz, CDCl₃) δ 7.73-7.60 (m, 4H), 7.45-7.33 (m, 6H), 5.01-4.84 (m, 2H), 3.67 (t, J=5.6 Hz, 2H), 2.74-2.44 (m, 11H), 2.29 (t, J=7.5 Hz, 4H), 1.84-1.18 (m, 68H), 1.04 (s, 9H), 0.95-0.76 (m, 12H).

(viii) 10-(6-((N-Hexanoyl-N-methylglycyl)oxy)-7-(hexylthio)heptyl)-2,2-dimethyl-3,3-diphenyl-4-oxa-18-thia-9-aza-3-silatetracosan-16-yl decanoate (114). Obtained from ketone 76 and OTBDPS-protected 4-amino-1-butanol 40. ¹H NMR (400 MHz, CDCl₃, rotamers)

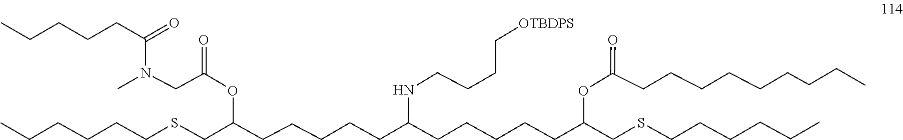

δ 7.66 (m, 4H), 7.47-7.33 (m, 6H), 5.10-4.88 (m, 2H), 4.26-4.01 (m, 2H), 3.66 (tr, J=5.8, 2H), 3.06 and 2.98 (s, 3H), 2.68-2.60 (m, 4H), 2.58-2.50 (m, 6H), 2.43 (br, 1H), 2.36 (tr, J=7.7, 2H), 2.33-2.22 (m, 3H), 1.77-1.48 (m, 16H), 1.41-1.21 (m, 44H), 1.04 (s, 9H), 0.91-0.85 (tr, 12H).

(ix) 8-((4-((tert-Butyldiphenylsilyl)oxy)butyl)amino)-1,15-bis(heptylthio)pentadecane-2,14-diyl dinonanoate (118). Obtained from ketone 117 and OTBDPS-protected 4-amino-1-butanol 40. ¹H NMR (400 MHz, CDCl₃) δ 7.76-7.62 (m, 4H),

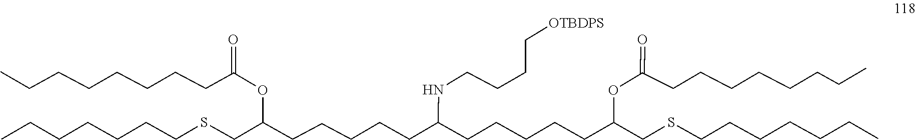

### 7.46-7.34 (m, 6H), 5.01-4.87 (m, 2H), 3.70-2.75-2.43 (m, 11H), 2.29 (t, J=7.5 Hz, 4H), 1.79-1.17 (m, 68H), 1.04 (s, 9H), 0.91-0.83 (m, 12H).

(x) 8-((4-((tert-Butyldiphenylsilyl)oxy)butyl)amino)-1,15-bis(cyclohexylthio)pentadecane-2,14-diyl dinonanoate (120). Obtained from ketone 81 and OTBDPS-protected 4-amino-1-butanol 40. ¹H NMR (400 MHz, CDCl₃ δ 7.76-7.65 (m, 4H),

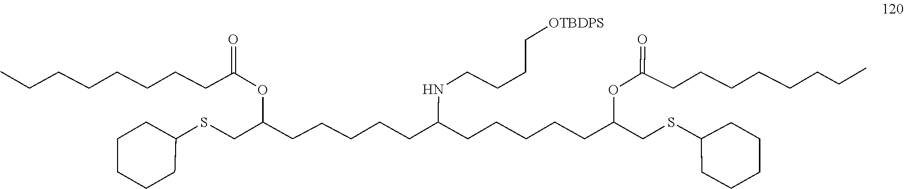

### 7.59-7.36 (m, 6H), 4.99-4.86 (m, 2H), 3.69 (t, J=6.0 Hz, 2H), 2.97-2.47 (m, 9H), 2.32 (t, J=7.5 Hz, 4H), 2.01-1.88 (m, 4H), 1.83-1.16 (m, 58H), 1.06 (s, 9H), 0.96-0.79 (m, 12H).

(xi) General procedure for reductive methylation of secondary amines. A solution of a secondary amine (1 mmol), aq. formaldehyde (37%, 6 mL) and NaBH(OAc)₃ (5 mmol) in THF (10 mL) was stirred under inert atmosphere at room temperature for 3 days. The reaction was then quenched with sat. aq. NaHCO₃ (10 mL), diluted with water (10 mL) and extracted with CH₂Cl₂ (3×15 mL). The combined extracts were dried (Na₂SO₄) and concentrated. The residue was purified by silica chromatography (0-5% MeOH in DCM) to yield the tertiary methylamine (75-85%) as an oil.

(xii) General procedure for reductive alkylation of secondary amines. To a solution of a secondary amine (1 mmol) and an aldehyde (5 mmol) in DCE (15 mL) was added NaBH(OAc)₃ (5 mmol) and HOAc (0.2 mL). The resulting mixture was stirred under inert atmosphere at room temperature for 18 hours then quenched with sat. aq. NaHCO₃ (5 mL), diluted with water (15 mL) and extracted with DCM (3×15 mL). The combined extracts were dried (Na₂SO₄) and concentrated. The residue was purified by silica chromatography (0-5% MeOH in DCM) to yield the tertiary amine (70-80%) as an oil.

(xiii) 7-((4-((tert-Butyldiphenylsilyl)oxy)butyl)(methyl)amino)-1,13-bis(pentylthio)tridecane-2,12-diyl bis(decanoate) (95). Obtained by procedure (xi) from

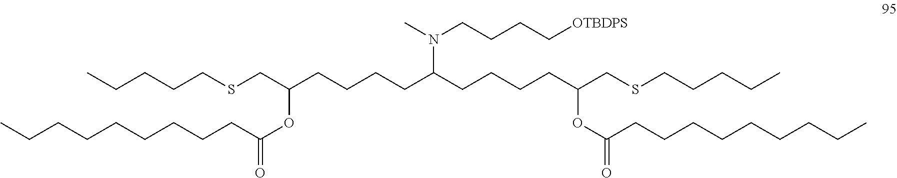

secondary amine 94. ¹H NMR (400 MHz, CDCl₃) δ 7.74-7.56 (m, 4H), 7.49-7.31 (m, 6H), 5.05-4.91 (m, 2H), 3.66 (t, J=6.2 Hz, 2H), 2.66-2.60 (m, 4H), 2.53 (td, J=7.3, 1.7 Hz, 4H), 2.39-2.23 (m, 7H), 2.11 (s, 3H), 1.74-1.12 (m, 60H), 1.04 (s, 9H), 0.93-0.83 (m, 12H).

(xiv) 7-((4-((tert-Butyldiphenylsilyl)oxy)butyl)(methyl)amino)-1,13-bis(heptylthio)tridecane-2,12-diyl bis(decanoate) (99). Obtained by procedure (xi) from

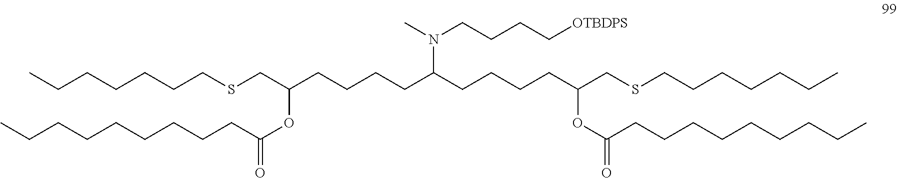

secondary amine 98. ¹H NMR (400 MHz, CDCl₃) δ 7.73-7.62 (m, 4H), 7.47-7.36 (m, 6H), 5.03-4.93 (m, 2H), 3.68 (t, J=6.1 Hz, 2H), 2.75-2.62 (m, 4H), 2.55 (td, J=7.3, 1.6 Hz, 4H), 2.42-2.26 (m, 7H), 2.14 (s, 3H), 1.78-1.14 (m, 68H), 1.07 (s, 9H), 0.99-0.83 (m, 12H).

(xv) 7-((4-((tert-Butyldiphenylsilyl)oxy)butyl)(methyl)amino)-1,13-bis(heptylthio)tridecane-2,12-diyl dinonanoate (102). Obtained by procedure (xi) from

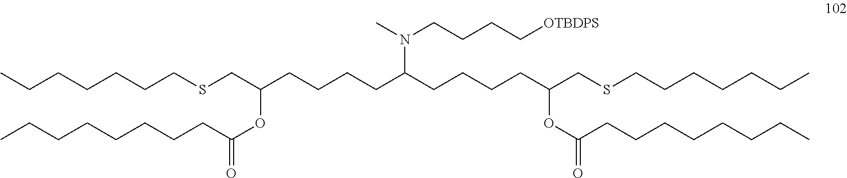

secondary amine 101. ¹H NMR (400 MHz, CDCl₃) δ 7.80-7.59 (m, 4H), 7.52-7.37 (m, 6H), 5.06-4.90 (m, 2H), 3.68 (t, J=6.2 Hz, 2H), 2.72-2.62 (m, 4H), 2.55 (td, J=7.3, 1.6 Hz, 4H), 2.40-2.27 (m, 7H), 2.14 (s, 3H), 1.80-1.15 (m, 64H), 1.07 (s, 9H), 0.95-0.87 (m, 12H).

(xvi) 8-((4-((tert-Butyldiphenylsilyl)oxy)butyl)(methyl)amino)-1,15-bis(pentylthio)penta-decane-2,14-diyl dinonanoate (106). Obtained by procedure (xi) from

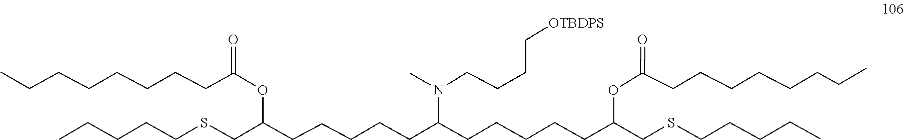

secondary amine 105. ¹H NMR (400 MHz, CDCl₃) δ 7.79-7.60 (m, 4H), 7.52-7.32 (m, 6H), 5.01-4.91 (m, 2H), 3.66 (t, J=6.1 Hz, 2H), 2.75-2.60 (m, 4H), 2.53 (td, J=7.2, 1.7 Hz, 4H), 2.40-2.23 (m, 7H), 2.12 (s, 3H), 1.77-1.13 (m, 60H), 1.04 (s, 9H), 0.99-0.82 (m, 12H).

(xvii) 8-((4-((tert-Butyldiphenylsilyl)oxy)butyl)(methyl)amino)-1,15-bis(pentylthio)penta-decane-2,14-diyl bis(decanoate) (109). Obtained by procedure (xi) from

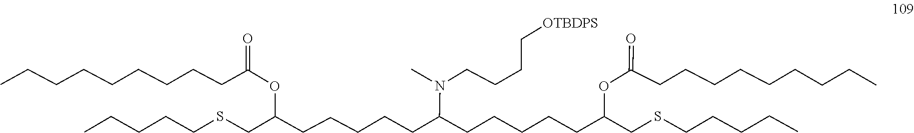

secondary amine 108. ¹H NMR (400 MHz, CDCl₃) δ 7.77-7.60 (m, 4H), 7.47-7.32 (m, 6H), 5.03-4.90 (m, 2H), 3.66 (t, J=6.1 Hz, 2H), 2.71-2.60 (m, 4H), 2.53 (td, J=7.3, 1.7 Hz, 4H), 2.44-2.22 (m, 7H), 2.12 (s, 9H), 1.83-1.16 (m, 64H), 1.04 (s, 3H), 0.98-0.77 (m, 12H).

(xviii) 8-((4-((tert-Butyldiphenylsilyl)oxy)butyl)(methyl)amino)-1,15-bis(hexylthio)penta-decane-2,14-diyl bis(decanoate) (111). Obtained by procedure (xi) from secondary amine 110. ¹H NMR (400 MHz, CDCl₃) δ 7.73-7.60 (m, 4H), 7.45-7.33 (m, 6H), 5.01-

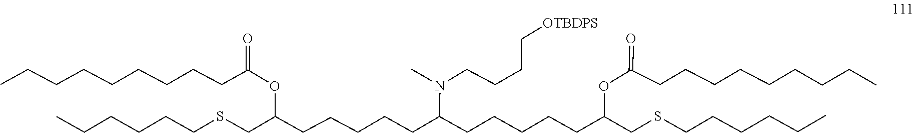

### 4.84 (m, 2H), 3.67 (t, J=5.6 Hz, 2H), 2.74-2.44 (m, 11H), 2.29 (t, J=7.5 Hz, 4H), 1.84-1.18 (m, 68H), 1.04 (s, 9H), 0.95-0.76 (m, 12H).

(xix) 8-((4-((tert-Butyldiphenylsilyl)oxy)butyl)(propyl)amino)-1,15-bis(hexylthio)pentadec-ane-2,14-diyl bis(decanoate) (112). Obtained by procedure (xii) from secondary amine 110 and ethanal (acetaldehyde). H NMR (400 MHz, CDCl₃) δ 7.74-7.61 (m,

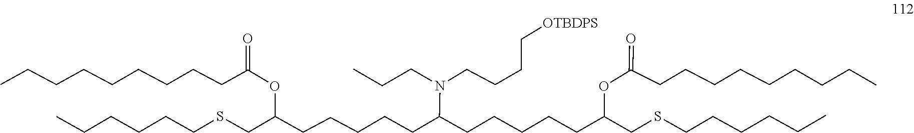

### 4H), 7.48-7.32 (m, 6H), 5.02-4.87 (m, 2H), 3.65 (t, J=6.3 Hz, 2H), 2.67-2.60 (m, 4H), 2.53 (td, J=7.3, 1.6 Hz, 4H), 2.38-2.21 (m, 9H), 1.80-1.10 (m, 70H), 1.04 (s, 9H), 0.88 (td, J=6.9, 2.6 Hz, 12H), 0.83 (t, J=7.4 Hz, 3H).

(xx) 8-((4-((tert-butyldiphenylsilyl)oxy)butyl)(isobutyl)amino)-1,15-bis(hexylthio)penta-decane-2,14-diyl bis(decanoate) (113). Obtained by procedure (xii) from secondary amine 110 and 2-methyl-propanal (isobutyraldehyde). ¹H NMR (400 MHz, CDCl₃) δ

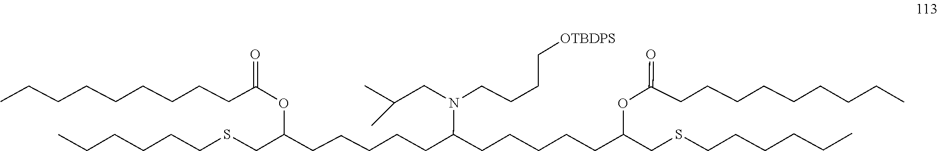

7.71-7.61 (m, 4H), 7.48-7.33 (m, 6H), 5.01-4.90 (m, 2H), 3.65 (t, J=6.3 Hz, 2H), 2.70-2.60 (m, 4H), 2.53 (td, J=7.2, 1.6 Hz, 4H), 2.28 (q, J=7.2 Hz, 7H), 2.06 (d, J=7.1 Hz, 2H), 1.86-1.09 (m, 69H), 1.04 (s, 9H), 0.91-0.85 (m, 12H), 0.82 (d, J=6.5 Hz, 6H).

(xxi) 10-(6-((N-hexanoyl-N-methylglycyl)oxy)-7-(hexylthio)heptyl)-2,2,9-trimethyl-3,3-diphenyl-4-oxa-18-thia-9-aza-3-silatetracosan-16-yl decanoate (115). Obtained by procedure (xi) from secondary amine 114. ¹H NMR (400 MHz, CDCl₃, rotamers) δ 7.66 (m, 4H), 7.44-7.34 (m, 6H), 5.11-4.87 (m, 2H), 4.25-4.00 (m, 2H), 3.66 (tr, J=5.8, 2H), 3.06 and 2.98 (s,

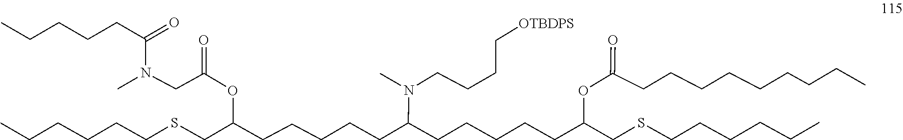

### 3H), 2.68-2.59 (m, 4H), 2.56-2.49 (m, 4H), 2.41-2.21 (m, 7H), 2.15 (s, 3H), 1.71-1.46 (m, 17H), 1.40-1.20 (m, 43H), 1.04 (s, 9H), 0.92-0.83 (tr, 12H).

(xxii) 8-((4-((tert-Butyldiphenylsilyl)oxy)butyl)(methyl)amino)-1,15-bis(heptylthio)penta-decane-2,14-diyl dinonanoate (119). Obtained by procedure (xi) from

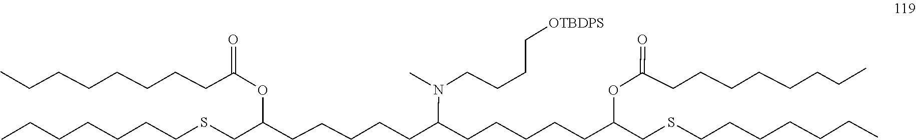

secondary amine 118. ¹H NMR (400 MHz, CDCl₃) δ 7.72-7.58 (m, 4H), 7.48-7.33 (m, 6H), 5.03-4.90 (m, 2H), 3.66 (t, J=6.1 Hz, 2H), 2.63 (d, J=6.0 Hz, 4H), 2.53 (td, J=7.3, 1.7 Hz, 4H), 2.31 (dt, J=15.0, 7.6 Hz, 7H), 2.12 (s, 3H), 1.80-1.10 (m, 68H), 1.04 (s, 9H), 0.98-0.82 (m, 12H).

(xxiii) 8-((4-((tert-Butyldiphenylsilyl)oxy)butyl)(methyl)amino)-1,15-bis(cyclohexylthio)-pentadecane-2,14-diyl dinonanoate (121). Obtained by procedure (xi)

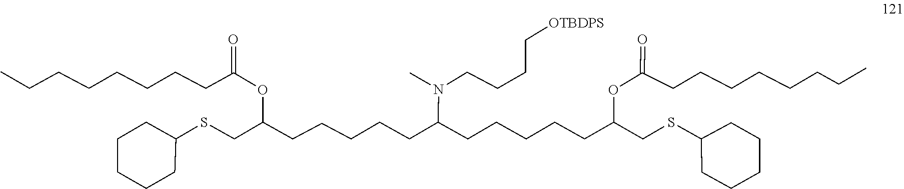

from secondary amine 120. ¹H NMR (400 MHz, CDCl₃) δ 7.73-7.61 (m, 4H), 7.50-7.35 (m, 6H), 5.06-4.86 (m, 2H), 3.66 (t, J=6.1 Hz, 2H), 2.79-2.59 (m, 6H), 2.47-2.23 (m, 7H), 2.15 (s, 3H), 2.03-1.93 (m, 4H), 1.82-1.13 (m, 64H), 1.04 (s, 9H), 0.96-0.81 (m, 6H).

(H) General procedure for release of silyl protecting groups: synthesis of lipids 5 and 22-35. To a solution of a TBDPS-protected compound (1 mmol) in THF (5 mL) was added HF-pyridine (1 mL) at 0° C. under inert atmosphere. The reaction was warmed to room temperature and stirred for 18 hours. Water (10 mL) was added, and the mixture was extracted with DCM (3×15 mL). The combined organics were dried (Na₂SO₄) and concentrated. The residue was purified by silica chromatography (0-5% MeOH in DCM) to yield the corresponding lipid (55-70%) as an oil. The following lipids were thus obtained:

(i) ((4-Hydroxybutyl)azanediyl)bis(1-(pentylthio)heptane-7,2-diyl)bis(decanoate)

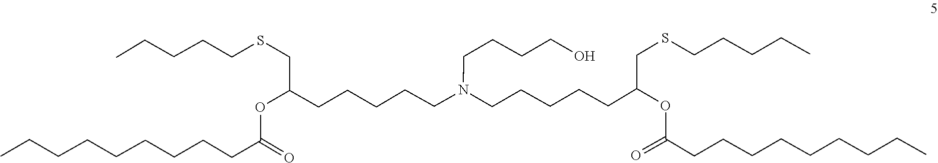 [SMILES: CCCCCCCCCC(=O)OC(CCCCCN(CCCCO)CCCCCC(CSCCCCC)OC(=O)CCCCCCCCC)CSCCCCC]

(5). Obtained from compound 46. ¹H NMR (400 MHz, CDCl₃) δ 5.00-4.89 (m, 2H), 3.62 (t, J=4.9 Hz, 2H), 2.78-2.57 (m, 10H), 2.53 (td, J=7.3, 1.5 Hz, 4H), 2.30 (t, J=7.6 Hz, 4H), 1.83-1.49 (m, 20H), 1.43-1.17 (m, 40H), 0.96-0.82 (m, 12H).

(ii) 7-((4-Hydroxybutyl)(methyl)amino)-1,13-bis(pentylthio)tridecane-2,12-diyl bis(decano-ate) (22). Obtained from compound 95. ¹H NMR (400 MHz, CDCl₃) δ 5.06-4.88 (m,

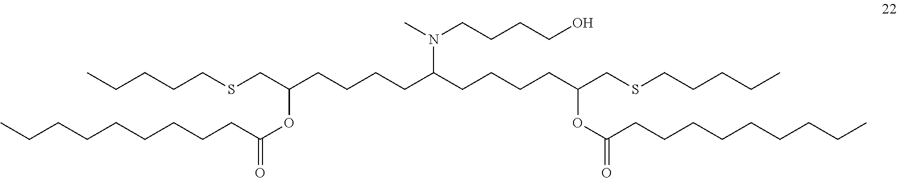 [SMILES: CCCCCCCCCC(=O)OC(CCCCC(CCCCC(CSCCCCC)OC(=O)CCCCCCCCC)N(C)CCCCO)CSCCCCC]

### 2H), 3.73-3.58 (m, 2H), 2.76-2.61 (m, 7H), 2.53 (td, J=7.3, 1.4 Hz, 4H), 2.39-2.26 (m, 7H), 1.81-1.20 (m, 60H), 0.94-0.81 (m, 12H).

(iii) 1,13-bis(Heptylthio)-7-((4-hydroxybutyl)(methyl)amino)tridecane-2,12-diyl bis(decano-ate) (23). Obtained from compound 99. ¹H NMR (400 MHz, CDCl₃) δ 5.01-4.89 (m,

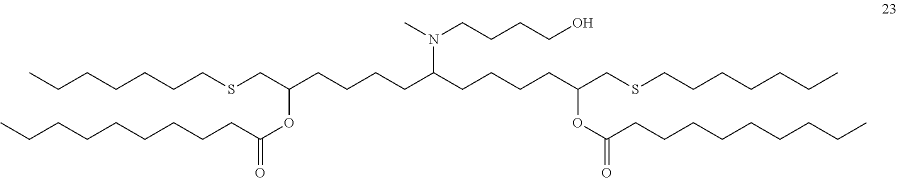 [SMILES: CCCCCCCCCC(=O)OC(CCCCC(CCCCC(CSCCCCCCC)OC(=O)CCCCCCCCC)N(C)CCCCO)CSCCCCCCC]

### 2H), 3.61 (t, J=4.9 Hz, 2H), 2.69-2.57 (m, 7H), 2.53 (td, J=7.2, 1.4 Hz, 4H), 2.36-2.25 (m, 7H), 1.79-1.18 (m, 68H), 0.92-0.85 (m, 12H).

(iv) 1,13-bis(Heptylthio)-7-((4-hydroxybutyl)(methyl)amino)tridecane-2,12-diyl dinonano-ate (24). Obtained from compound 102. H NMR (400 MHz, CDCl₃) δ 5.01-4.88 (m,

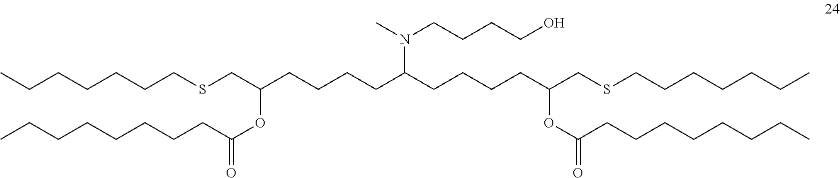 [SMILES: CCCCCCCCC(=O)OC(CCCCC(CCCCC(CSCCCCCCC)OC(=O)CCCCCCCC)N(C)CCCCO)CSCCCCCCC]

### 2H), 3.65-3.52 (m, 2H), 2.75-2.60 (m, 4H), 2.59-2.38 (m, 7H), 2.30 (t, J=7.5 Hz, 4H), 2.16 (s, 3H), 1.82-1.12 (m, 64H), 0.96-0.83 (m, 12H).

(v) 8-((4-hydroxybutyl)(methyl)amino)-1,15-bis(pentylthio)pentadecane-2,14-diyl dinonano-ate (25). Obtained from compound 106. H NMR (400 MHz, CDCl₃) δ 5.03-4.88 (m,

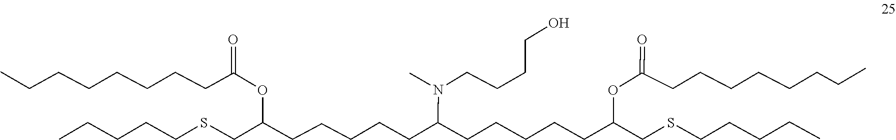 [SMILES: CCCCCCCCC(=O)OC(CCCCCC(CCCCCC(CSCCCCC)OC(=O)CCCCCCCC)N(C)CCCCO)CSCCCCC]

### 2H), 3.67-3.55 (m, 2H), 2.69-2.62 (m, 4H), 2.60-2.49 (m, 7H), 2.30 (t, J=7.5 Hz, 4H), 2.24 (s, 3H), 1.78-1.20 (m, 60H), 0.93-0.84 (m, 12H).

(vi) 8-((4-hydroxybutyl)(methyl)amino)-1,15-bis(pentylthio)pentadecane-2,14-diyl bis-(decanoate) (26). Obtained from compound 109. ¹H NMR (400 MHz, CDCl₃) δ 5.03-4.85

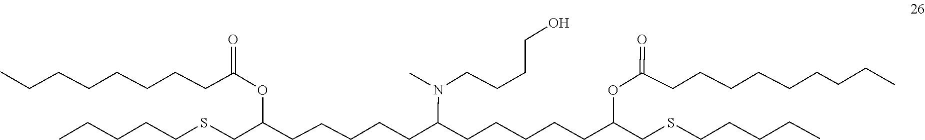 [SMILES: CCCCCCCCCC(=O)OC(CCCCCC(CCCCCC(CSCCCCC)OC(=O)CCCCCCCC)N(C)CCCCO)CSCCCCC]

### (m, 2H), 3.65-3.52 (m, 2H), 2.72-2.59 (m 4H), 2.53 (td, J=7.3, 1.6 Hz, 7H), 2.30 (t, J=7.5 Hz, 4H), 2.18 (s, 3H), 1.79-1.15 (m, 64H), 0.95-0.84 (m, 12H).

(vii) 1,15-bis(Hexylthio)-8-((4-hydroxybutyl)(methyl)amino)pentadecane-2,14-diyl bis-(decanoate) (27). Obtained from compound 111. ¹H NMR (400 MHz, CDCl₃) δ 5.01-4.88

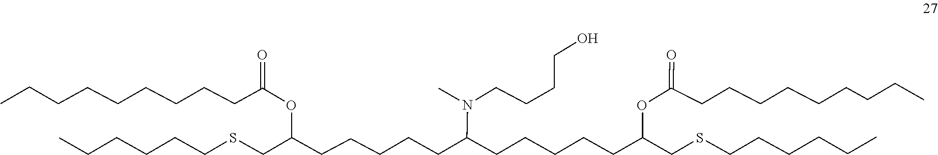 [SMILES: CCCCCCCCCC(=O)OC(CCCCCC(CCCCCC(CSCCCCCC)OC(=O)CCCCCCCCC)N(C)CCCCO)CSCCCCCC]

### (m, 2H), 3.65-3.55 (m, 2H), 2.70-2.48 (m, 11H), 2.36-2.20 (m, 7H), 1.81-1.22 (m, 68H), 0.96-0.84 (m, 12H).

(viii) 1,15-bis(hexylthio)-8-((4-hydroxybutyl)(propyl)amino)pentadecane-2,14-diyl bis-(decanoate) (28). Obtained from compound 112. ¹H NMR (400 MHz, CDCl₃) δ 5.44-5.33

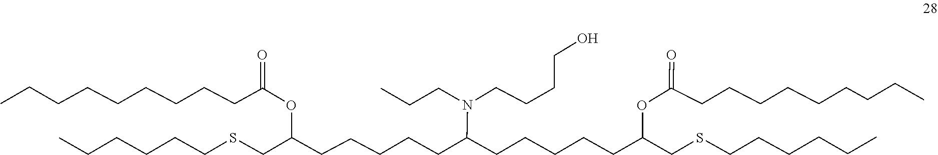 [SMILES: CCCCCCCCCC(=O)OC(CCCCCC(CCCCCC(CSCCCCCC)OC(=O)CCCCCCCCC)N(CCC)CCCCO)CSCCCCCC]

### (m, 2H), 3.73-3.64 (m, 2H), 2.88-2.72 (m, 5H), 2.71-2.52 (m, 6H), 2.51-2.44 (m, 2H), 2.40 (t, J=7.4 Hz, 4H), 1.99-1.20 (m, 70H), 1.07-0.94 (m, 15H).

(ix) 1,15-bis(Hexylthio)-8-((4-hydroxybutyl)(isobutyl)amino)pentadecane-2,14-diyl bis-(decanoate) (29). Obtained from compound 113. ¹H NMR (400 MHz, C₆D₆) δ 5.33-5.21 (m,

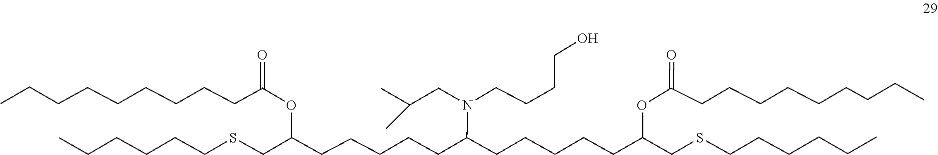 [SMILES: CCCCCCCCCC(=O)OC(CCCCCC(CCCCCC(CSCCCCCC)OC(=O)CCCCCCCCC)N(CCCCO)CC(C)C)CSCCCCCC]

### 2H), 3.54-3.48 (m, 2H), 2.67 (qd, J=13.6, 6.2 Hz, 4H), 2.59-2.44 (m, 5H), 2.42-2.33 (m, 2H), 2.33-2.24 (m, 4H), 2.16 (d, J=7.1 Hz, 2H), 1.87-1.11 (m, 69H), 1.02-0.81 (m, 18H).

(x) 14-((N-hexanoyl-N-methylglycyl)oxy)-1,15-bis(hexylthio)-8-((4-hydroxybutyl)(methyl)-amino)pentadecan-2-yl decanoate (30). Obtained from compound 115. ¹H NMR (400 MHz, CDCl₃, rotamers) δ 5.02 (m, 1H), 4.94 (m, 1H), 4.32-3.87 (m, 2H),

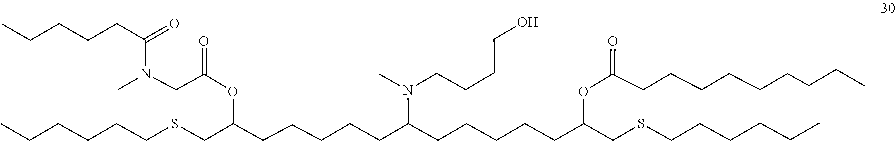 [SMILES: CCCCCCCCCC(=O)OC(CCCCCC(CCCCCC(CSCCCCCC)OC(=O)CN(C)C(=O)CCCCC)N(C)CCCCO)CSCCCCCC]

### 3.77 (m, 1H), 3.67 (m, 1H), 3.25-2.94 (6H), 2.84-2.72 (m, 3H), 2.64 (d, J=6 Hz, 4H), 2.58-2.50 (m, 4H), 2.37 (m, 2H), 2.31 (m, 2H), 2.00 (1H), 1.87 (2H), 1.79-1.50 (m, 16H), 1.47-1.21 (m, 40H), 0.94-0.81 (tr, 12H).

(xi) 1,15-bis(Heptylthio)-8-((4-hydroxybutyl)(methyl)amino)pentadecane-2,14-diyl dinonanoate (31). Obtained from compound 119. ¹H NMR (400 MHz, CDCl₃) δ 5.02-4.89 (m,

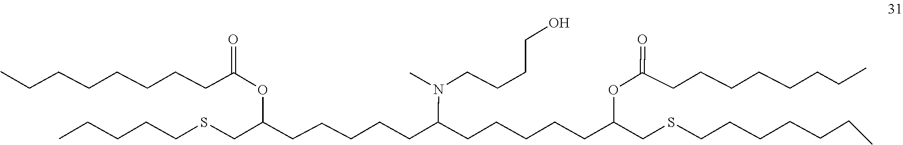 [SMILES: CCCCCCCCC(=O)OC(CCCCCC(CCCCCC(CSCCCCCCC)OC(=O)CCCCCCCC)N(C)CCCCO)CSCCCCC]

### 2H), 3.68-3.54 (m, 2H), 2.69-2.60 (m, 4H), 2.58-2.46 (m, 7H), 2.30 (t, J=7.5 Hz, 4H), 2.21 (s, 3H), 1.78-1.20 (m, 68H), 0.96-0.82 (m, 12H).

(xii) 1,15-bis(Cyclohexylthio)-8-((4-hydroxybutyl)(methyl)amino)pentadecane-2,14-diyl dinonanoate (32). Obtained from compound 121 and characterized as the HCl salt. ¹H NMR (400 MHz, CDCl₃) δ 4.97-4.86 (m, 2H), 3.73 (t, J=5.3 Hz, 2H), 3.16-3.11 (m, 1H), 3.07

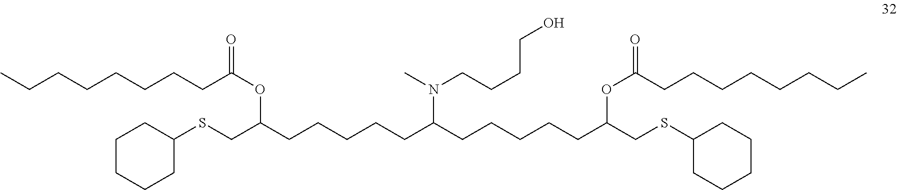 [SMILES: CCCCCCCCC(=O)OC(CCCCCC(CCCCCC(CSC1CCCCC1)OC(=O)CCCCCCCC)N(C)CCCCO)CSC1CCCCC1]

### (t, J=6.6 Hz, 2H), 2.73 (s, 3H), 2.70-2.56 (m, 6H), 2.30 (t, J=7.6 Hz, 4H), 2.07-1.13 (m, 68H), 0.93-0.73 (m, 6H).

### Example 2: MRNA-Containing LNPs Comprising Ionizable Lipids 1 or 2 Exhibit In Vivo Delivery of mRNA to the Liver and Spleen that is Superior to the MC3 Benchmark

LNP formulations containing 50/10/38.5/1.5 mol % of ionizable lipids/DSPC/chol/PEG-DMG with a nitrogen-to-phosphorous ratio (N/P) of 6 and mRNA encoding luciferase were tested for in vivo transfection efficiency in the liver and spleen after injection to CD-1 mice. The mRNA dose was 1 mg/kg. Luminescence intensity in the liver and spleen was measured at 4 hours post-injection. The ionizable lipids were 1 and 5-32 (Table 1).

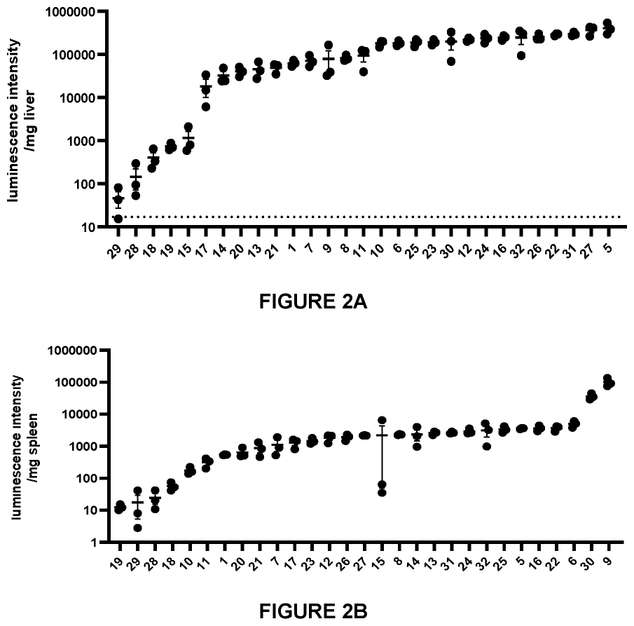

The results in FIG. 2A show that luminescence intensity per mg liver was higher for lipids 7, 9, 8, 11, 10, 6, 25, 23, 30, 12, 24, 30, 12, 24, 16, 32, 26, 22 31, 27 and 5 than the MC3 benchmark. Results for luminescence intensity per mg spleen were found to be higher for lipids 20, 21, 7, 17, 23, 12, 26, 27, 15, 8, 14, 13, 31, 24, 32, 25, 5, 16, 22, 6, 30 and 9 than the MC3 benchmark (FIG. 2B).

1. A lipid having a structure of Formula A: 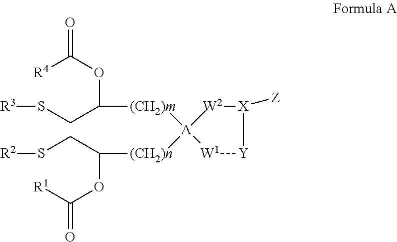
or a pharmaceutically acceptable salt thereof;
wherein
m is 4 to 8; and
n is 4 to 8;
R² and R³ are independently linear or branched optionally substituted C₃ to C₂₀ alkyl and optionally comprising 0-2 carbon-carbon double bonds, or are independently unsubstituted, saturated C₆ alkyl;
R¹ and R⁴ are independently linear or branched optionally substituted C₃ to C₂₀ alkyl and optionally comprising 0-2 carbon-carbon double bonds;
A is either C or N, and
if A is C, then W¹ and Y are either bonded to each other or not bonded to each other, and
if W¹ and Y are bonded to each other, then
W¹ is O or S;
W² is O or S;
X is CH;
Y is (CH₂)_{q}, wherein q is 1 or 2;
Z is selected from one of structures a-c below, wherein the wavy line represents the bond to X:
a. 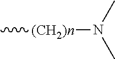
type 2 ionizable head group, wherein the n of the type 2 ionizable head group is 1 to 5;
b. 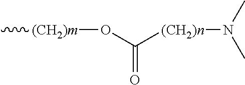
type 3 ionizable head group, wherein the m and n of the type 3 ionizable head group are independently 1 to 5;
c. 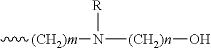
type 4 ionizable head group, wherein the m and n of the type 4 ionizable head group are independently 2 to 5 and wherein R=C₁-C₆ alkyl or cycloalkyl;
if W¹ and Y are not bonded to each other, then
W¹ is H;
W² is O, S, NH or NR^{2a}, wherein R^{2a} is a C₁ to C₄ alkyl optionally substituted with an OH group; and the moiety 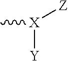
of Formula A is a group selected from structures d-h below, wherein the wavy line represents the bond to W²:
d. 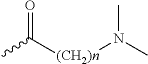
type 1 ionizable head group, wherein the n of the type 1 ionizable head group is 1 to 5;
e. 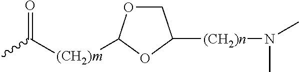
type 5 ionizable head group, wherein the m and n of the type 5 ionizable head group are independently 1 to 5;
f. 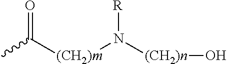
type 6 ionizable head group, wherein the m of the type 6 ionizable head group is 1 to 5 and the n is independently 2 to 5 and wherein the R=C₁-C₆ alkyl or cycloalkyl;
g. [symbol] (CH₂)ₙ—OH type 7 ionizable head group, wherein the n of the type 7 ionizable head group is 1 to 5;
h. 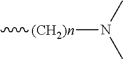
type 8 ionizable head group, wherein the n of the type 7 ionizable head group is 1 to 5;
i. 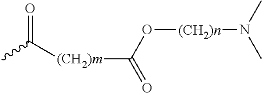
type 9 ionizable head group, wherein m and n of the type 9 ionizable head group are independently 1 to 5;
if A is N, then
W¹ and Y are absent;
W² and X together form a group of structure (CR^{a}R^{b})ₚ, wherein R^{a} and R^{b} are independently H or C₁-C₈alkyl or cycloalkyl, and wherein p is 2 to 6; and
Z is OH or NR′R″, wherein R′ and R″ are independently optionally substituted C₁-C₈alkyl or cycloalkyl, or wherein R′ and R″ together with the N atom of NR′R″, form an optionally substituted heterocyclic ring that incorporates the N atom to which the R′ and R″ are each bound.

2. The lipid or the pharmaceutically acceptable salt of claim 1, wherein at least one of R¹ and R⁴ are, independently, a moiety of Formula B, wherein: 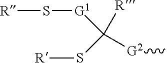
Formula B
R′ and R″ are, independently, linear or branched optionally substituted C₃ to C₁₂ alkyl groups and optionally comprising 0-2 carbon-carbon double bonds;
R′″ is H or a linear, branched, or cyclic optionally substituted C₁ to C₆ alkyl group; and
G¹ and G² are, independently, (CR^{a}R^{b})ₚ, wherein R^{a} and R^{b} are each independently selected from H or optionally substituted C₁-C₅ alkyl or cycloalkyl, wherein p is 0 to 6.

3. The lipid or the pharmaceutically acceptable salt of claim 1, wherein A is N, W¹ and Y are absent, W² and X together form a group of the structure (CR^{a}R^{b})ₚ, and Z is NR′R″ and wherein the heterocyclic group that incorporates the N atom to which R′ and R″ are bound is pyrrolidine, piperidine or morpholine.

4. The lipid or the pharmaceutically acceptable salt of claim 1, wherein A is a carbon atom.

5. The lipid or the pharmaceutically acceptable salt of claim 4, wherein W¹ and Y are not bonded to each other.

6. The lipid or the pharmaceutically acceptable salt of claim 5, wherein W² is O.

7. The lipid or the pharmaceutically acceptable salt of claim 6, wherein the moiety 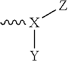
of Formula A is structure d.

8. The lipid or the pharmaceutically acceptable salt of claim 1 having a structure of any one of the following compounds 5-14, 16, 19, 20 and 22-32:

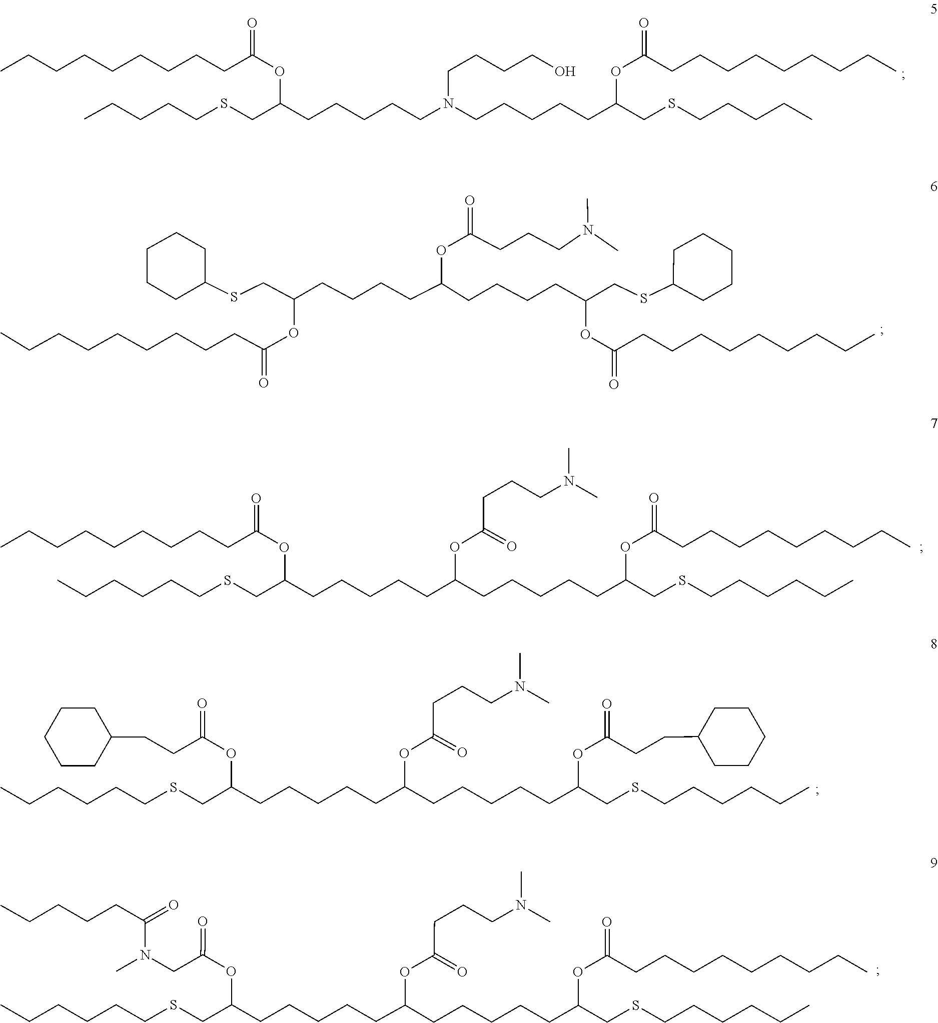

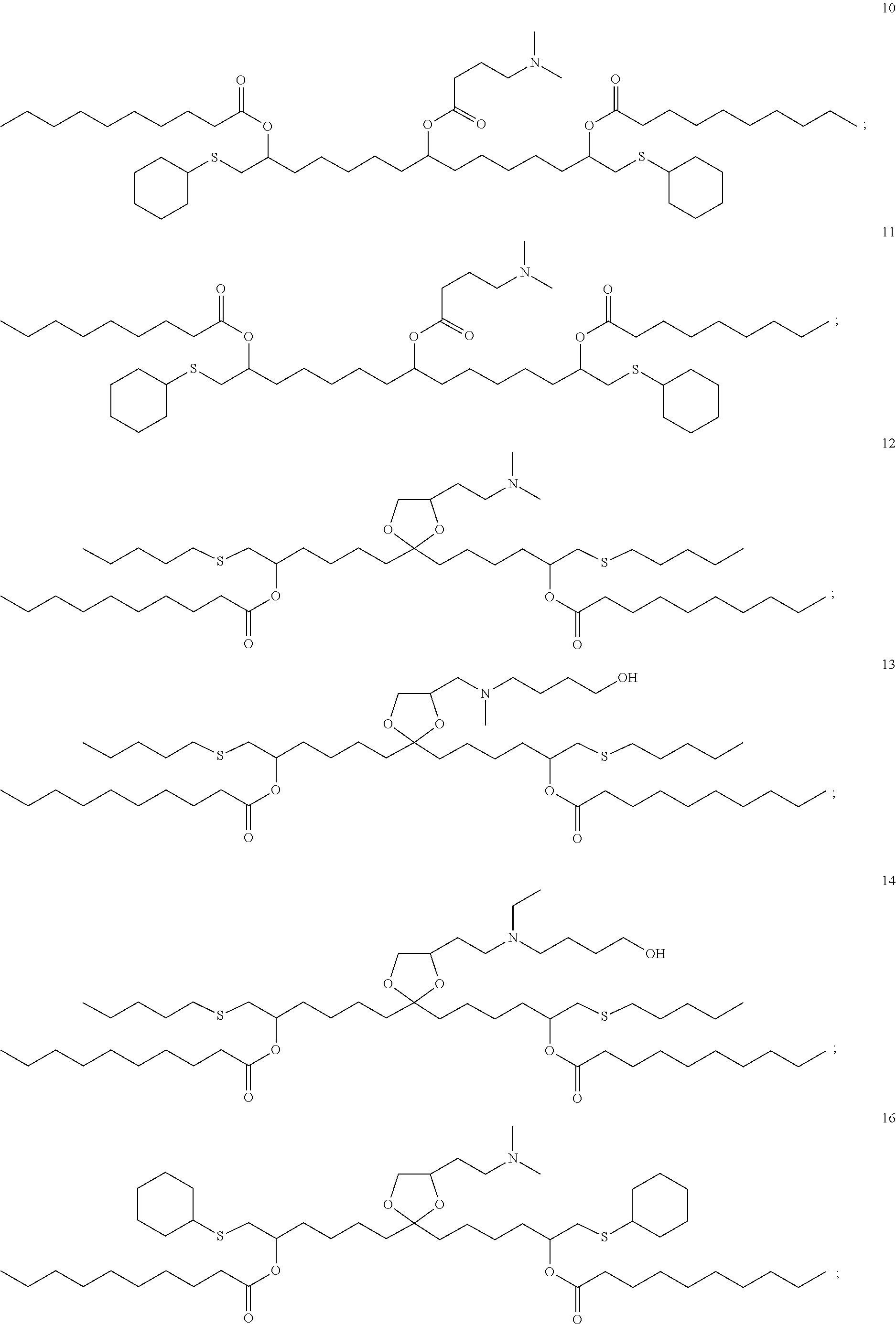

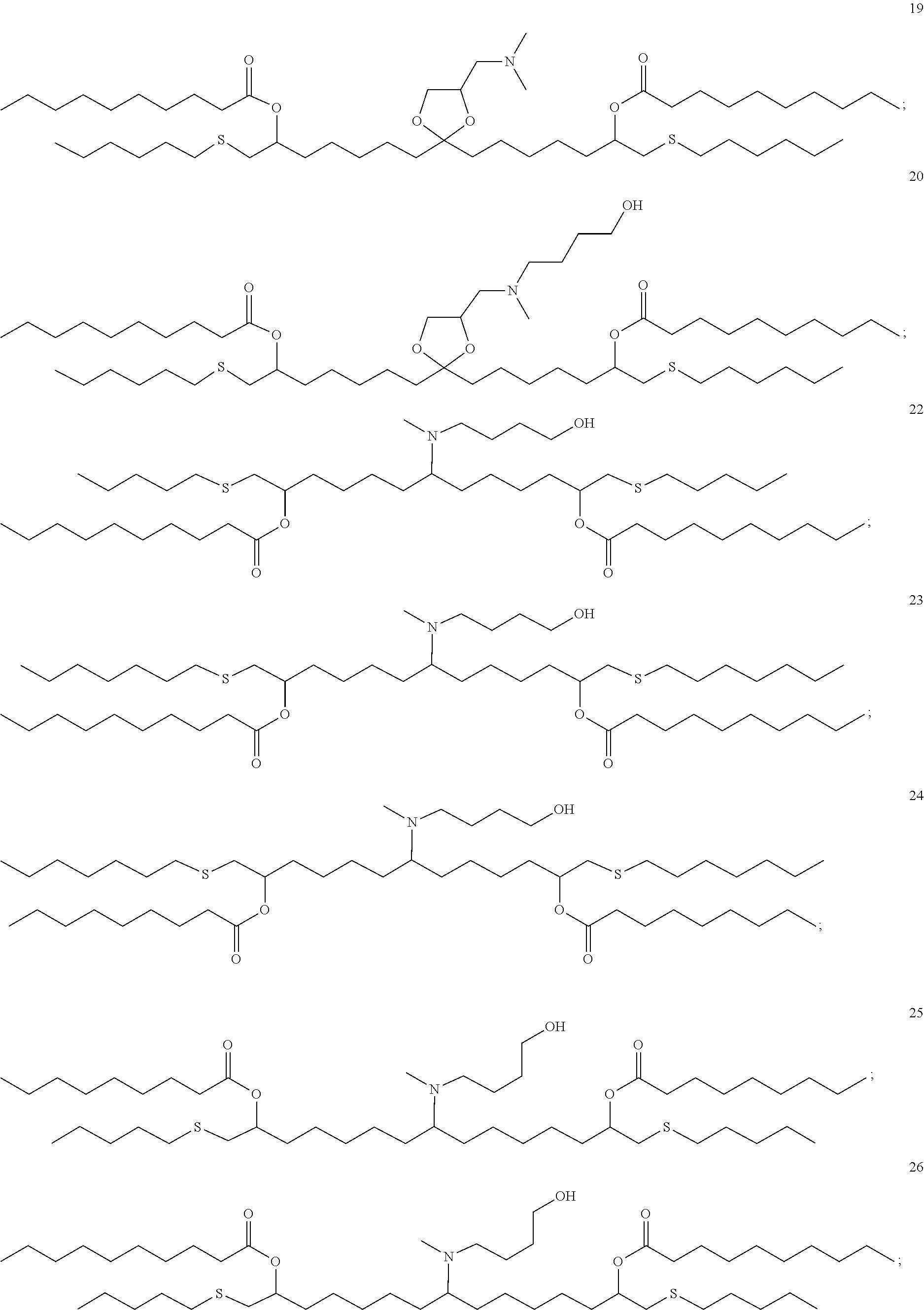

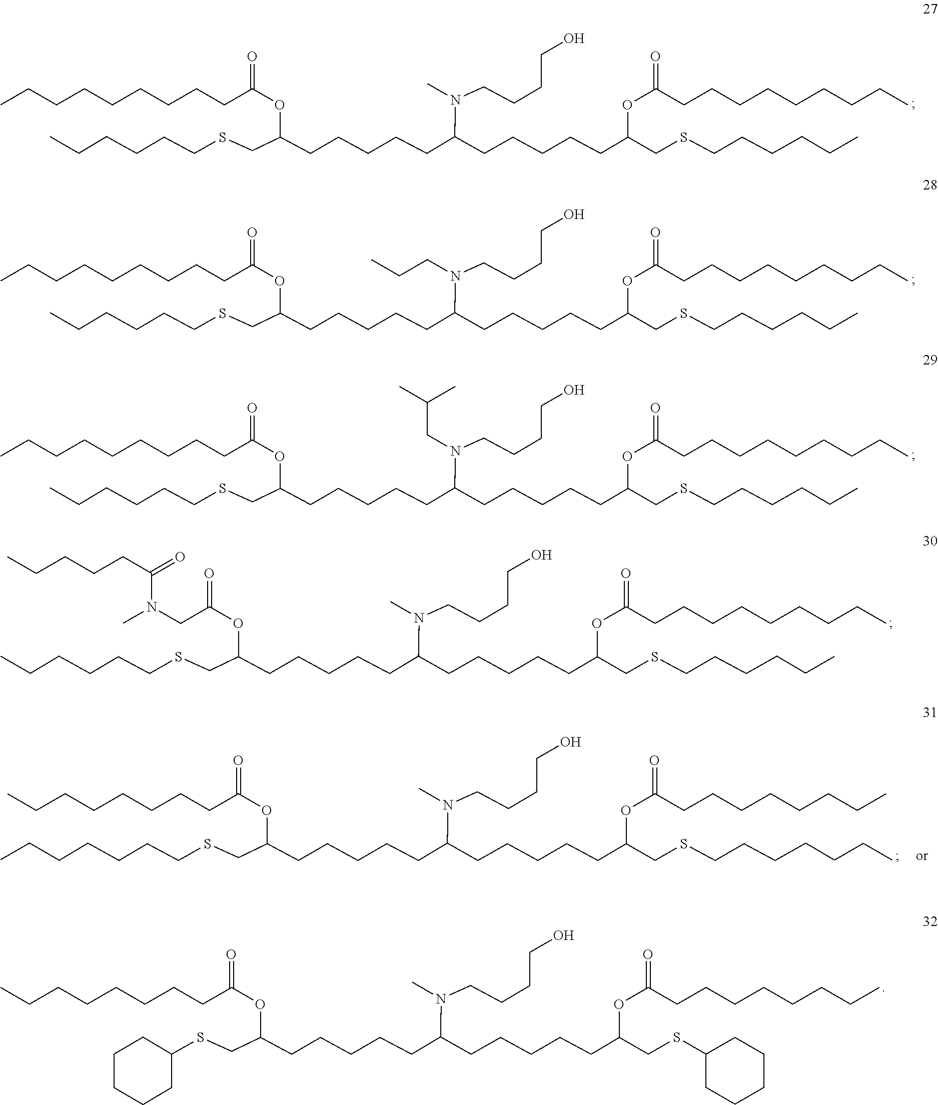

9. A lipid or a pharmaceutically acceptable salt thereof comprising:
a protonatable amino head group;
two lipophilic chains, wherein the amino head group has a central nitrogen atom or carbon atom to which each of the two lipophilic chains are directly bonded;
at least one of the lipophilic chains has the formula: 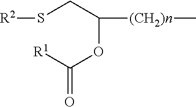
wherein R¹ is a linear or branched optionally substituted C₃-C₂₀ alkyl and optionally with varying degrees of unsaturation;
wherein R² is a linear or branched optionally substituted C₃-C₂₀ alkyl and optionally with varying degrees of unsaturation or is an unsubstituted, saturated C₆ alkyl;
n is 4 to 8;
each lipophilic chain has between 15 and 40 carbon atoms in total; and
wherein the lipid is ionizable and has a Clog P of at least 11.

10. The lipid or the pharmaceutically acceptable salt of claim 9 having a structure of any one of the following compounds 15, 17, 18 and 21:

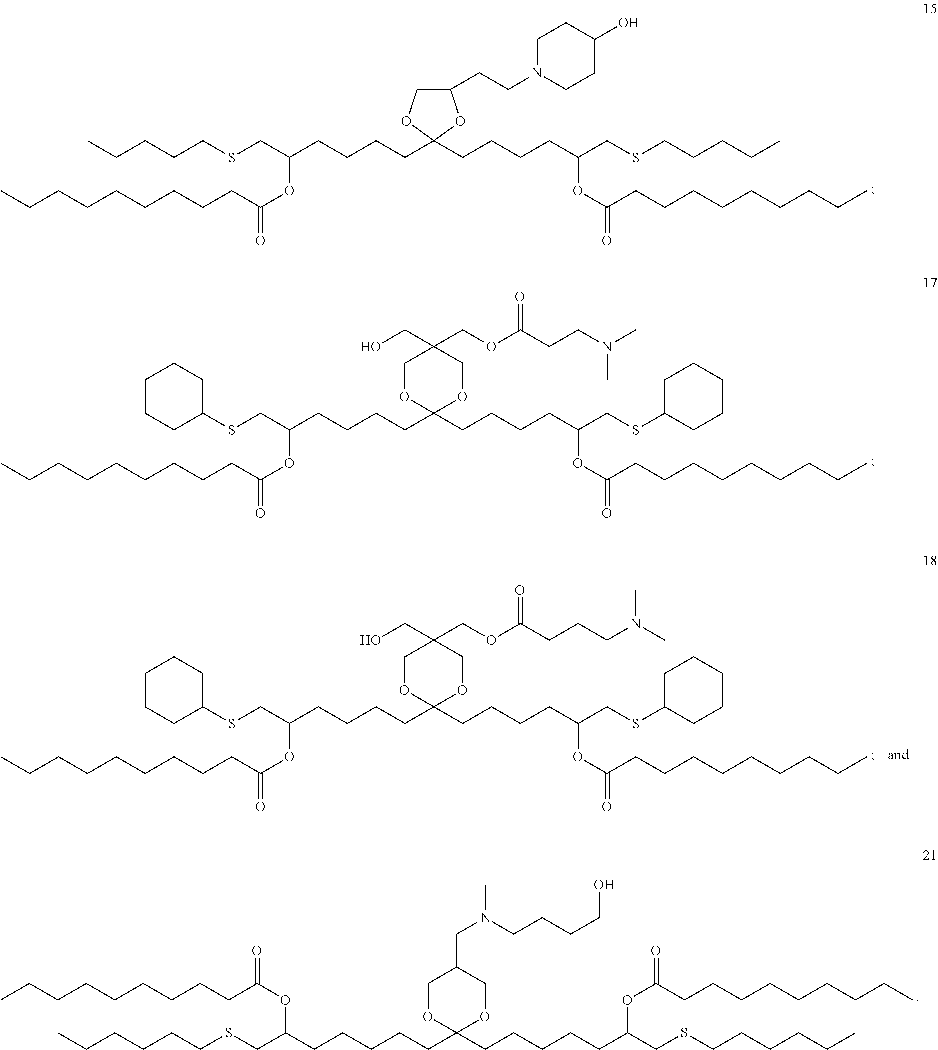

11. The lipid or the pharmaceutically acceptable salt of claim 1, wherein the lipid, when formulated in a lipid nanoparticle comprising an mRNA, results in an increase in biodistribution of the lipid nanoparticle of at least about 10% in the liver and/or one or more extrahepatic tissues relative to a lipid nanoparticle containing DLin-MC3-DMA as measured by luminescence of the mRNA in vivo in the liver and/or one or more extrahepatic tissues.

12. A lipid nanoparticle comprising the lipid of claim 1 and a nucleic acid.

13. The lipid nanoparticle of claim 12, comprising a helper lipid and optionally a hydrophilic polymer-lipid conjugate.

14. The lipid nanoparticle of claim 13, wherein the helper lipid is selected from cholesterol, a diacylglycerol, a glycerophospholipid-cholesterol conjugate and a sphingolipid.

15. A lipid nanoparticle comprising:
an ionizable lipid with two lipophilic chains directly bonded to a central nitrogen or carbon atom in which at least one of the lipophilic chains has the formula: 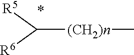
n is 4 to 8;
wherein the * represent a carbon branch point;
wherein R⁵ is a linear or branched substituted C₃-C₃₀ alkyl group;
wherein R⁶ is a linear or branched substituted C₃-C₂₀ alkyl group or is an unsubstituted, saturated C₆ alkyl;
wherein R⁵ is substituted with an ester group and R⁶ is substituted with a sulfur atom at an alpha, beta or gamma position relative to the carbon branch point;
one or more helper lipids;
optionally a hydrophilic polymer-lipid conjugate; and
a nucleic acid.

16. A method for administering a nucleic acid to a subject in need thereof, the method comprising preparing or providing the lipid nanoparticle of claim 12 comprising the nucleic acid and administering the lipid nanoparticle to the subject.

17. A method for delivering a cargo molecule to a cell, the method comprising contacting the lipid nanoparticle of claim 12 with the cell in vivo or in vitro.

18. The method of claim 17, wherein the cargo molecule is a nucleic acid.

19. (canceled)

20. (canceled)

21. (canceled)
